(19) United States
(12) Patent Application Publication
(10) Pub. No.: US 2024/0150340 A1
(43) Pub. Date: May 9, 2024

(54) CDK Inhibitors And Their Use As Pharmaceuticals

(71) Applicant: Prelude Therapeutics Incorporated, Wilmington, DE (US)

(72) Inventors: Xiaowei Wu, Wilmington, DE (US); Andrew Combs, Kennett Square, PA (US)

(21) Appl. No.: 18/482,098

(22) Filed: Oct. 6, 2023

Related U.S. Application Data
(60) Provisional application No. 63/378,542, filed on Oct. 6, 2022.

(51) Int. Cl. C07D 417/14; A61K 45/06; A61P 35/00; C07D 409/14

(52) U.S. Cl. CPC C07D 417/14; A61K 45/06; A61P 35/00; C07D 409/14

(57) ABSTRACT

The disclosure is directed to compounds of Formula I

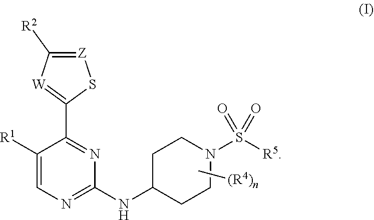

Pharmaceutical compositions comprising compounds of Formula I, as well as methods of their use and preparation, are also described.

### CROSS-REFERENCE TO RELATED APPLICATIONS

This application claims the benefit of priority to U.S. Provisional Application No. 63/378,542, filed Oct. 6, 2022, the content of which is incorporated herein by reference in its entirety.

### TECHNICAL FIELD

The disclosure is directed to CDK inhibitors and methods of their use.

### BACKGROUND

Cyclin-dependent kinases (CDKs) are a family of conserved serine/threonine kinases that play critical roles in cell cycle and gene transcription regulation (Malumbres 2014). Among the cell cycle CDK subfamily, CDK4 and CDK6 are the master regulators that control entry of cells from the first gap phase (G1) to the DNA synthesis phase (S). During this process, cyclin D protein levels increase, complex with CDK4/6 and activate their kinase activities. Activated CDK4/6 complexes phosphorylate retinoblastoma protein (RB1) and other RB1-like proteins, reduce their binding affinities and release RB1-containing transcription repressor complexes from E2F transcription factors, resulting in activation of E2F controlled cell cycle genes and progression of cell cycle (Lapenna and Giordano 2009, Asghar, Witkiewicz et al. 2015).

Additional small molecule CDK2/4/6 inhibitors are needed.

### SUMMARY OF THE INVENTION

The disclosure is directed to compounds of Formula I:

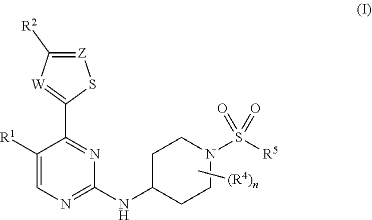

or a pharmaceutically acceptable salt or solvate or N-oxide thereof, wherein
   Z is N or CR⁶;
   W is N or CR⁶;
   n is 0, 1, 2, 3, 4, 5, 6, 7, 8, or 9;
   R¹ is selected from H, D, halogen, C₁-C₆ alkyl, C₁-C₆ alkoxide, C₁-C₆ haloalkyl, SF₅, or CN; wherein said C₁-C₆ alkyl is optionally substituted by 1-6 R groups selected from H, D, halogen, —OH, —CN, —OR^{a}, —SR^{a}, —NR^{c}R^{d};
   R² is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
   each R⁴ is independently H, D, halogen, —OH, —CN, —NO₂, —C₁-C₆alkyl, —C₁-C₆ haloalkyl, —C₁₋₆alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
   or two R⁴ together with the same atom to which they are both attached is —C(O)— or —C(S)—;
   or two R⁴ together with the carbon atom(s) to which they are both attached at same carbon or different carbons, form a carbocyclic or heterocyclic group which is optionally substituted by 1-6 R groups selected from H, D, halogen, —OH, —CN, —OR^{a}, —SR^{a}, —R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
   each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl; wherein said —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl is optionally substituted;
   each R^{b}, is independently H, D, —C₁-C₆ alkyl, —C₁-C₆ haloalkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
   each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl;
   or R^{c} and R^{d}, together with the atom to which they are both attached, form an optionally substituted monocyclic or multicyclic heterocycloalkyl, or an optionally substituted monocyclic or multicyclic heterocyclo-alkenyl group;
   R⁵ is —NR^{c}R^{d}, —NR^{a}R^{c}, C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl and heteroarylalkyl is optionally substituted by 1-6 R groups selected from H, D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b};
   or R⁴ and R⁵ together with the atoms to which they are both attached form a heterocyclic group which are optionally substituted with H, D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; and
   each R⁶ is independently H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b}.

Stereoisomers of the compounds of Formula I, as well as the pharmaceutical salts, solvates, and N-oxides thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Alkyl groups of the disclosure can be unsubstituted or substituted. In those embodiments wherein the alkyl group is substituted, the alkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “alkoxide” refers to he conjugate base of an alcohol and includes an organic group bonded to a negatively charged oxygen atom.

The term “halo” or “halogen,” refers to chloro, fluoro, bromo, or iodo.

The term “haloalkyl” refers to any alkyl radical having one or more hydrogen atoms replaced by a halogen atom.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms. Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. Cycloalkyl groups of the disclosure can be unsubstituted or substituted. In those embodiments wherein the cycloalkyl group is substituted, the cycloalkyl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “cycloalkenyl” refer to cyclic, non-aromatic hydrocarbon groups having from 3 to 10 carbon atoms (“C₃-C₁₀”), preferably from 3 to 6 carbon atoms (“C₃-C₆”) and containing at least one carbon-carbon double bond. For example, cycloalkenyl groups include, but are not limited to cyclopropenyl, cyclobutenyl, and the like.

The term “heterocycloalkyl” when used alone or as part of a substituent group refers to any three to ten membered monocyclic or bicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms. The term —C₃-C₆ heterocycloalkyl refers to a heterocycloalkyl group having between three and six carbon ring atoms. The term —C₃-C₁₀ heterocycloalkyl refers to a heterocycloalkyl group having between three and 10 ring atoms. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, and the like. Heterocycloalkyl groups of the disclosure can be unsubstituted or substituted. In those embodiments wherein the heterocycloalkyl group is substituted, the heterocycloalkyl group can be substituted with 1, 2, or 3 substituents independently selected from D, —OH, —CN, amino, halo, oxo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heterocycloalkenyl” when used alone or as part of a substituent group refers to any three to ten membered monocyclic or bicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocycloalkenyl group, the cyclic groups share two common atoms. The term —C₃-C₆ heterocycloalkenyl refers to a heterocycloalkenyl group having between three and six carbon atoms. The term —C₃-C₁₀ heterocycloalkenyl refers to a heterocycloalkenyl group having between three and ten ring atoms. The heterocycloalkenyl group may be attached at any heteroatom or carbon atom of the ring such that the result is a stable structure. Heterocycloalkenyl groups of the disclosure can be unsubstituted or substituted. In those embodiments wherein the heterocycloalkenyl group is substituted, the heterocycloalkenyl group can be substituted with 1, 2, or 3 substituents independently selected from D, —OH, —CN, amino, halo, oxo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. The term —C₅-C₁₀ heteroaryl refers to a heteroaryl group containing five to ten ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure can be unsubstituted or substituted. In those embodiments wherein the heteroaryl group is substituted, the heteroaryl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “aryl” when used alone or as part of a substituent group refers to a mono- or bicyclic-aromatic carbon ring structure. Aryl rings can include a total of 6, 7, 8, 9, or 10 ring atoms. Examples of aryl groups include but are not limited to, phenyl, napthyl, and the like. Aryl groups of the disclosure can be unsubstituted or substituted. In those embodiments wherein the aryl group is substituted, the aryl group can be substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “alkenyl” refers to C₂-C₁₂ alkyl group that contains at least one carbon-carbon double bond. In some embodiments, the alkenyl group is optionally substituted. In some embodiments, the alkenyl group is a C₂-C₆ alkenyl.

The term “alkynyl” refers to C₂-C₁₂ alkyl group that contains at least one carbon-carbon triple bond. In some embodiments, the alkenyl group is optionally substituted. In some embodiments, the alkynyl group is a C₂-C₆ alkynyl.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—. The term “—C₀alk-” refers to a bond.

Moieties of the disclosure, for example, —C₁-C₆alkyl, —C₁-C₁₀ alkyl, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl, are optionally substituted with 1, 2, or 3 substituents independently selected from D, —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

In some embodiments, the compounds of the invention, and salts thereof, are substantially isolated. By “substantially isolated” is meant that the compound is at least partially or substantially separated from the environment in which was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes mammals, and in particular, humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., enantiomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

Compounds of the invention can also include all isotopes of atoms occurring in the intermediates or final compounds. Isotopes include those atoms having the same atomic number but different mass numbers. For example, isotopes of hydrogen include tritium and deuterium.

The disclosure is directed to compounds of Formula I:

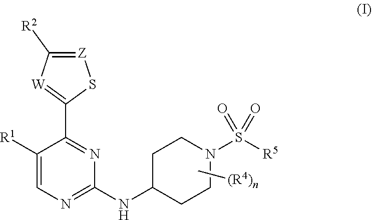

or a pharmaceutically acceptable salt or solvate or N-oxide thereof, wherein
   Z is N or CR⁶;
   W is N or CR⁶;
   n is 0, 1, 2, 3, 4, 5, 6, 7, 8, or 9;
   R¹ is selected from H, D, halogen, C₁-C₆ alkyl, C₁-C₆ alkoxide, C₁-C₆ haloalkyl, SF₅, or CN; wherein said C₁-C₆ alkyl is optionally substituted by 1-6 R groups selected from H, D, halogen, —OH, —CN, —OR^{a}, —SR^{a}, —NR^{c}R^{d};
   R² is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
   each R⁴ is independently H, D, halogen, —OH, —CN, —NO₂, —C₁-C₆alkyl, —C₁-C₆ haloalkyl, —C₁₋₆alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
   or two R⁴ together with the same atom to which they are both attached is —C(O)— or —C(S)—; or two R⁴ together with the carbon atom(s) to which they are both attached at same carbon or different carbons, form a carbocyclic or heterocyclic group which is optionally substituted by 1-6 R groups selected from H, D, halogen, —OH, —CN, —OR^{a}, —SR^{a}, —R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
   each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl; wherein said —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl is optionally substituted;
   each R^{b}, is independently H, D, —C₁-C₆ alkyl, —C₁-C₆ haloalkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
   each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl;
   or R^{c} and R^{d}, together with the atom to which they are both attached, form an optionally substituted monocyclic or multicyclic heterocycloalkyl, or an optionally substituted monocyclic or multicyclic heterocyclo-alkenyl group;
   R⁵ is —NR^{c}R^{d}, —NR^{a}R^{c}, C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl and heteroarylalkyl is optionally substituted by 1-6 R groups selected from H, D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b};
   or R⁴ and R⁵ together with the atoms to which they are both attached form a heterocyclic group which are optionally substituted with H, D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b}; and
   each R⁶ is independently H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, Z in Formula I is N or CR⁶. In some embodiments, Z in Formula I is N. In other embodiments, Z in Formula I is CR⁶.

In some embodiments, W in Formula I is N or CR⁶. In some embodiments, W in Formula I is N. In other embodiments, W in Formula I is CR⁶.

In some embodiments, both W and Z in Formula I are CR⁶. In some embodiments, W is N and Z are CR⁶. In some embodiments, W is CR⁶ and Z is N.

In some embodiments, n in Formula I is 0, 1, 2, 3, 4, 5, 6, 7, 8 or 9. In some embodiments, n is 0. In some embodiments, n is 1. In some embodiments, n is 2. In other embodiments, n is 3. In other embodiments, n is 4. In other embodiments, n is 5. In other embodiments, n is 6. In other embodiments, n is 7. In other embodiments, n is 8. In other embodiments, n is 9.

In some embodiments, R¹ in Formula I is selected from H, D, halogen, C₁-C₆ alkyl, C₁-C₆ alkoxide, C₁-C₆ haloalkyl, SF₅, or CN; wherein said C₁-C₆ alkyl is optionally substituted by 1-6 R groups selected from H, D, halogen, —OH, —CN, —OR^{a}, —SR^{a}, or —NR^{c}R^{d}.

In other embodiments, R¹ in Formula I is selected from H, D, halogen, C₁-C₆ alkyl, C₁-C₆ alkoxide, C₁-C₆ haloalkyl, SF₅, or CN; wherein said C₁-C₆ alkyl is optionally substituted by 1-6 R groups selected from D, halogen, —OH, —CN, —OR^{a}, —SR^{a}, or —NR^{c}R^{d}.

In some embodiments, R¹ is H. In some embodiments, R¹ is D. In some embodiments, R¹ is halogen. In some embodiments, R¹ is F. In other embodiments, R¹ is —C₁-C₆alkyl. In other embodiments, R¹ is C₁-C₆ alkoxide. In other embodiments, R¹ is C₁-C₆haloalkyl. In other embodiments, R¹ is CH₂F. In other embodiments, R¹ is CF₃. In other embodiments, R¹ is SF₅. In other embodiments, R¹ is CN. In other embodiments, R¹ is C₁-C₆alkyl optionally substituted by 1-6 R groups selected from H, D, halogen, —OH, —CN, —OR^{a}, —SR^{a}, and —NR^{c}R^{d}. In yet other embodiments, R¹ is C₁-C₆alkyl optionally substituted by 1-6 R groups selected from D, halogen, —OH, —CN, —OR^{a}, —SR^{a}, and —NR^{c}R^{d}.

In some embodiments, R² in Formula I is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R selected from H, D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In other embodiments, R² in Formula I is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R selected from D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R² is H. In some embodiments, R² is D. In some embodiments, R² is halogen. In some embodiments, R² is —OH. In some embodiments, R² is —CN. In some embodiments, R² is —NO₂. In some embodiments, R² is —C₁-C₆alkyl. In some embodiments, R² is —C₂-C₆alkenyl. In some embodiments, R² is —C₂-C₆alkynyl. In some embodiments, R² is aryl. In other embodiments, R² is independently heteroaryl. In other embodiments, R² is cycloalkyl. In other embodiments, R² is cycloalkenyl. In other embodiments, R² is heterocycloalkyl. In other embodiments, R² is heterocycloalkenyl. In other embodiments, R² is cycloalkylalkyl. In other embodiments, R² is heterocycloalkylalkyl. In yet other embodiments, R² is arylalkyl. In yet other embodiments, R² is heteroarylalkyl. In yet other embodiments, R² is OR^{a}. In yet other embodiments, R² is SR^{a}. In yet other embodiments, R² is NR^{c}R^{d}. In yet other embodiments, R² is NR^{a}R^{c}. In yet other embodiments, R² is R^{b}. In yet other embodiments, R² is —C(O)R^{b}. In yet other embodiments, R² is —OC(O)R^{b}. In yet other embodiments, R² is —C(O)OR^{b}. In yet other embodiments, R² is —C(O)NR^{c}R^{d}. In yet other embodiments, R² is —C(═NR^{b})NR^{c}R^{d}. In yet other embodiments, R² is —C(═NR^{b})R^{b}. In yet other embodiments, R² is —S(O)R^{b}. In yet other embodiments, R² is —S(O)₂NR^{c}R^{d}. In yet other embodiments, R² is —S(O)(═NR^{b})R^{b}. In yet other embodiments, R² is —SF₅. In some embodiments, R² is —P(O)R^{b}R^{b}. In yet other embodiments, R² is —P(O)(OR^{b})(OR^{b}). In yet other embodiments, R² is —B(OR^{c})(OR^{d}). In yet other embodiments, R² is —S(O)₂R^{b}.

In some embodiments, the C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocyclo-alkylalkyl, arylalkyl, or heteroarylalkyl groups of R² are substituted by 1-6 R groups selected from H, D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}. In other embodiments, the C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocyclo-alkylalkyl, arylalkyl, or heteroarylalkyl groups of R² are substituted by 1-6 R groups selected from D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, each R⁴ in Formula I is selected from H, D, halogen, —OH, —CN, —NO₂, —C₁-C₆alkyl, —C₁-C₆ haloalkyl, —C₁₋₆alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or two R⁴ together with the same atom to which they are both attached is —C(O)— or —C(S)—;
or two R⁴ together with the carbon atom(s) to which they are both attached at same carbon or different carbons, form a carbocyclic or heterocyclic group which is optionally substituted by 1-6 R groups selected from H, D, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}. In other embodiments, two R⁴ together with the carbon atom(s) to which they are both attached at same carbon or different carbons, form a carbocyclic or heterocyclic group which is optionally substituted by 1-6 R groups selected from D, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In other embodiments, each R⁴ in Formula I is selected from D, halogen, —OH, —CN, —NO₂, —C₁-C₆alkyl, —C₁-C₆ haloalkyl, —C₁₋₆alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁴ is H. In some embodiments, R⁴ is D. In some embodiments, R⁴ is halogen. In some embodiments, R⁴ is fluorine. In some embodiments, R⁴ is —OH. In some embodiments, R⁴ is —CN. In some embodiments, R⁴ is —NO₂. In some embodiments, R⁴ is —C₁-C₆alkyl. In some embodiments, R⁴ is methyl. In some embodiments, R⁴ is —C₁-C₆ haloalkyl. In other embodiments, R⁴ is C₁₋₆alkoxide. In other embodiments, R⁴ is —C₂-C₆alkenyl. In other embodiments, R⁴ is —C₂-C₆alkynyl. In other embodiments, R⁴ is aryl. In other embodiments, R⁴ is independently heteroaryl. In other embodiments, R⁴ is cycloalkyl. In other embodiments, R⁴ is cycloalkenyl. In other embodiments, R⁴ is heterocycloalkyl. In other embodiments, R⁴ is heterocycloalkenyl. In yet other embodiments, R⁴ is OR^{a}. In yet other embodiments, R⁴ is SR^{a}. In yet other embodiments, R⁴ is NR^{c}R^{d}. In yet other embodiments, R⁴ is NR^{a}R^{c}. In yet other embodiments, R⁴ is —C(O)R^{b}. In yet other embodiments, R⁴ is —OC(O)R^{b}. In yet other embodiments, R⁴ is —C(O)OR^{b}. In yet other embodiments, R⁴ is —C(O)NR^{c}R^{d}. In yet other embodiments, R⁴ is —S(O)R^{b}. In yet other embodiments, R⁴ is —S(O)₂NR^{c}R^{d}. In yet other embodiments, R⁴ is —S(O)(═NR^{b})R^{b}. In yet other embodiments, R⁴ is —SF₅. In some embodiments, R⁴ is —P(O)R^{b}R^{b}. In yet other embodiments, R⁴ is —P(O)(OR^{b})(OR^{b}). In yet other embodiments, R⁴ is —B(OR)(OR^{d}). In yet other embodiments, R⁴ is —S(O)₂R^{b}.

In some embodiments, two R⁴ together with the same atom to which they are both attached is —C(O)— or —C(S)—.

In some embodiments, two R⁴ together with the carbon atom(s) to which they are both attached at same carbon or different carbons, form a carbocyclic or heterocyclic group which is optionally substituted by 1-6 R groups selected from H, D, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b}. In other embodiments, two R⁴ together with the carbon atom(s) to which they are both attached at same carbon or different carbons, form a carbocyclic or heterocyclic group which is optionally substituted by 1-6 R groups selected from D, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR)(OR^{d}) or —S(O)₂R^{b}.

In some embodiments, each R^{a} in Formula I is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl.

In some embodiments, R^{a} is independently H. In some embodiments, R^{a} is independently D. In some embodiments, R^{a} is independently —C(O)R^{b}. In some embodiments, R^{a} is independently —C(O)OR^{c}. In some embodiments, R^{a} is independently —C(O)NR^{c}R^{d}. In some embodiments, R^{a} is independently —C(═NR^{b})NR^{b}R^{c}. In some embodiments, R^{a} is independently C(═NOR^{b})NR^{b}R^{c}. In some embodiments, R^{a} is independently —C(═NCN)NR^{b}R^{c}.

In other embodiments, R^{a} is independently P(OR^{c})₂, —P(O)OR^{c}OR^{b}, —S(O)₂R^{b}, —S(O)₂NR^{c}R^{d}, SiR^{b}₃, and the like. In yet other embodiments, R^{a} is independently —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl, and the like.

In some embodiments, each R^{b} in Formula I is independently H, D, —C₁-C₆ alkyl, —C₁-C₆ haloalkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl. In some embodiments, R^{b} is independently H. In some embodiments, R^{b} is independently D. In some embodiments, R^{b} is independently —C₁-C₆ alkyl. In some embodiments, R^{b} is independently —C₁-C₆ haloalkyl. In some embodiments, R^{b} is independently —C₂-C₆ alkenyl. In some embodiments, R^{b} is independently —C₂-C₆ alkynyl. In other embodiments, R^{b} is independently C₀-C₁alk-aryl. In other embodiments, R^{b} is independently cycloalkyl. In other embodiments, R^{b} is independently cycloalkenyl. In other embodiments, R^{b} is independently C₀-C₁alk-heteroaryl. In other embodiments, R^{b} is independently heterocycloalkyl. In other embodiments, R^{b} is independently heterocycloalkenyl.

In some embodiments, each R^{c} in Formula I is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl.

In some embodiments, R^{c} is independently H. In some embodiments, R^{c} is independently D. In some embodiments, R^{c} is independently —C₁-C₁₀ alkyl. In some embodiments, R^{c} is independently —C₂-C₆ alkenyl. In some embodiments, R^{c} is independently —C₂-C₆ alkynyl. In other embodiments, R^{c} is independently —OC₁-C₆alkyl. In other embodiments, R^{c} is independently —O— cycloalkyl. In other embodiments, R^{c} is independently aryl. In other embodiments, R^{c} is independently C₁alk-aryl. In other embodiments, R^{c} is independently heteroaryl. In other embodiments, R^{c} is independently cycloalkyl. In other embodiments, R^{c} is independently cycloalkenyl. In other embodiments, R^{c} is independently C₁alk-heteroaryl. In other embodiments, R^{c} is independently heterocycloalkyl. In other embodiments, R^{c} is independently heterocycloalkenyl.

In some embodiments, each R^{d} in Formula I is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl.

In some embodiments, R^{d} is independently H. In some embodiments, R^{d} is independently D. In some embodiments, R^{d} is independently —C₁-C₁₀ alkyl. In some embodiments, R^{d} is independently —C₂-C₆ alkenyl. In some embodiments, R^{d} is independently —C₂-C₆ alkynyl. In other embodiments, R^{d} is independently —OC₁-C₆alkyl. In other embodiments, R^{d} is independently —O— cycloalkyl. In other embodiments, R^{d} is independently aryl. In other embodiments, R^{d} is independently C₁alk-aryl. In other embodiments, R^{d} is independently heteroaryl. In other embodiments, R^{d} is independently cycloalkyl. In other embodiments, R^{d} is independently cycloalkenyl. In other embodiments, R^{d} is independently C₁alk-heteroaryl. In other embodiments, R^{d} is independently heterocycloalkyl. In other embodiments, R^{d} is independently heterocycloalkenyl.

In some embodiments, R^{c} and R^{d}, together with the atom to which they are both attached, form an optionally substituted monocyclic or multicyclic heterocycloalkyl, or an optionally substituted monocyclic or multicyclic heterocyclo-alkenyl group.

In some embodiments, R⁵ in Formula I is selected from —NR^{c}R^{d}, —NR^{a}R^{c}, C₁₋₆alkyl, C₃₋₇ cycloalkyl, C₄₋₇heterocycloalkyl, C₃₋₇cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl and heteroarylalkyl is optionally substituted by 1-6 R groups selected from H, D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁵ in Formula I is selected from —NR^{c}R^{d}, —NR^{a}R^{c}, C₁₋₆alkyl, C₃₋₇ cycloalkyl, C₄₋₇heterocycloalkyl, C₃₋₇cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄₋₇heterocycloalkylalkyl, aryl, heteroaryl, arylalkyl and heteroarylalkyl is optionally substituted by 1-6 R groups selected from D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁵ is NR^{c}R^{d}. In some embodiments, R⁵ is NR^{a}R^{c}. In some embodiments, R⁵ is —C₁-C₆alkyl. In some embodiments, R⁵ is methyl. In some embodiments, R⁵ is C₃₋₇cycloalkyl. In other embodiments, R⁵ is C₄₋₇heterocycloalkyl. In other embodiments, R⁵ is C₃₋₇ cycloalkylalkyl. In other embodiments, R⁵ is C₄₋₇heterocycloalkylalkyl. In other embodiments, R⁵ is aryl. In yet other embodiments, R⁵ is heteroaryl. In yet other embodiments, R⁵ is arylalkyl. In yet other embodiments, R⁵ is heteroarylalkyl. In yet other embodiments, R⁵ is haloalkyl.

In some embodiments, the C₁₋₆alkyl, C₃₋₇cycloalkyl, C₄₋₇heterocycloalkyl, C₃₋₇ cycloalkylalkyl, C₄₋₇heterocycloalkylalkyl, aryl, heteroaryl, arylalkyl or heteroarylalkyl groups of R⁵ are substituted by 1-6 R groups selected from H, D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}. In other embodiments, the C₁₋₆alkyl, C₃₋₇cycloalkyl, C₄₋₇ heterocycloalkyl, C₃₋₇cycloalkylalkyl, C₄₋₇heterocycloalkylalkyl, aryl, heteroaryl, arylalkyl or heteroarylalkyl groups of R⁵ are substituted by 1-6 R groups selected from D, Oxo, halogen, alkyl, haloalkyl, cycloalkyl, heterocycloalkyl, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁵ is a phenyl, a furan, a thiophene, a pyridine, an imidazole, a pyrazole, a thiazole or a pyrazolo-pyridazine; wherein the phenyl, furan, thiophene, pyridine, imidazole, pyrazole, thiazole or pyrazolo-pyridazine is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}. In other embodiments, R⁵ is a phenyl, a furan, a thiophene, a pyridine, an imidazole, a pyrazole, a thiazole or a pyrazolo-pyridazine; wherein the phenyl, furan, thiophene, pyridine, imidazole, pyrazole, thiazole or pyrazolo-pyridazine is optionally substituted by 1-6 R groups selected from D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}. In some embodiments, R⁵ is phenyl. In some embodiments, R⁵ is furan. In some embodiments, R⁵ is thiophene. In other embodiments, R⁵ is pyridine. In other embodiments, R⁵ is imidazole. In other embodiments, R⁵ is pyrazole. In yet other embodiments, R⁵ is thiazole. In yet other embodiments, R⁵ is pyrazolo-pyridazine.

In some embodiments, the R⁴ and R⁵ groups together with the atoms to which they are both attached form a heterocyclic group which are optionally substituted with D, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁶ in Formula I is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R selected from H, D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁶ in Formula I is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R selected from D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R⁶ is H. In some embodiments, R⁶ is D. In some embodiments, R⁶ is halogen. In some embodiments, R⁶ is —OH. In some embodiments, R⁶ is —CN. In some embodiments, R⁶ is —NO₂. In some embodiments, R⁶ is —C₁-C₆alkyl. In some embodiments, R⁶ is —C₂-C₆alkenyl. In some embodiments, R⁶ is —C₂-C₆alkynyl. In some embodiments, R⁶ is aryl. In other embodiments, R⁶ is independently heteroaryl. In other embodiments, R⁶ is cycloalkyl. In other embodiments, R⁶ is cycloalkenyl. In other embodiments, R⁶ is heterocycloalkyl. In other embodiments, R⁶ is heterocycloalkenyl. In other embodiments, R⁶ is cycloalkylalkyl. In other embodiments, R⁶ is heterocycloalkylalkyl. In yet other embodiments, R⁶ is arylalkyl. In yet other embodiments, R⁶ is heteroarylalkyl. In yet other embodiments, R⁶ is OR^{a}. In yet other embodiments, R⁶ is SR^{a}. In yet other embodiments, R⁶ is NR^{c}R^{d}. In yet other embodiments, R⁶ is NR^{a}R^{c}. In yet other embodiments, R⁶ is R^{b}. In yet other embodiments, R⁶ is —C(O)R^{b}. In yet other embodiments, R⁶ is —OC(O)R^{b}. In yet other embodiments, R⁶ is —C(O)OR^{b}. In yet other embodiments, R⁶ is —C(O)NR^{c}R^{d}. In yet other embodiments, R⁶ is —C(═NR^{b})NR^{c}R^{d}. In yet other embodiments, R⁶ is —C(═NR^{b})R^{b}. In yet other embodiments, R⁶ is —S(O)R^{b}. In yet other embodiments, R⁶ is —S(O)₂NR^{c}R^{d}. In yet other embodiments, R⁶ is —S(O)(═NR^{b})R^{b}. In yet other embodiments, R⁶ is —SF₅. In some embodiments, R⁶ is —P(O)R^{b}R^{b}. In yet other embodiments, R⁶ is —P(O)(OR^{b})(OR^{b}). In yet other embodiments, R⁶ is —B(OR^{c})(OR^{d}). In yet other embodiments, R⁶ is —S(O)₂R^{b}.

In some embodiments, the C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl groups of R⁶ are substituted by 1-6 R groups selected from H, D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}. In other embodiments, the C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl groups of R⁶ are substituted by 1-6 R groups selected from D, oxo, halogen, —OH, —CN, —OR^{a}, —SR^{a}, R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, the compounds of Formula (I) are the pharmaceutically acceptable salts. In some embodiments, the compounds of Formula (I) are solvates. In some embodiments, the compounds of Formula (I) are N-oxides of the compounds of Formula (I).

In some embodiments, the compounds of Formula (I) are represented by compounds of Formula II

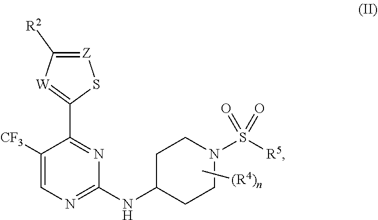

or a pharmaceutically acceptable salt or solvate or N-oxide thereof; wherein each R², (R⁴)ₙ, R⁵, W and Z are defined with respect to Formula (I).

In some embodiments, the compounds of Formula (I) are represented by compounds of Formula III

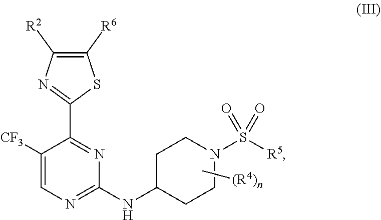

or a pharmaceutically acceptable salt or solvate or N-oxide thereof; wherein each R², (R⁴)ₙ, R⁵ and R⁶ are defined with respect to Formula (I).

In some embodiments, the compounds of Formula (I) are represented by compounds of Formula IV:

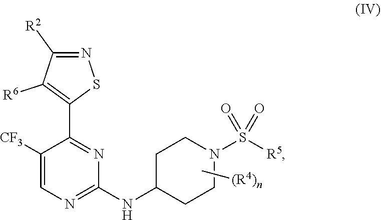

or a pharmaceutically acceptable salt or solvate or N-oxide thereof; wherein each R², (R⁴)ₙ, R⁵ and R⁶ are defined with respect to Formula (I).

In some embodiments, the compounds of Formula (I) are represented by compounds of Formula V:

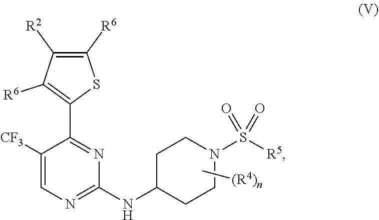

or a pharmaceutically acceptable salt or solvate or N-oxide thereof; wherein each R², (R⁴)ₙ, R⁵ and R⁶ are defined with respect to Formula (I).

In some embodiments, the compounds of Formula (I) are represented by compounds of Formula VI:

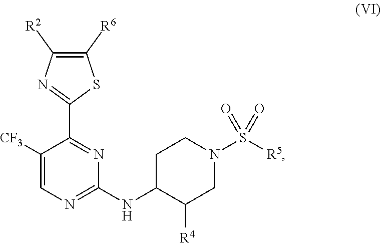

or a pharmaceutically acceptable salt or solvate or N-oxide thereof; wherein each R², R⁴, R⁵ and R⁶ are defined with respect to Formula (I).

In some embodiments, the compounds of Formula (I) are represented by compounds of Formula VII:

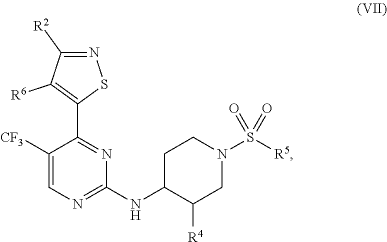

or a pharmaceutically acceptable salt or solvate or N-oxide thereof; wherein each R², R⁴, R⁵ and R⁶ are defined with respect to Formula (I).

In some embodiments, the compounds of Formula (I) are represented by compounds of Formula VIII:

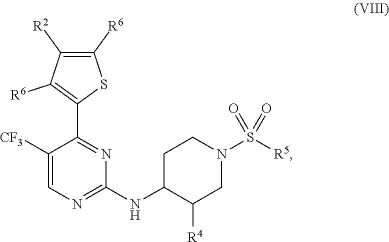

or a pharmaceutically acceptable salt or solvate or N-oxide thereof; wherein each R², R⁴, R⁵ and R⁶ are defined with respect to Formula (I).

In yet further embodiments, the compounds of Formula (I) are:
2-Methyl-5-[2-[[1-(1-methylimidazol-4-yl)sulfonylpiperidin-4-yl]amino]-5-(trifluoro-methyl)pyrimidin-4-yl]thiophene-3-carbonitrile;
N-(1-((1-methyl-1H-pyrazol-4-yl)sulfonyl)piperidin-4-yl)-4-(5-methylthiazol-2-yl)-5-(trifluoromethyl)pyrimidin-2-amine;
2-Methyl-1-(2-(2-((1-((1-methyl-1H-imidazol-4-yl)sulfonyl)piperidin-4-yl)amino)-5-(trifluoromethyl)pyrimidin-4-yl)thiazol-5-yl)propan-2-ol;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carbonitrile;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-methylthiophene-3-carbonitrile;
5-(2-(((3R,4R)-3-fluoro-1-(methylsulfonyl)piperidin-4-yl)amino)-5-(trifluoromethyl)-pyrimidin-4-yl)thiophene-3-carboxamide;
5-(2-(((3R,4S)-3-fluoro-1-(methylsulfonyl)piperidin-4-yl)amino)-5-(trifluoromethyl)-pyrimidin-4-yl)thiophene-3-carboxamide;
5-(2-(((3R,4R)-3-fluoro-1-(methylsulfonyl)piperidin-4-yl)amino)-5-(trifluoromethyl)-pyrimidin-4-yl)-2-methylthiophene-3-carboxamide;
5-(2-(((3R,4S)-3-fluoro-1-(methylsulfonyl)piperidin-4-yl)amino)-5-(trifluoromethyl)-pyrimidin-4-yl)-2-methylthiophene-3-carboxamide;
5-(2-(((3R,4S)-3-methyl-1-(methylsulfonyl)piperidin-4-yl)amino)-5-(trifluoromethyl)-pyrimidin-4-yl)thiophene-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiophene-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiophene-3-carboxamide;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-methylthiophene-3-carboxamide;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carboxamide;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-methylthiophene-3-carboxamide;
5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carboxamide;
5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-1-((1H-imidazol-4-yl)sulfonyl)-3-methylpiperidin-4-yl)amino)-5-(trifluoromethyl)pyrimidin-4-yl)thiophene-3-carboxamide;
5-(2-(((3R,4S)-1-(imidazo[1,2-b]pyridazin-3-ylsulfonyl)-3-methylpiperidin-4-yl)amino)-5-(trifluoromethyl)pyrimidin-4-yl)thiophene-3-carboxamide;
2-methyl-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carboxamide;
2-methyl-5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-1-((1H-imidazol-4-yl)sulfonyl)-3-methylpiperidin-4-yl)amino)-5-(trifluoro-methyl)pyrimidin-4-yl)-2-methylthiophene-3-carboxamide;
N-((3R,4S)-3-fluoro-1-((1-methyl-1H-pyrazol-4-yl)sulfonyl)piperidin-4-yl)-4-(5-methylthiazol-2-yl)-5-(trifluoromethyl)pyrimidin-2-amine;
N-((3R,4R)-3-fluoro-1-((1-methyl-1H-pyrazol-4-yl)sulfonyl)piperidin-4-yl)-4-(5-methylthiazol-2-yl)-5-(trifluoromethyl)pyrimidin-2-amine;
N-((3R,4S)-3-fluoro-1-((1-methyl-1H-imidazol-4-yl)sulfonyl)piperidin-4-yl)-4-(5-methylthiazol-2-yl)-5-(trifluoromethyl)pyrimidin-2-amine;
N-((3R,4R)-3-fluoro-1-((1-methyl-1H-imidazol-4-yl)sulfonyl)piperidin-4-yl)-4-(5-methylthiazol-2-yl)-5-(trifluoromethyl)pyrimidin-2-amine;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carbonitrile;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-methylthiophene-3-carbonitrile;
N-(1-((1-methyl-1H-imidazol-4-yl)sulfonyl)piperidin-4-yl)-4-(5-methylthiazol-2-yl)-5-(trifluoromethyl)pyrimidin-2-amine;
2-(1-hydroxyethyl)-5-(2-((1-((1-methyl-1H-imidazol-4-yl)sulfonyl)piperidin-4-yl)amino)-5-(trifluoromethyl)pyrimidin-4-yl)thiophene-3-carbonitrile;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-(1-hydroxyethyl)thiophene-3-carbonitrile;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-(1-hydroxyethyl)thiophene-3-carbonitrile;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-(1-hydroxyethyl)thiophene-3-carbonitrile;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-(1-hydroxyethyl)thiophene-3-carbonitrile;
2-(2-hydroxy-2-methylpropyl)-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carbonitrile;
2-(2-hydroxy-2-methylpropyl)-5-(2-((1-((1-methyl-1H-imidazol-4-yl)sulfonyl)piperidin-4-yl)amino)-5-(trifluoromethyl)pyrimidin-4-yl)thiophene-3-carbonitrile;
2-(2-hydroxy-2-methylpropyl)-5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carbonitrile;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carbonitrile;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carbonitrile;
2-(2-hydroxy-2-methylpropyl)-5-(2-(((3R,4S)-3-methyl-1-(methylsulfonyl)piperidin-4-yl)amino)-5-(trifluoromethyl)pyrimidin-4-yl)thiophene-3-carboxamide;
2-(2-hydroxy-2-methylpropyl)-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carboxamide;
2-(2-hydroxy-2-methylpropyl)-5-(2-((1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-((1-((1-methyl-1H-imidazol-4-yl)sulfonyl)piperidin-4-yl)amino)-5-(trifluoro-methyl)pyrimidin-4-yl)isothiazole-3-carboxamide;
2-(2-hydroxy-2-methylpropyl)-5-(2-((1-((1-methyl-1H-pyrazol-4-yl)sulfonyl)piperidin-4-yl)amino)-5-(trifluoromethyl)pyrimidin-4-yl)thiophene-3-carboxamide;
2-methyl-5-(2-(((3R,4S)-3-methyl-1-(methylsulfonyl)piperidin-4-yl)amino)-5-(trifluoro-methyl)pyrimidin-4-yl)thiophene-3-carboxamide;
2-methyl-5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carbonitrile;
2-methyl-5-(2-(((3R,4S)-3-methyl-1-(methylsulfonyl)piperidin-4-yl)amino)-5-(trifluoro-methyl)pyrimidin-4-yl)thiophene-3-carbonitrile;
5-(2-((1-((1H-imidazol-4-yl)sulfonyl)piperidin-4-yl)amino)-5-(trifluoromethyl)pyrimidin-4-yl)-2-methylthiophene-3-carbonitrile;
1-(2-(2-((1-((1H-pyrazol-4-yl)sulfonyl)piperidin-4-yl)amino)-5-(trifluoromethyl)pyrimidin-4-yl)thiazol-5-yl)ethan-1-ol;
1-(2-(2-((1-((1H-imidazol-4-yl)sulfonyl)piperidin-4-yl)amino)-5-(trifluoromethyl)pyrimidin-4-yl)thiazol-5-yl)ethan-1-ol;
N-(1-((1-methyl-1H-pyrazol-4-yl)sulfonyl)piperidin-4-yl)-4-(thiazol-2-yl)-5-(trifluoro-methyl)pyrimidin-2-amine;
N-(1-((1-methyl-1H-imidazol-4-yl)sulfonyl)piperidin-4-yl)-4-(thiazol-2-yl)-5-(trifluoro-methyl)pyrimidin-2-amine;
2,2,2-trifluoro-1-(2-(2-((1-((1-methyl-1H-imidazol-4-yl)sulfonyl)piperidin-4-yl)amino)-5-(trifluoromethyl)pyrimidin-4-yl)thiazol-5-yl)ethan-1-ol;
2,2,2-trifluoro-1-(2-(2-((1-((1-methyl-1H-pyrazol-4-yl)sulfonyl)piperidin-4-yl)amino)-5-(trifluoromethyl)pyrimidin-4-yl)thiazol-5-yl)ethan-1-ol;
2-methyl-1-(2-(2-((1-((1-methyl-1H-pyrazol-4-yl)sulfonyl)piperidin-4-yl)amino)-5-(trifluoromethyl)pyrimidin-4-yl)thiazol-5-yl)propan-2-ol;
1-(2-(2-((1-((1-methyl-1H-imidazol-4-yl)sulfonyl)piperidin-4-yl)amino)-5-(trifluoromethyl)-pyrimidin-4-yl)thiazol-5-yl)ethan-1-ol;
N-((3R,4S)-3-methyl-1-((1-methyl-1H-imidazol-4-yl)sulfonyl)piperidin-4-yl)-4-(5-methylthiazol-2-yl)-5-(trifluoromethyl)pyrimidin-2-amine;
N-(1-(methylsulfonyl)piperidin-4-yl)-4-(5-methylthiazol-2-yl)-5-(trifluoromethyl)pyrimidin-2-amine;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)isothiazole-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)isothiazole-3-carboxamide;
5-(2-(((3R,4S)-1-((1H-imidazol-4-yl)sulfonyl)-3-methylpiperidin-4-yl)amino)-5-(trifluoro-methyl)pyrimidin-4-yl)-2-(1-hydroxyethyl)thiophene-3-carbonitrile;
2-methyl-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carbonitrile;
5-(2-((1-((1-Methyl-1H-pyrazol-4-yl)sulfonyl)piperidin-4-yl)amino)-5-(trifluoro-methyl)pyrimidin-4-yl)thiophene-3-carbonitrile;
2-methyl-5-(2-((1-(methylsulfonyl)piperidin-4-yl)amino)-5-(trifluoromethyl)pyrimidin-4-yl)thiophene-3-carboxamide;
2-methyl-5-(2-((1-((1-methyl-1H-pyrazol-4-yl)sulfonyl)piperidin-4-yl)amino)-5-(trifluoro-methyl)pyrimidin-4-yl)thiophene-3-carboxamide;
2-methyl-5-(2-((1-((1-methyl-1H-pyrazol-4-yl)sulfonyl)piperidin-4-yl)amino)-5-(trifluoro-methyl)pyrimidin-4-yl)thiophene-3-carbonitrile;
5-(2-((1-(methylsulfonyl)piperidin-4-yl)amino)-5-(trifluoromethyl)pyrimidin-4-yl)thiophene-3-carboxamide;
5-(2-((1-((1-methyl-1H-pyrazol-4-yl)sulfonyl)piperidin-4-yl)amino)-5-(trifluoromethyl)-pyrimidin-4-yl)thiophene-3-carboxamide;
4-(4-methoxy-5-methylthiophen-2-yl)-N-(1-((1-methyl-1H-imidazol-4-yl)sulfonyl)piperidin-4-yl)-5-(trifluoromethyl)pyrimidin-2-amine;
4-(4-methoxy-5-methylthiophen-2-yl)-N-(1-(methylsulfonyl)piperidin-4-yl)-5-(trifluoro-methyl)pyrimidin-2-amine;
N-(1-((1-methyl-1H-pyrazol-4-yl)sulfonyl)piperidin-4-yl)-4-(3-methylisothiazol-5-yl)-5-(trifluoromethyl)pyrimidin-2-amine;
4-(3-methylisothiazol-5-yl)-N-(1-(methylsulfonyl)piperidin-4-yl)-5-(trifluoromethyl)-pyrimidin-2-amine;
4-(4-(difluoromethyl)-5-methylthiophen-2-yl)-N-(1-((1-methyl-1H-pyrazol-4-yl)sulfonyl)-piperidin-4-yl)-5-(trifluoromethyl)pyrimidin-2-amine;
4-(4-(difluoromethyl)-5-methylthiophen-2-yl)-N-(1-(methylsulfonyl)piperidin-4-yl)-5-(trifluoromethyl)pyrimidin-2-amine;
1-(2-methyl-5-(2-((1-((1-methyl-1H-pyrazol-4-yl)sulfonyl)piperidin-4-yl)amino)-5-(trifluoromethyl)pyrimidin-4-yl)thiophen-3-yl)ethan-1-one;
4-(4-(methoxymethyl)-5-methylthiophen-2-yl)-N-(1-((1-methyl-1H-pyrazol-4-yl)sulfonyl)piperidin-4-yl)-5-(trifluoromethyl)pyrimidin-2-amine;
2-methyl-5-(2-((1-(methylsulfonyl)piperidin-4-yl)amino)-5-(trifluoromethyl)pyrimidin-4-yl)thiophene-3-carbonitrile;
5-(2-((1-(methylsulfonyl)piperidin-4-yl)amino)-5-(trifluoromethyl)pyrimidin-4-yl)isothiazole-3-carboxamide;
5-(2-(((3R,4S)-1-((1H-imidazol-4-yl)sulfonyl)-3-methylpiperidin-4-yl)amino)-5-(trifluoro-methyl)pyrimidin-4-yl)-2-methylthiophene-3-carbonitrile;
5-(2-(((3R,4S)-1-((1-(Cyanomethyl)-1H-pyrazol-4-yl)sulfonyl)-3-methylpiperidin-4-yl)amino)-5-(trifluoromethyl)pyrimidin-4-yl)thiophene-3-carboxamide;
5-(2-((1-((1-(cyanomethyl)-1H-pyrazol-4-yl)sulfonyl)piperidin-4-yl)amino)-5-(trifluoro-methyl)pyrimidin-4-yl)-2-methylthiophene-3-carbonitrile;
1-(2-(2-((1-((1-Methyl-1H-pyrazol-4-yl)sulfonyl)piperidin-4-yl)amino)-5-(trifluoromethyl)-pyrimidin-4-yl)thiazol-5-yl)ethan-1-ol;
2-(1-Hydroxyethyl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile;
5-(2-((1-((1-methyl-1H-pyrazol-4-yl)sulfonyl)piperidin-4-yl)amino)-5-(trifluoromethyl)-pyrimidin-4-yl)isothiazole-3-carbonitrile;
2-(2-((1-((1-Methyl-1H-imidazol-4-yl)sulfonyl)piperidin-4-yl)amino)-5-(trifluoromethyl)-pyrimidin-4-yl)thiazole-4-carboxamide;
5-(2-((1-((2-Aminothiazol-5-yl)sulfonyl)piperidin-4-yl)amino)-5-(trifluoromethyl)pyrimidin-4-yl)-2-methylthiophene-3-carbonitrile;
5-(2-(((3R,4S)-1-((2-aminothiazol-5-yl)sulfonyl)-3-methylpiperidin-4-yl)amino)-5-(trifluoro-methyl)pyrimidin-4-yl)-2-methylthiophene-3-carbonitrile;
   or a pharmaceutically acceptable salt thereof.

In yet further embodiments, the compounds of Formula (I) are:
2-(2-(2-((1-(methylsulfonyl)piperidin-4-yl)amino)-5-(trifluoromethyl)pyrimidin-4-yl)thiazol-5-yl)propan-2-ol;
2-(2-(2-(((3R,4S)-3-fluoro-1-(methylsulfonyl)piperidin-4-yl)amino)-5-(trifluoromethyl) pyrimidin-4-yl)thiazol-5-yl)propan-2-ol;
2-(2-(2-(((3R,4R)-3-fluoro-1-(methylsulfonyl)piperidin-4-yl)amino)-5-98(trifluoromethyl) pyrimidin-4-yl)thiazol-5-yl)propan-2-ol;
2-(2-(2-((1-((1-methyl-1H-imidazol-4-yl)sulfonyl)piperidin-4-yl)amino)-5-(trifluoromethyl) pyrimidin-4-yl)thiazol-5-yl)propan-2-ol;
2-(2-(2-((1-((1-methyl-1H-pyrazol-4-yl)sulfonyl)piperidin-4-yl)amino)-5-(trifluoromethyl) pyrimidin-4-yl)thiazol-5-yl)propan-2-ol;
2-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
2-(2-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiazol-5-yl)propan-2-ol;
1,1-difluoro-2-(2-(2-((1-(methylsulfonyl)piperidin-4-yl)amino)-5-(trifluoromethyl) pyrimidin-4-yl)thiazol-5-yl)propan-2-ol;
1,1,1-trifluoro-2-(2-(2-((1-(methylsulfonyl)piperidin-4-yl)amino)-5-(trifluoromethyl) pyrimidin-4-yl)thiazol-5-yl)propan-2-ol;
1,1-difluoro-2-(2-(2-(((3R,4S)-3-methyl-1-((1-methyl-1H-imidazol-4-yl)sulfonyl)piperidin-4-yl)amino)-5-(trifluoromethyl)pyrimidin-4-yl)thiazol-5-yl)propan-2-ol;
1,1,1-trifluoro-2-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
1-(2-(2-((1-(methylsulfonyl)piperidin-4-yl)amino)-5-(trifluoromethyl)pyrimidin-4-yl)thiazol-5-yl)cyclobutan-1-ol;
2-methyl-1-(2-(2-((1-(methylsulfonyl)piperidin-4-yl)amino)-5-(trifluoromethyl)pyrimidin-4-yl)thiazol-5-yl)propan-2-ol;
1-(2-(2-(((3R,4S)-3-fluoro-1-(methylsulfonyl)piperidin-4-yl)amino)-5-(trifluoromethyl) pyrimidin-4-yl)thiazol-5-yl)-2-methylpropan-2-ol;
1-(2-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiazol-5-yl)-2-methylpropan-2-ol;
1-(2-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiazol-5-yl)-2-methylpropan-2-ol;
2-methyl-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
1-(2-(2-((1-(methylsulfonyl)piperidin-4-yl)amino)-5-(trifluoromethyl)pyrimidin-4-yl)thiazol-5-yl)ethan-1-ol;
2,2,2-trifluoro-1-(2-(2-((1-(methylsulfonyl)piperidin-4-yl)amino)-5-(trifluoromethyl) pyrimidin-4-yl)thiazol-5-yl)ethan-1-ol;
1-(2-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiazol-5-yl)ethan-1-ol;
1-(2-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiazol-5-yl)ethan-1-ol;
5-(2-(((3R,4R)-3-fluoro-1-(methylsulfonyl)piperidin-4-yl)amino)-5-(trifluoromethyl) pyrimidin-4-yl)-2-(2-hydroxypropan-2-yl)thiophene-3-carbonitrile;
2-(2-hydroxypropan-2-yl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile;
2-(2-hydroxypropan-2-yl)-5-(2-(((3R,4S)-3-methyl-1-((1-methyl-1H-pyrazol-4-yl)sulfonyl) piperidin-4-yl)amino)-5-(trifluoromethyl)pyrimidin-4-yl)thiophene-3-carbonitrile;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-(2-hydroxypropan-2-yl)thiophene-3-carbonitrile;
5-(2-(((3R,4S)-1-((1H-imidazol-4-yl)sulfonyl)-3-methylpiperidin-4-yl)amino)-5-(trifluoromethyl)pyrimidin-4-yl)-2-(2-hydroxypropan-2-yl)thiophene-3-carbonitrile;
2-(2-hydroxypropan-2-yl)-5-(2-(((3R,4S)-3-methyl-1-((1-methyl-1H-imidazol-4-yl)sulfonyl) piperidin-4-yl)amino)-5-(trifluoromethyl)pyrimidin-4-yl)thiophene-3-carbonitrile;
5-(2-((1-(methylsulfonyl)piperidin-4-yl)amino)-5-(trifluoromethyl)pyrimidin-4-yl)-2-(2,2,2-trifluoro-1-hydroxyethyl)thiophene-3-carbonitrile;
5-(2-(((3R,4S)-3-methyl-1-((1-methyl-1H-pyrazol-4-yl)sulfonyl)piperidin-4-yl)amino)-5-(trifluoromethyl)pyrimidin-4-yl)-2-(2,2,2-trifluoro-1-hydroxyethyl)thiophene-3-carbonitrile;
2-(2-hydroxy-2-methylpropyl)-5-(2-((1-(methylsulfonyl)piperidin-4-yl)amino)-5-(trifluoromethyl)pyrimidin-4-yl)thiophene-3-carbonitrile;
2-(2-hydroxy-2-methylpropyl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carbonitrile;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carbonitrile;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)isothiazole-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)isothiazole-3-carboxamide;
2-(2-hydroxy-2-methylpropyl)-5-(2-((1-(methylsulfonyl)piperidin-4-yl)amino)-5-(trifluoromethyl)pyrimidin-4-yl)thiophene-3-carboxamide;
2-methyl-1-(2-(2-(((3R,4S)-3-methyl-1-(methylsulfonyl)piperidin-4-yl)amino)-5-(trifluoromethyl)pyrimidin-4-yl)thiazol-5-yl)propan-2-ol;
2-(2-(2-(((3R,4S)-3-methyl-1-(methylsulfonyl)piperidin-4-yl)amino)-5-(trifluoromethyl) pyrimidin-4-yl)thiazol-5-yl)propan-2-ol;
2-(2-(2-(((3R,4S)-3-methyl-1-((1-methyl-1H-imidazol-4-yl)sulfonyl)piperidin-4-yl)amino)-5-(trifluoromethyl)pyrimidin-4-yl)thiazol-5-yl)propan-2-ol;
2,2-difluoro-1-(2-(2-((1-(methylsulfonyl)piperidin-4-yl)amino)-5-(trifluoromethyl) pyrimidin-4-yl)thiazol-5-yl)ethan-1-ol;
1-(2-(2-(((3R,4R)-3-fluoro-1-(methylsulfonyl)piperidin-4-yl)amino)-5-(trifluoromethyl) pyrimidin-4-yl)thiazol-5-yl)-2-methylpropan-2-ol;
1-(2-(2-(((3R,4S)-3-fluoro-1-((1-methyl-1H-imidazol-4-yl)sulfonyl)piperidin-4-yl)amino)-5-(trifluoromethyl)pyrimidin-4-yl)thiazol-5-yl)-2-methylpropan-2-ol;
1-(2-(2-(((3R,4S)-1-((1H-pyrazol-4-yl)sulfonyl)-3-fluoropiperidin-4-yl)amino)-5-(trifluoromethyl)pyrimidin-4-yl)thiazol-5-yl)-2-methylpropan-2-ol;
2,2-difluoro-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)ethan-1-ol;
2,2-difluoro-1-(2-(2-(((3R,4S)-3-methyl-1-((1-methyl-1H-imidazol-4-yl)sulfonyl)piperidin-4-yl)amino)-5-(trifluoromethyl)pyrimidin-4-yl)thiazol-5-yl)ethan-1-ol;
2,2,2-trifluoro-1-(2-(2-(((3R,4S)-3-methyl-1-(methylsulfonyl)piperidin-4-yl)amino)-5-(trifluoromethyl)pyrimidin-4-yl)thiazol-5-yl)ethan-1-ol;
2,2,2-trifluoro-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)ethan-1-ol;
1,1-difluoro-2-(2-(2-(((3R,4S)-3-methyl-1-(methylsulfonyl)piperidin-4-yl)amino)-5-(trifluoromethyl)pyrimidin-4-yl)thiazol-5-yl)propan-2-ol;
1,1-difluoro-2-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
2-fluoro-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)ethan-1-ol;
2-fluoro-1-(2-(2-(((3R,4S)-3-methyl-1-(methylsulfonyl)piperidin-4-yl)amino)-5-(trifluoromethyl)pyrimidin-4-yl)thiazol-5-yl)ethan-1-ol;
2,2-difluoro-1-(2-(2-(((3R,4S)-3-methyl-1-(methylsulfonyl)piperidin-4-yl)amino)-5-(trifluoromethyl)pyrimidin-4-yl)thiazol-5-yl)ethan-1-ol;
2-methyl-1-(2-(2-(((3R,4S)-3-methyl-1-(morpholinosulfonyl)piperidin-4-yl)amino)-5-(trifluoromethyl)pyrimidin-4-yl)thiazol-5-yl)propan-2-ol;
2-methyl-1-(2-(2-(((3R,4S)-3-methyl-1-((4-methylpiperazin-1-yl)sulfonyl)piperidin-4-yl)amino)-5-(trifluoromethyl)pyrimidin-4-yl)thiazol-5-yl)propan-2-ol;
(3R,4S)-4-((4-(5-(2-hydroxy-2-methylpropyl)thiazol-2-yl)-5-(trifluoromethyl)pyrimidin-2-yl)amino)-N,N,3-trimethylpiperidine-1-sulfonamide;
1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)cyclopropan-1-ol;
1-(2-(2-(((3R,4S)-3-methyl-1-(methylsulfonyl)piperidin-4-yl)amino)-5-(trifluoromethyl) pyrimidin-4-yl)thiazol-5-yl)cyclopropan-1-ol;
1-(2-(5-chloro-2-(((3R,4S)-3-fluoro-1-(methylsulfonyl)piperidin-4-yl)amino)pyrimidin-4-yl)thiazol-5-yl)-2-methylpropan-2-ol;
2-(((3R,4S)-3-fluoro-1-(methylsulfonyl)piperidin-4-yl)amino)-4-(5-(2-hydroxy-2-methyl-propyl)thiazol-2-yl)pyrimidine-5-carbonitrile;
1-(2-(5-(difluoromethyl)-2-(((3R,4S)-3-fluoro-1-(methylsulfonyl)piperidin-4-yl)amino) pyrimidin-4-yl)thiazol-5-yl)-2-methylpropan-2-ol;
1-(2-(2-(((3R,4S)-3-fluoro-1-(methylsulfonyl)piperidin-4-yl)amino)-5-methylpyrimidin-4-yl)thiazol-5-yl)-2-methylpropan-2-ol;
   or a pharmaceutically acceptable salt thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

In some embodiments, the disclosure is directed to pharmaceutical compositions comprising compounds of Formula I, or a pharmaceutically acceptable salt or solvate thereof.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-1Ooleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%), 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)-aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; U.S. Pat. Nos. 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%> of a maximum effect in vivo.

In some embodiments, the subject methods utilize a CDK inhibitor with an IC50 value of about or less than a predetermined value, as ascertained in an in vitro assay. In some embodiments, the CDK inhibitor inhibits CDK a with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the CDK inhibitor selectively inhibits CDK a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other CDKs.

In some embodiments, the CDK inhibitor selectively inhibits CDK a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other CDKs.

The subject methods are useful for treating a disease condition associated with CDK. Any disease condition that results directly or indirectly from an abnormal activity or expression level of CDK can be an intended disease condition.

Different disease conditions associated with CDK have been reported. CDK has been implicated, for example, auto-immune diseases, neurodegeneration (such as Parkinson's disease, Alzheimer's disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

In some embodiments, the disclosure is directed to methods for treating a CDK4-mediated and a CDK6-mediated disorder in a patient in need thereof, comprising administering to said patient a compound of Formula I, including all subgenera described herein, or a pharmaceutically acceptable salt thereof, or a pharmaceutical composition comprising the compound of Formula I, including all subgenera described herein.

In some embodiments, the CDK4-mediated and CDK6-mediated disorder is a cancer. In some embodiments, the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

In some embodiments, the cancer is breast cancer. In some embodiments, the cancer is malignant brain tumors. In some embodiments, the cancer is colon cancer. In some embodiments, the cancer is small-cell lung cancer. In some embodiments, the cancer is non-small-cell lung cancer. In some embodiments, the cancer is bladder cancer. In some embodiments, the cancer is ovarian cancer.

In some embodiments, the cancer is prostate cancer. In some embodiments, the cancer is chronic lymphoid leukemia. In some embodiments, the cancer is lymphoma. In some embodiments, the cancer is myeloma. In some embodiments, the cancer is acute myeloid leukemia. In some embodiments, the cancer is secondary pancreatic cancer. In some embodiments, the cancer is secondary brain metastases.

In some embodiments, the breast cancer is HR+/HER2− or HR+/HER2+ advanced or metastatic breast cancer. In some embodiments, the breast cancer is HR+/HER2− advanced breast cancer. In some embodiments, the breast cancer is HR+/HER2− metastatic breast cancer. In some embodiments, the breast cancer is HR+/HER2+ advanced breast cancer. In some embodiments, the breast cancer is HR+/HER2+ metastatic breast cancer.

In some embodiments, the malignant brain tumors are glioblastoma, astrocytoma, or pontine glioma. In some embodiments, the malignant brain tumors are a glioblastoma. In some embodiments, the malignant brain tumors are an astrocytoma. In some embodiments, the malignant brain tumors are a pontine glioma.

In some embodiments, the patient is administered a pharmaceutical composition comprising a compound of Formula I, including all subgenera described herein, or a pharmaceutically acceptable salt thereof. In some embodiments, the administration is oral administration.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound of the invention can be administered in combination with a corticosteroid such as triamcinolone, dexamethasone, fluocinolone, cortisone, prednisolone, or flumetholone.

For treating autoimmune or inflammatory conditions, the compound of the invention can be administered in combination with an immune suppressant such as fluocinolone acetonide (Retisert®), rimexolone (AL-2178, Vexol, Alcon), or cyclosporine (Restasis®).

In some embodiments, the disclosure is directed to methods described herein, further comprising administering an additional therapeutic agent to the patient. In some embodiments, the additional therapeutic agent is a PRMT5 inhibitor, a HER2 kinase inhibitor, an aromatase inhibitor, an estrogen receptor antagonist or an alkylating agent.

In some embodiments, the additional therapeutic agent is a PRMT5 inhibitor. In some embodiments, the additional therapeutic agent is a HER2 kinase inhibitor. In other embodiments, the additional therapeutic agent is an aromatase inhibitor. In other embodiments, the additional therapeutic agent is an estrogen receptor antagonist. In yet other embodiments, the additional therapeutic agent is an alkylating agent.

In some embodiments, the aromatase inhibitor is letrozole. In some embodiments, the estrogen receptor antagonist is fulvestrant. In other embodiments, the alkylating agent is temozolomide.

In yet other embodiments, the PRMT5 inhibitor is a compound disclosed in US Published Patent Application No. 2020/0148692 (filed Jan. 16, 2020); US Published Patent Application No. 2019/0284193 (filed Apr. 5, 2019); and US Published Patent Application No. 2019/0048014 (filed Aug. 9, 2018); each of which is hereby incorporated herein in its entirety.

In some embodiments, the PRMT5 inhibitor is:
(2R,3R,4S,5S)-2-(4-amino-7H-pyrrolo[2,3-d]pyrimidin-7-yl)-5-((R)-6-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-7-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-5-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6,7-difluoroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-5,6-difluoroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6-chloro-5-fluoroisochroman-1-yl)tetrahydrofuran-3,4-diol, or a pharmaceutically acceptable salt or solvate thereof;
(2R,3R,4S,5S)-2-(4-amino-5-fluoro-7H-pyrrolo[2,3-d]pyrimidin-7-yl)-5-((R)-6-chloroisochroman-1-yl)tetrahydrofuran-3,4-diol, or a pharmaceutically acceptable salt or solvate thereof;
(2R,3R,4S,5S)-2-(4-amino-7H-pyrrolo[2,3-d]pyrimidin-7-yl)-5-((R)-6,7-dichloroisochroman-1-yl)tetrahydrofuran-3,4-diol, or a pharmaceutically acceptable salt or solvate thereof;
(2S,3S,4R,5R)-2-((R)-6-chloroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6,7-difluoroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-5,6-difluoroisochroman-1-yl)-5-(4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6-chloroisochroman-1-yl)-5-(5-fluoro-4-methyl-7H-pyrrolo[2,3-d]pyrimidin-7-yl)tetrahydrofuran-3,4-diol, or a pharmaceutically acceptable salt or solvate thereof;
(2S,3S,4R,5R)-2-((R)-6,7-dichloroisochroman-1-yl)-5-(4-methyl-7H-pyrrolo[2,3-d]pyrimidin-7-yl)tetrahydrofuran-3,4-diol, or a pharmaceutically acceptable salt or solvate thereof.

In some embodiments, the PRMT5 inhibitor is (2S,3S,4R,5R)-2-((R)-6-chloroisochroman-1-yl)-5-(4-methyl-7H-pyrrolo[2,3-d]pyrimidin-7-yl)tetrahydrofuran-3,4-diol, or a pharmaceutically acceptable salt or solvate thereof.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g., a reaction temperature, that is about the temperature of the room in which the reaction is carried out, for example, a temperature from about 20° C. to about 30° C.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

Compounds of Formula (I) can be prepared from optionally protected compounds 1-1 where X¹ is halogen (e.g., Cl, Br, or I) or pseudohalogen (e.g., OTf or OMs) as shown in Scheme 1. Compounds 1-1 can be coupled with compounds 1-2 where M¹ is a boronic acid, boronate ester, potassium trifluoroborate, or an appropriately substituted metal, such as Sn(Bu)₃, Sn(Me)₃, or ZnCl, under standard Suzuki conditions (e.g., in the presence of a palladium catalyst, such as [1,1′-bis(diphenylphosphino)ferrocene]dichloropalladium(II) and a base, such as K₃PO₄), or standard Stille conditions (e.g., in the presence of a palladium(0) catalyst, such as tetrakis(triphenyl-phosphine)palladium(0)), or standard Negishi conditions (e.g., in the presence of a palladium catalyst, such as tetrakis(triphenylphosphine)palladium(0) or [1,1′-bis(diphenylphosphino)ferrocene]dichloropalladium(II)), to give compounds 1-3 where X² is halogen (e.g., Cl, Br, or I) or pseudohalogen (e.g., OTf or OMs). Coupling of compounds 1-3 with amines 1-8, with appropriate protecting group (PG) (e.g., Boc, etc.), under standard Buchwald-Hartwig amination conditions (e.g., in the presence of a palladium catalyst, such as XPhos Pd G3, and a base, such as Cs₂CO₃ or K₃PO₄) or nucleophilic aromatic substitution conditions (e.g., in the presence of a base such as triethylamine) can provide compounds 1-9. Compounds 1-9 can be deprotected (e.g., TFA, etc.) and subsequently reacted with compounds 1-10 where X⁴ is halogen (e.g., F, C₁ or Br) under standard conditions (e.g., in the presence of a base, such as triethylamine) to afford compounds of Formula (I). Alternatively, compounds 1-3 can react with amines 1-11 under standard Buchwald-Hartwig amination conditions (e.g., in the presence of a palladium catalyst, such as XPhos Pd G3, and a base, such as Cs₂CO₃ or K₃PO₄) or nucleophilic aromatic substitution conditions (e.g., in the presence of a base such as triethylamine) to afford compounds of Formula (I).

Alternatively, compounds 1-1 can be converted to the appropriate compounds 1-4 (e.g., M² is B(OH)₂, Bpin, BF₃K, Sn(Me)₃, Sn(Bu)₃, or ZnCl) and then coupled to compounds 1-5 where X³ is halogen (e.g., Cl, Br, or I) or pseudohalogen (e.g., OTf or OMs) under standard Suzuki conditions (e.g., in the presence of a palladium catalyst, such as [1,1′-bis(diphenylphosphino) ferrocene]dichloropalladium(II), and a base, such as K₃PO₄) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine) palladium(0) or [1,1′-bis(diphenylphosphino)ferrocene]dichloropalladium(II)) to give compounds 1-3, which can be used to synthesize compounds of Formula (I).

Further alternatively, coupling of compounds 1-6 where X⁵ is halogen (e.g., Cl, Br, or I) or hydrogen with pyrimidines 1-7 in the presence of a strong base (such as LDA, BuLi etc.) and subsequent addition of DDQ can provide the appropriate intermediates 1-3 which can be converted to compounds of Formula (I).

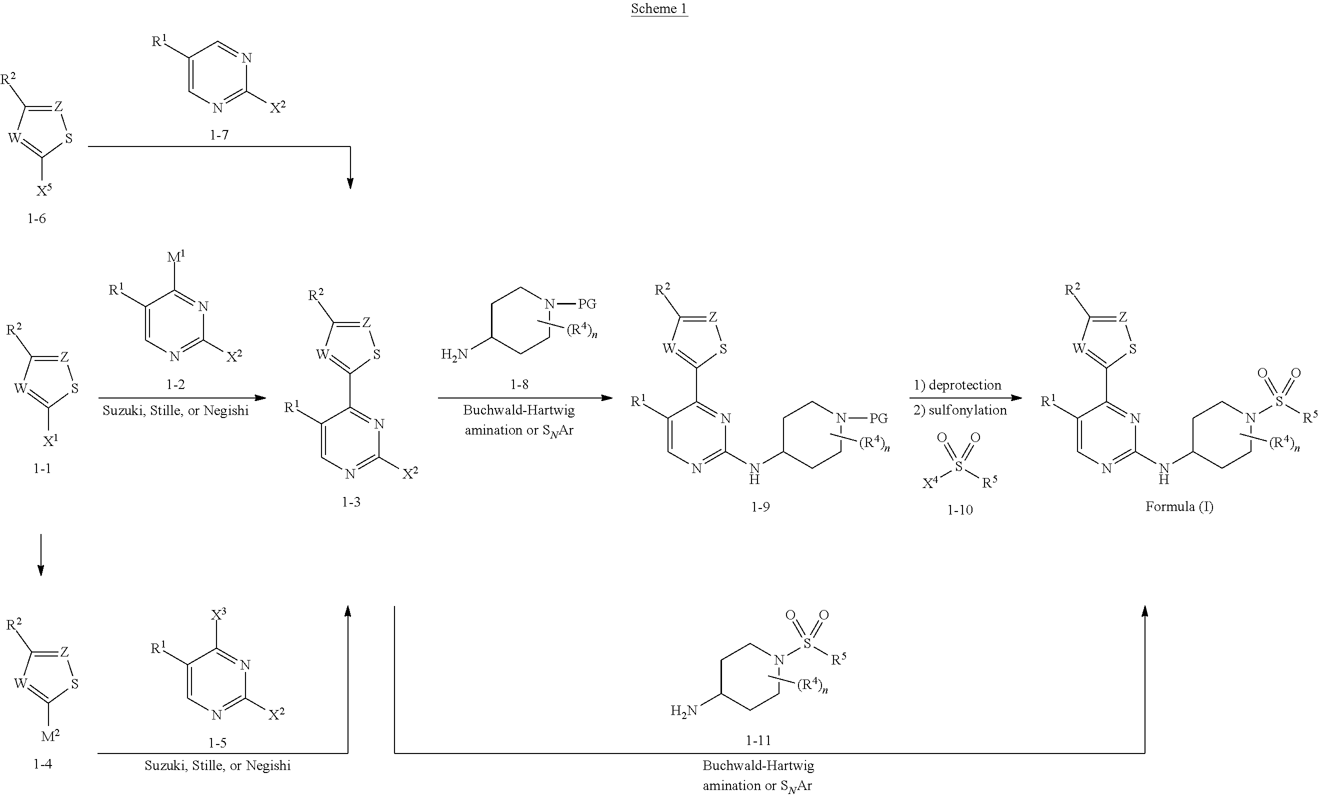

Compounds of Formula (I) can be prepared by the methods described in Scheme 2. Compounds 1-5 can react with amines 1-11 under amination conditions (e.g., in the presence of a Zn catalyst, such as ZnCl₂, and a base, such as Et₃N) to provide compounds 2-1. Compounds 2-1 can then be transformed to compounds of Formula (I) by reacting with compounds 1-4 under standard Suzuki conditions (e.g., in the presence of a palladium catalyst, such as [1,1′-bis(diphenylphosphino) ferrocene]dichloro-palladium(II), and a base, such as K₃PO₄), or standard Stille conditions (e.g., in the presence of a palladium(0) catalyst, such as tetrakis(triphenylphosphine)palladium(0)), or standard Negishi conditions (e.g., in the presence of a palladium(0) catalyst, such as tetrakis (triphenylphosphine) palladium(0) or [1,1′-bis(diphenylphosphino)ferrocene]dichloropalladium(II)). Alternatively, compounds 2-1 can be converted to the appropriate compounds 2-2 (e.g., M³ is B(OH)₂, Bpin, BF₃K, Sn(Me)₃, Sn(Bu)₃, or ZnCl) and then coupled to compounds 1-1 under standard Suzuki conditions (e.g., in the presence of a palladium catalyst, such as [1,1′-bis(diphenyl-phosphino)ferrocene]dichloropalladium(II), and a base, such as K₃PO₄) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenyl-phosphine)palladium(0) or [1,1′-bis(diphenylphosphino)ferrocene]dichloropalladium(II)) to synthesize compounds of Formula (I).

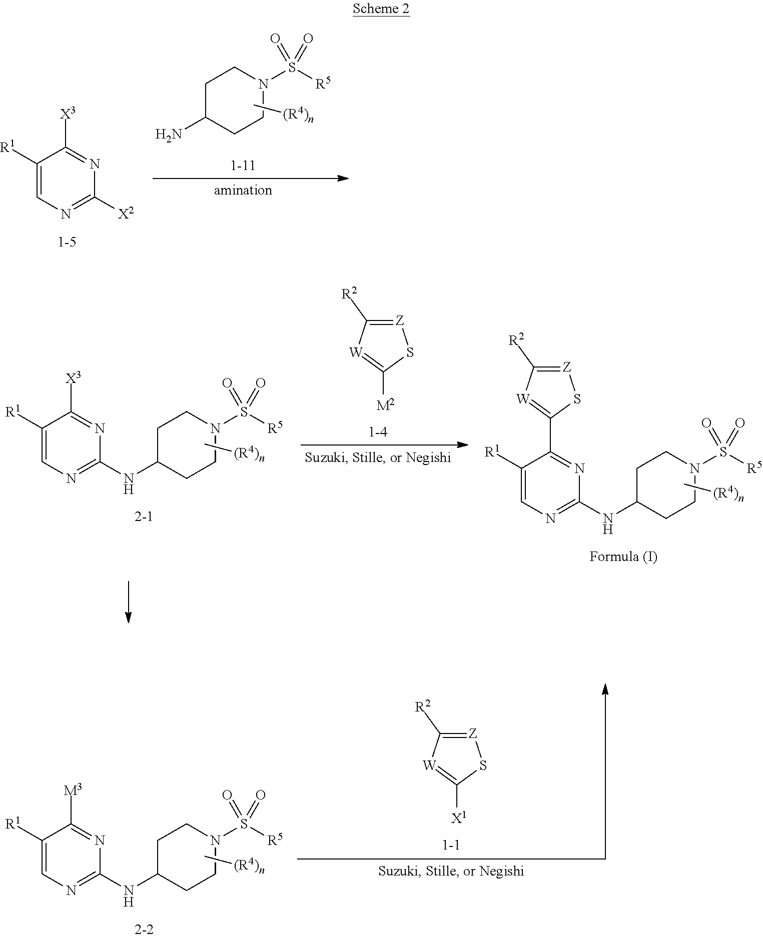

Intermediates for the synthesis of compounds of Formula (I) can be prepared as described in Scheme 3. Compounds 1-5 can react with amines 1-8 under amination conditions (e.g., in the presence of a Zn catalyst, such as ZnCl₂, and a base, such as Et₃N) to provide compounds of 3-1 which then can be transformed to compounds 1-9 by reacting with compounds 1-4 under standard Suzuki conditions (e.g., in the presence of a palladium catalyst, such as [1,1′-bis(diphenyl-phosphino)ferrocene]dichloro-palladium(II), and a base, such as K₃PO₄), or standard Stille conditions (e.g., in the presence of a palladium(0) catalyst, such as tetrakis(triphenylphosphine) palladium(0)), or standard Negishi conditions (e.g., in the presence of a palladium(0) catalyst, such as tetrakis(triphenylphosphine) palladium(0) or [1,1′-bis(diphenylphosphino)ferrocene]dichloropalladium(II)). Alternatively, compounds 3-1 can be converted to the appropriate compounds 3-2 (e.g., M⁴ is B(OH)₂, Bpin, BF₃K, Sn(Me)₃, Sn(Bu)₃, or ZnCl) and then coupled to compounds 1-1 under standard Suzuki conditions (e.g., in the presence of a palladium catalyst, such as [1,1′-bis(diphenylphosphino)ferrocene]dichloropalladium(II), and a base, such as K₃PO₄) or standard Stille conditions (e.g., in the presence of a palladium(0) catalyst, such as tetrakis(triphenyl-phosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium(II)) to synthesize compounds 1-9.

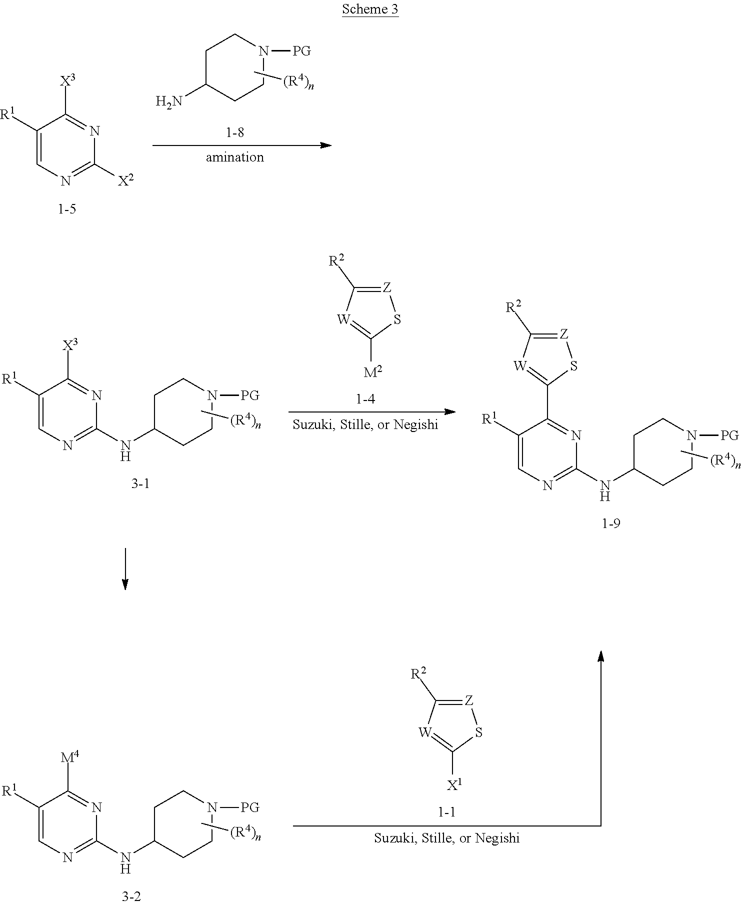

Intermediates for the synthesis of compounds of Formula (I) can be prepared as described in Scheme 4. Compounds 4-1 can be halogenated with suitable reagents, such as N-bromo-succinimide or N-iodosuccinimide, to provide compounds 1-1. Alternatively, compounds 4-1 can be metalated in the presence of a strong base, such as lithium diisopropylamide or butyllithium, and an appropriate reagent (e.g., 2-isopropoxy-4,4,5,5-tetramethyl-1,3,2-dioxaborolane, hexamethylditin, trimethyltin chloride, or zinc chloride) to afford compounds 1-4.

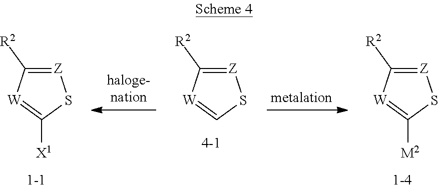

Intermediates for the synthesis of compounds of Formula (I) can be prepared as described in Scheme 5. Grignard reactions of esters 5-1 and Grignard reagent can provide alcohols 5-2. Compounds 5-2 can be converted into diazonium salts by treatment with tert-butyl nitrite or sodium nitrite in the presence of an acid, followed by standard Sandmeyer reaction conditions (e.g., CuCl, CuBr, CuI, etc.) to afford compounds 5-3.

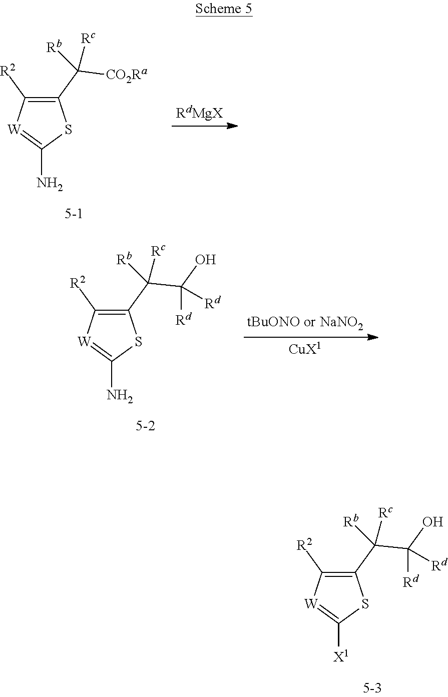

Intermediates for the synthesis of compounds of Formula (I) can be prepared as described in Scheme 6. Lithiation reactions of compounds 6-1 with strong base such as n-butyllithium or lithium diisopropylamide, in the presence of an ester or Weinreb amide can afford ketones 6-2. Next Grignard reactions of compounds 6-2 can provide alcohols 6-3.

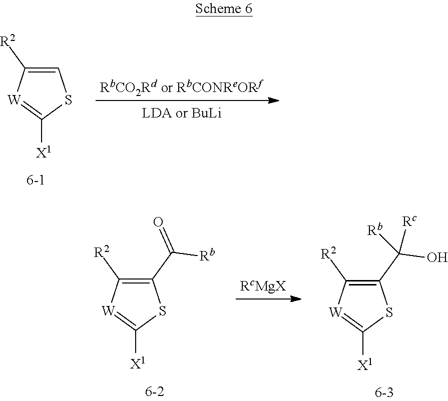

Compounds of Formula (II) can be prepared as described in Scheme 7. Compounds 5-3 can be coupled with compounds 1-2 under standard Suzuki conditions (e.g., in the presence of a palladium catalyst, such as [1,1′-bis(diphenylphosphino)ferrocene]dichloropalladium(II) and a base, such as K₃PO₄), or standard Stille conditions (e.g., in the presence of a palladium(0) catalyst, such as tetrakis(triphenyl-phosphine)palladium(0)), or standard Negishi conditions (e.g., in the presence of a palladium catalyst, such as tetrakis(triphenylphosphine)palladium(0) or [1,1′-bis(diphenylphosphino)ferrocene]dichloropalladium(II)), to give compounds 7-1. Coupling of compounds 7-1 with amines 1-8 under standard Buchwald-Hartwig amination conditions (e.g., in the presence of a palladium catalyst, such as XPhos Pd G3, and a base, such as Cs₂CO₃ or K₃PO₄) or nucleophilic aromatic substitution conditions (e.g., in the presence of a base such as triethylamine) can provide compounds 7-2. Compounds 7-2 can be deprotected (e.g., TFA, etc.) and subsequently reacted with compounds 1-10 under standard conditions (e.g., in the presence of a base, such as triethylamine) to afford compounds of Formula (II).

Alternatively, coupling of compounds 5-3 where X¹ is halogen (e.g., Cl, Br, or I) with pyrimidines 1-7 in the presence of a strong base (such as LDA, BuLi etc.) and subsequent addition of DDQ can provide the appropriate intermediates 7-1 which can be converted to compounds of Formula (II).

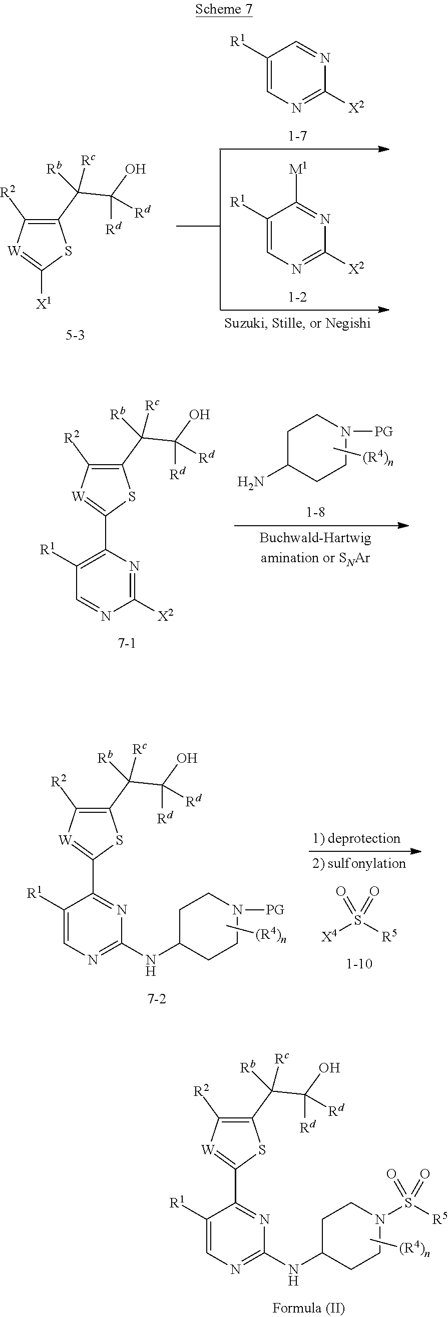

### Intermediate 1. 5-Bromo-2-(1-hydroxyethyl)thiophene-3-carbonitrile

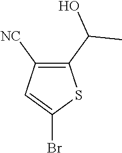

### Step 1. 2-(1-Hydroxyethyl)thiophene-3-carbonitrile

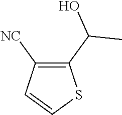

A cold solution of thiophene-3-carbonitrile (1.00 g, 9.16 mmol) in THF (20.0 mL) at −78° C. under N₂ was added n-BuLi (4.76 mL, 11.9 mmol) dropwise. The reaction was stirred at −78° C. for 20 mins and was quenched by addition of acetaldehyde (2.57 mL, 45.8 mmol). The reaction was slowly warmed to RT. The HPLC and LC-MS showed full consumption of starting material. The reaction was poured into cold NH₄Cl solution and was extracted (3×EtOAc). The combined organic phase was dried over Na₂SO₄ and filtered. After removal of solvent, the residue was purified by silica gel column chromatography eluted by acetyl acetate/heptanes 5-60% to give 2-(1-hydroxyethyl)thiophene-3-carbonitrile (1.05 g, 74.8% yield). ¹H NMR (300 MHz, CDCl₃) δ 7.31-7.25 (m, 1H), 7.16 (dd, J=5.2, 1.9 Hz, 1H), 5.50-5.28 (m, 2H), 1.65 (dd, J=6.4, 2.8 Hz, 3H).

### Step 2. 5-Bromo-2-(1-hydroxyethyl)thiophene-3-carbonitrile

A solution of 2-(1-hydroxyethyl)thiophene-3-carbonitrile (1.10 g, 7.18 mmol) in anhydrous THF (30.0 mL) at −78° C. was added n-BuLi (6.61 mL, 16.5 mmol) dropwise under N₂. The reaction was stirred for 20 mins at −78° C. then CBr₄ (2.86 g, 8.62 mmol) in 5.00 mL of THF was added. The reaction was further stirred at −78° C. for 30 mins then was slowly warmed to 0° C. The reaction turned black and the HPLC showed the conversion rate >95%. The reaction was poured into cold NH₄Cl solution and was extracted (3×EtOAc). The combined organic phase was dried over Na₂SO₄. After removal of solvent, the residue was purified by silica gel column chromatography eluted by acetyl acetate/heptanes 0-25% to give 5-bromo-2-(1-hydroxyethyl)-thiophene-3-carbonitrile (1.26 g, 75.6% yield). ¹H NMR (300 MHz, CDCl₃) δ 7.10 (s, 1H), 5.34 (q, J=6.4 Hz, 1H), 2.63 (s, 1H), 1.63 (d, J=8.4 Hz, 3H).

### Intermediate 2. 5-Bromoisothiazole-3-carboxamide

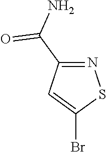

### Step 1. 5-Bromoisothiazole-3-carboxamide

A cold solution of 5-bromo-1,2-thiazole-3-carboxylic acid (105.0 mg, 0.500 mmol) in DCM (2.00 mL) was added thionyl chloride (3.00 mL, 41.3 mmol) at 0° C. The reaction was stirred at 75° C. for 1 h. The HPLC showed full consumption of stating material. The solvent was removed and was dissolved in 3.00 mL of MTBE and was added to cold concentrated ammonia solution. The reaction mixture was stirred at RT for 20 mins then was extracted by EtOAc×3. The combined organic phase was dried over Na₂SO₄ and filtered. After removal of solvent, the residue was purified by silica gel column chromatography eluted by acetyl acetate/heptanes 20-50% to give 5-bromoisothiazole-3-carboxamide (92.0 mg, 88.0% yield). ¹H NMR (300 MHz, DMSO-d₆) δ 8.05 (s, 1H), 7.88 (s, 1H), 7.73 (s, 1H).

### Intermediate 3. 5-Bromo-2-(1-hydroxyethyl)thiophene-3-carboxamide

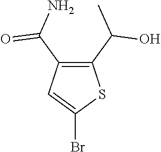

### Step 1. Thiophene-3-carboxamide

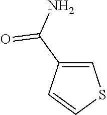

3-Thiophenecarboxylic acid (2.56 g, 20.0 mmol) in SOCl₂ (20.0 mL) was refluxed at 85° C. After 30 min, the mixture turned clear. The reaction was cooled to RT then concentrated to give the acyl chloride. Next, the acyl chloride intermediate in anhydrous dichloromethane (10.0 mL) was added dropwise to a solution of ammonia (aq) (con. 30.0 mL) on ice. After stirring for 30 min, the precipitate was collected by suction filtration, washed with water and n-hexane, then dried under reduced pressure at 40° C. A slurry from ethyl acetate afforded the thiophene-3-carboxamide (1.80 g, 70.8% yield). LCMS calculated for C₅H₆NOS (M+H)⁺: m/z=128.01; Found 128.0. ¹H NMR (300 MHz, DMSO-d₆) δ 8.13 (dd, J=2.9, 1.3 Hz, 1H), 7.79 (s, 1H), 7.55 (dd, J=5.0, 3.0 Hz, 1H), 7.48 (dd, J=5.0, 1.3 Hz, 1H), 7.23 (s, 1H).

### Step 2. 2-(1-Hydroxyethyl)thiophene-3-carboxamide

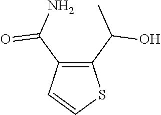

To a stirred solution of the thiophene-3-carboxamide (200 mg, 1.57 mmol) in THF (5.00 mL) was added TMEDA (0.710 mL, 4.72 mmol) and n-BuLi (1.89 mL, 4.72 mmol) dropwise at −78° C. while stirring under N₂ atmosphere. The reaction mixture was stirred at −78° C. temperature for 1 h then treated with acetaldehyde (0.440 mL, 7.86 mmol). The resulting mixture was warmed to RT, then quenched with cold NH₄Cl solution and extracted 3× with chloroform/IPA (v/v=3/1). The extracts were dried over Na₂SO₄, filtered, and evaporated. After removal of solvent, the residue was purified by silica gel column chromatography eluted by acetyl acetate/heptanes 10-80% to give 2-(1-hydroxyethyl)thiophene-3-carboxamide (205 mg, 76.1% yield). LCMS calculated for C₇H₈NOS [MS−OH]⁺: m/z=154.0; Found 154.0. ¹H NMR (300 MHz, CDCl₃) δ 7.21 (d, J=5.3 Hz, 1H), 7.16 (d, J=5.3 Hz, 1H), 6.45 (s, 1H), 6.13 (s, 1H), 5.34 (d, J=4.5 Hz, 1H), 5.31-5.19 (m, 1H), 1.65 (d, J=6.5 Hz, 3H).

### Step 3. 5-Bromo-2-(1-hydroxyethyl)thiophene-3-carboxamide

To a solution of 2-(1-hydroxyethyl)thiophene-3-carboxamide (140.0 mg, 0.820 mmol) in MeCN (3.00 mL) was added NBS (175 mg, 0.980 mmol). The reaction was stirred at RT for 8 mins. The solution turned yellow and was immediately purified by prep-HPLC on C₁₈ column (30×250 mm, 10 m) using mobile phase 10% to 55% MeCN/H₂O (w/0.1% TFA) (tR=18 min). The desired fractions were collected, neutralized by NaHCO₃ solution to pH-7, concentrated and extracted 2× with chloroform/IPA (v/v=3/1). The combined organic phase was dried over Na₂SO₄, filtered and condensed to give 5-bromo-2-(1-hydroxyethyl)thiophene-3-carboxamide (128 mg, 62.6% yield) as a white solid. LCMS calculated for C₇H₇BrNOS [MS−OH]⁺: m/z=233.9/231.9; Found 233.9/231.9. ¹H NMR (300 MHz, DMSO-d₆) δ 7.66 (s, 1H), 7.45 (s, 1H), 7.33 (s, 1H), 5.93 (d, J=4.0 Hz, 1H), 5.51 (qd, J=6.2, 4.2 Hz, 1H), 1.34 (d, J=6.2 Hz, 3H).

### Intermediate 4. 5-Bromo-2-(2-hydroxy-2-methylpropyl)thiophene-3-carboxamide

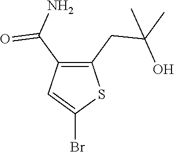

### Step 1. 2-(2-Hydroxy-2-methylpropyl)thiophene-3-carboxylic acid

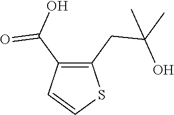

A solution of 2-methylthiophene-3-carboxylic acid (1.00 g, 7.03 mmol) in THF (20.0 mL) under N₂ was added LDA (7.74 mL, 15.4 mmol) at −40° C. dropwise. The reaction was stirred at −40° C. for 1 h then acetone (0.780 mL, 10.6 mmol) was added. The reaction was further stirred at −40° C. for 2 h and was slowly warmed to RT. The HPLC showed two products formed with a ratio of 3:1. The reaction was quenched by cold NH₄Cl solution and was adjusted to pH 2-3 with 1N HCl(aq). The mixture was then extracted by MTBE (3×). The combined organic phase was washed by brine, dried over Na₂SO₄, filtered and condensed. The residue was purified by silica gel column chromatography eluted by MTBE/toluene 5-50% to give 2-(2-hydroxy-2-methylpropyl)thiophene-3-carboxylic acid (825 mg, 58.6% yield). ¹H NMR (300 MHz, CDCl₃) δ 7.48 (d, J=5.4 Hz, 1H), 7.14 (d, J=5.4 Hz, 1H), 3.43 (s, 2H), 1.32 (s, 6H).

### Step 2. 6,6-Dimethyl-6,7-dihydro-4H-thieno[3,2-c]pyran-4-one

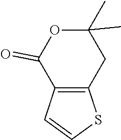

A mixture of 2-(2-hydroxy-2-methylpropyl)thiophene-3-carboxylic acid (2.40 g, 12.0 mmol), EDC (4.60 g, 24.0 mmol), DMAP (1.46 g, 12.0 mmol) and Et₃N (5.01 mL, 36.0 mmol) in DCE (30.0 mL) was stirred at 40° C. for 3 h. The LC-MS showed full consumption of starting material. The reaction was diluted by DCM and was poured into 1 N HCl solution. The mixture was extracted by DCM (2×). The combined organic phase was washed with brine, dried over Na₂SO₄, and condensed. The residue was purified by silica gel column chromatography eluted by acetyl acetate/heptanes 0-20% to give 6,6-dimethyl-6,7-dihydro-4H-thieno[3,2-c]pyran-4-one (1.30 g, 59.5% yield). LCMS calculated for C₉H₁₁O₂S (M+H)⁺: m/z=183.05; Found 183.1. ¹H NMR (300 MHz, CDCl₃) δ 7.45 (d, J=5.2 Hz, 1H), 7.15 (d, J=5.2 Hz, 1H), 3.11 (s, 2H), 1.52 (s, 6H).

### Step 3. 2-(2-Hydroxy-2-methylpropyl)thiophene-3-carboxamide

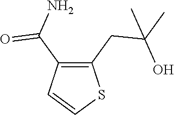

A mixture of 6,6-dimethyl-6,7-dihydro-4H-thieno[3,2-c]pyran-4-one (920 mg, 5.05 mmol) in 18.0 mL of conc. ammonia (aq) stirred at 80° C. for 5 h in a sealed tube. The LCMS showed full consumption of starting material. The reaction was cooled to 0° C. and was poured into NH₄Cl solution. The mixture was extracted 3× with chloroform/IPA (v/v=3/1). The combined organic phase was dried over Na₂SO₄, filtered and concentrated to give 2-(2-hydroxy-2-methylpropyl)thiophene-3-carboxamide (672 mg, 66.8% yield) as a white solid and was used in the next step without further purification. LCMS calculated for C₉H₁₂NO_{S} [MS−OH]⁺: m/z=182.06; Found 182.1.

### Step 4. 5-Bromo-2-(2-hydroxy-2-methylpropyl)thiophene-3-carboxamide

A mixture of 2-(2-hydroxy-2-methylpropyl)thiophene-3-carboxamide (620 mg, 3.11 mmol) in MeCN (20.0 mL) was added NBS (609 mg, 3.42 mmol). The reaction was stirred at RT for 1 h. The LCMS showed full consumption of starting material. The reaction was concentrated and poured into brine. The mixture was extracted 3× with chloroform/IPA (v/v=3/1). The combined organic phase was dried over Na₂SO₄, filtered and condensed. The residue was purified by silica gel column chromatography eluted by acetyl acetate/heptanes 10-60% to give 5-bromo-2-(2-hydroxy-2-methylpropyl)thiophene-3-carboxamide (805 mg, 93.0% yield) as a white solid containing succinimide as an impurity. LCMS calculated for C₉H₁₁BrNOS [MS−OH]⁺: m/z=259.97/261.97; Found 259.9/261.9. ¹H NMR (300 MHz, CDCl₃) δ 7.19 (s, 1H), 6.99 (s, 1H), 5.91 (s, 1H), 3.70 (s, 1H), 3.17 (s, 2H), 1.35 (s, 6H).

### Intermediate 5. 5-Bromo-2-(2-hydroxy-2-methylpropyl)thiophene-3-carbonitrile

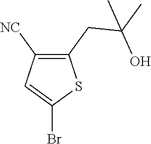

### Step 1. 5-Bromo-2-(2-hydroxy-2-methylpropyl)thiophene-3-carbonitrile

A solution of 5-bromo-2-(2-hydroxy-2-methylpropyl)thiophene-3-carboxamide (200 mg, 0.500 mmol, 69% purity) in anhydrous DMF (6.00 mL) at −10° C. was added a cold solution of phosphorous oxychloride (90.0 μL, 0.990 mmol) in 0.500 mL of DMF. The reaction was stirred for 30 mins. The LCMS and HPLC showed the conversion rate >95%. The reaction was directly purified by prep-HPLC on C₁₈ column (30×250 mm, 10 μm) using mobile phase 10% to 60% MeCN/H₂O (w/0.1% TFA) (tR=18 min). The desired fractions were collected, concentrated, poured into NaHCO₃(sat) solution and was extracted by DCM (3×). The combined organic phase was dried over Na₂SO₄, filtered and condensed to give 5-bromo-2-(2-hydroxy-2-methylpropyl)thiophene-3-carbonitrile (62.0 mg, 48.0% yield) as colorless oil. LCMS calculated for C₉H₁₁BrNOS (M+H)⁺: m/z=259.97/261.97; Found 260.0/262.0. ¹H NMR (300 MHz, CDCl₃) δ 7.11 (s, 1H), 3.10 (s, 2H), 1.31 (s, 6H).

### Intermediate 6. 1-(2-Bromothiazol-5-yl)ethan-1-ol

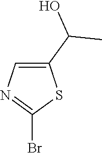

### Step 1. 1-(2-Bromothiazol-5-yl)ethan-1-ol

To a solution of 2-bromo-1,3-thiazole-5-carbaldehyde (1.00 g, 5.20 mmol) in THF (10.0 mL) was added methylmagnesium bromide (2.60 mL, 3.0 M in Et₂O, 7.80 mmol) dropwise at 0° C. The reaction mixture was stirred at 0° C. for 30 min. LCMS showed the starting material was consumed. The reaction was quenched with saturated NH₄Cl solution (10.0 mL) and extracted with EtOAc (10.0 mL×3). The combined organic layers were dried over Na₂SO₄, filtered and concentrated. The residue was purified by silica gel chromatography eluting with EtOAc/heptane (0-40%) to yield 1-(2-bromothiazol-5-yl)ethan-1-ol (640 mg, 59.1% yield). ¹H NMR (300 MHz, CDCl₃) δ 7.40 (d, J=0.8 Hz, 1H), 5.19-5.07 (m, 1H), 2.35 (d, J=4.8 Hz, 1H), 1.60 (d, J=6.4 Hz, 3H). LCMS calculated for C₅H₇BrNOS (M+H)⁺: m/z=207.94/209.94; Found 208.0/210.0.

### Intermediate 7. 1-(2-Bromothiazol-5-yl)-2-methylpropan-2-ol

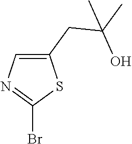

### Step 1. 1-(2-aminothiazol-5-yl)-2-methylpropan-2-ol

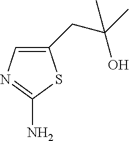

To a solution of methylmagnesium bromide (0.780 mL, 3.0 M in Et₂O, 2.30 mmol) in THF (1.00 mL) was added a solution of ethyl 2-(2-amino-1,3-thiazol-5-yl)acetate (87.0 mg, 0.470 mmol) in THF (1.00 mL) dropwise at 0° C. The reaction mixture was stirred at 0° C. 30 min. LCMS showed the starting material was consumed. The reaction was quenched with saturated NH₄Cl solution (2.00 mL) and extracted with EtOAc (3.00 mL×3). The combined organic layers were dried over Na₂SO₄, filtered and concentrated. The residue was purified by silica gel chromatography eluting with EtOAc/heptane (0-100%) to yield 1-(2-aminothiazol-5-yl)-2-methylpropan-2-ol (53.0 mg, 65.9% yield). LCMS calculated for C₇H₁₃N₂OS (M+H)⁺: m/z=173.07; found 173.1.

### Step 2. 1-(2-bromothiazol-5-yl)-2-methylpropan-2-ol

To a mixture of tert-butyl nitrite (63.0 mg, 0.620 mmol) and copper(II) bromide (137 mg, 0.620 mmol) in MeCN (1.00 mL) was added 1-(2-aminothiazol-5-yl)-2-methylpropan-2-ol (53.0 mg, 0.310 mmol). The reaction mixture was stirred at RT for 1 h. LCMS showed the starting material was consumed. The reaction was diluted with EtOAc (2.00 mL) and washed with 1N HCl solution (2.00 mL), dried over Na₂SO₄, filtered and concentrated. The residue was purified by silica gel chromatography eluting with EtOAc/heptane (0-25%) to yield 1-(2-bromothiazol-5-yl)-2-methylpropan-2-ol (21.0 mg, 28.9% yield). ¹H NMR (300 MHz, CDCl₃) δ 7.31 (s, 1H), 2.93 (s, 2H), 1.27 (s, 6H). LCMS calculated for C₇H₁₁BrNOS (M+H)⁺: m/z=235.97/237.97; Found 236.0/238.0.

### Intermediate 8. 1-(2-Bromothiazol-5-yl)-2,2,2-trifluoroethan-1-ol

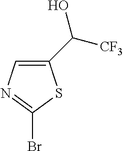 [SMILES: OC(c1cnc(Br)s1)C(F)(F)F]

### Step 1. 1-(2-Bromothiazol-5-yl)-2,2,2-trifluoroethan-1-one

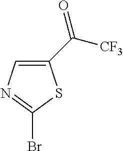 [SMILES: O=C(c1cnc(Br)s1)C(F)(F)F]

To a solution of 2-bromo-1,3-thiazole (1.14 g, 6.95 mmol) and ethyl 2,2,2-trifluoroacetate (1.48 g, 10.4 mmol) in THF (15.0 mL) was added lithium diisopropylamide (5.20 mL, 2.0 M in THF/heptane/ethylbenzene 10.4 mmol) dropwise at −78° C. The reaction mixture was stirred at −78° C. for 30 min. LCMS showed the starting material was consumed. The reaction was quenched with saturated NH₄Cl solution (10.0 mL) and extracted with EtOAc (10.0 mL×3). The combined organic layers were dried over Na₂SO₄, filtered and concentrated. The residue was purified by silica gel chromatography eluting with EtOAc/heptane (0-30%) to yield 1-(2-bromothiazol-5-yl)-2,2,2-trifluoroethan-1-one (975 mg, 53.9% yield). LCMS calculated for C₅H₄BrF₃NO₂S (M+H₂O+H)⁺: m/z=277.91/279.91; Found 277.9/279.9.

### Step 2. 1-(2-Bromothiazol-5-yl)-2,2,2-trifluoroethan-1-ol

To a solution of 1-(2-bromothiazol-5-yl)-2,2,2-trifluoroethan-1-one (700 mg, 2.69 mmol) in THF (7.00 mL) was added sodium borohydride (204 mg, 5.38 mmol) at 0° C. The reaction mixture was stirred at 0° C. for 1 h. LCMS showed the starting material was consumed. The reaction was quenched with 1N HCl solution and concentrated. The residue was dissolved in DCM (5.00 mL) and washed with water (5.00 mL) and brine (5.00 mL), dried over Na₂SO₄, filtered and concentrated. The residue was purified by silica gel chromatography eluting with EtOAc/heptane (0-20%) to yield 1-(2-bromothiazol-5-yl)-2,2,2-trifluoroethan-1-ol (460 mg, 65.2% yield). ¹H NMR (300 MHz, CDCl₃) δ 7.63 (s, 1H), 5.32 (q, J=6.2 Hz, 1H), 3.44 (d, J=30.8 Hz, 1H). LCMS calculated for C₅H₄BrF₃NOS (M+H)⁺: m/z=261.91/263.91; Found 261.9/264.0.

### Intermediate 9. 5-Bromo-3-(difluoromethyl)-2-methylthiophene

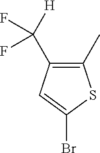

### Step 1. 5-Bromo-2-methylthiophene-3-carbaldehyde

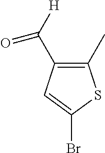

To a solution of (5-bromo-2-methylthiophen-3-yl)methanol (130 mg, 0.628 mmol) in DCM (2.00 mL) was added Dess-Martin Periodinane (399 mg, 0.942 mmol). The reaction mixture was stirred at RT for 1 h. LCMS showed the starting material was consumed. The reaction was quenched with saturated NaHCO₃ solution (1.00 mL) and 10% Na₂S₂O₃ (1.00 mL) and stirred at RT for 30 min. The layer was separated, and the aqueous layer was extracted with DCM (2.00 mL×3). The combined organic layers were dried over Na₂SO₄, filtered and concentrated. The residue was purified by silica gel chromatography eluting with EtOAc/heptane (0-10%) to yield 5-bromo-2-methylthiophene-3-carbaldehyde (103 mg, 80.0% yield). 1H NMR (300 MHz, CDCl₃) δ 9.90 (s, 1H), 7.33 (s, 1H), 2.72 (s, 3H).

### Step 2. 5-Bromo-3-(difluoromethyl)-2-methylthiophene

To a solution of diethylaminosulfur trifluoride (118 mg, 0.731 mmol) in DCM (0.500 mL) was added a solution of 5-bromo-2-methylthiophene-3-carbaldehyde (50.0 mg, 0.243 mmol) in DCM (0.500 mL) dropwise at −78° C. The reaction mixture was stirred from −78° C. to RT overnight. LCMS showed the starting material was consumed. The reaction was quenched with saturated NaHCO₃ solution (2.00 mL) and extracted with DCM (2.00 mL×3). The combined organic layers were dried over Na₂SO₄, filtered and concentrated. The residue was purified by silica gel chromatography eluting with EtOAc/heptane (0-20%) to yield 5-bromo-3-(difluoromethyl)-2-methylthiophene (38.1 mg, 68.6% yield). 1H NMR (300 MHz, CDCl₃) δ 7.04 (s, 1H), 6.60 (t, J=55.5 Hz, 1H), 2.46 (t, J=2.0 Hz, 3H).

### Intermediate 10. 5-Bromothiophene-3-carboxamide

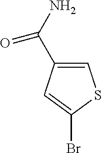

### Step 1: 5-bromothiophene-3-carboxamide

To a solution of thiophene-3-carboxamide (300 mg, 2.42 mmol) in MeCN (10.0 mL) was added N-bromosuccinimide (415 mg, 2.42 mmol). The mixture was stirred at rt for 12 h. LCMS showed that the starting material was consumed. The solvent was removed under reduced pressure. The crude was then added H₂O (50.0 mL) and extracted with ethyl acetate (50.0 mL×3). The combined organic layers were dried over Na₂SO₄, filtered, and concentrated. The crude was purified by prep-HPLC on C₁₈ column (10-60% MeCN in 0.1% TFA(aq), pH=2) to afford the title compound (176 mg, 35.9% yield). LCMS calc. for C₅H₅BrNOS [M+H]⁺: m/z=205.9/207.9; Found 205.4/207.4.

### Intermediate 11. 5-Bromothiophene-3-carbonitrile

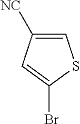

### Step 1. 5-Bromothiophene-3-carbonitrile

To a solution of 5-bromothiophene-3-carboxamide (60.0 mg, 0.320 mmol) in DMF (3.00 mL) was added POCl₃ (312 mg, 2.01 mmol). The solution was then stirred at rt for 3 h. LCMS showed that the starting material was consumed. The reaction was then added H₂O (3 mL) and extracted with EA/heptane (1:1, 5 mL×3). The combined organic layers were dried over Na₂SO₄, filtered, and concentrated to provide the title compound (42.0 mg, 76.7% yield).

### Intermediate 12. 5-Bromo-2-methylthiophene-3-carboxylic acid

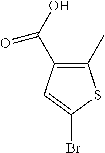

### Step 1. 5-Bromo-2-methylthiophene-3-carboxylic acid

To a solution of 2-methylthiophene-3-carboxylic acid (1.00 g, 7.04 mmol) in MeCN (10.0 mL) was added N-bromosuccinimide (1.30 g, 7.41 mmol). The mixture was stirred at rt for 12 h. LCMS showed that the starting material was consumed. The solvent was removed under reduced pressure. The crude was then added H₂O (50.0 mL) and extracted with ethyl acetate (50.0 mL×3). The combined organic layers were dried over Na₂SO₄, filtered, concentrated to provide the title compound (1.30 g, 5.91 mmol, 83.6% yield). The resulting solid was used in the next step without further purification. LCMS calc. for C₆H₆BrO₂S [M+H]⁺: m/z=220.9/222.9; Found 220.8/222.8.

### Intermediate 13. 5-Bromo-2-methylthiophene-3-carboxamide

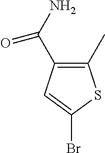

### Step 1. 5-Bromo-2-methylthiophene-3-carboxamide

A solution of 5-bromo-2-methylthiophene-3-carboxylic acid (350 mg, 1.58 mmol) in thionyl chloride (3.28 mL, 45.1 mmol) was stirred at 80° C. for 30 min. The solution was then co-evaporated with toluene (20.0 mL×3) under reduced pressure. The resulting residue was then diluted with DCM (1.00 mL). The solution was then dropwise added to ice-cold NH₄OH (10 mL, 25% aq.), and stirred for 30 min until fully precipitated. The resulting mixture was then filtered, washed with H₂O (10.0 mL×2) and heptane (10.0 mL×2). The solid was then dried to provide the title compound (310 mg, 88.9% yield). LCMS calc. for C₆H₇BrNOS [M+H]⁺: m/z=219.9/221.9; Found 219.8/221.8.

### Intermediate 14. 5-Bromo-2-methylthiophene-3-carbonitrile

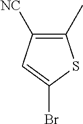

### Step 1: 5-bromo-2-methylthiophene-3-carbonitrile

To a solution of 5-bromo-2-methylthiophene-3-carboxamide (378 mg, 1.72 mmol) in DMF (3.00 mL) was added POCl₃ (1.84 g, 12.0 mmol). The solution was then stirred at rt for 3 h. LCMS showed that the starting material was consumed. The reaction crude was then slowly added to ice water (10.0 mL). The precipitate was then filtered and dried to provide the title compound (309 mg, 89.3% yield).

### Intermediate 15. 1-(5-Bromo-2-methylthiophen-3-yl)ethenone

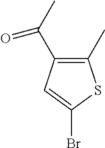

### Step 1. 5-Bromo-N-methoxy-N,2-dimethylthiophene-3-carboxamide

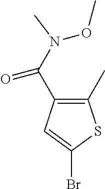

To a solution of 5-bromo-2-methylthiophene-3-carboxylic acid (300 mg, 1.36 mmol) in DCM (5.00 mL) were added N-methoxymethanamine (91.2 mg, 1.49 mmol), 1-hydroxy-benzotriazole hydrate (9.20 mg, 70.0 μmol), 1-ethyl-3-(3-dimethylaminopropyl)-carbodiimide hydrochloride (520 mg, 2.71 mmol) and DIPEA (701 mg, 5.43 mmol). The reaction was then stirred at rt for 12 h. LCMS showed that the starting material was consumed. The solvent was then removed under reduced pressure. To the crude was added H₂O (15.0 mL) and then was extracted with ethyl acetate (15.0 mL×3). The combined organic layers were dried over Na₂SO₄, filtered, and concentrated. The residue was purified by flash column chromatography (0-50% EtOAc/heptane) to provide the title compound (249 mg, 69.5% yield).

### Step 2. 1-(5-Bromo-2-methylthiophen-3-yl)ethenone

To a solution of 5-bromo-N-methoxy-N,2-dimethylthiophene-3-carboxamide (249 mg, 0.940 mmol) in THF (3.00 mL) was added methylmagnesium chloride solution (141 mg, 1.89 mmol) dropwise at −78° C. The reaction was then stirred at rt for 4 h. LCMS showed that the starting material was consumed. The reaction was then poured into ice cold NH₄Cl (10 mL, 10% aq.) and extracted with ethyl acetate (5.00 mL×3). The combined organic layers were dried over Na₂SO₄, filtered, concentrated to provide the title compound (185 mg, 89.6% yield). ¹H NMR (300 MHz, CDCl₃) δ 7.30 (s, 1H), 2.67 (s, 3H), 2.46 (s, 3H).

### Intermediate 16. 5-Bromo-3-(methoxymethyl)-2-methylthiophene

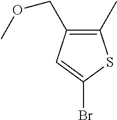

### Step 1. (5-Bromo-2-methylthiophen-3-yl)methanol

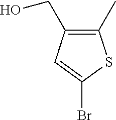

To a solution of 5-bromo-2-methylthiophene-3-carboxylic acid (500 mg, 2.26 mmol) in THF (5.00 mL) was added borane dimethyl sulfide complex (515 mg, 6.79 mmol) dropwise at 0° C. The reaction was then stirred at rt for 12 h. LCMS showed that the starting material was consumed completely. The reaction was then quenched with MeOH (30.0 mL) slowly. The solvent was then removed under reduced pressure. The residue was purified by flash column chromatography (0-50% EtOAc/heptane) to provide the title compound (440 mg, 93.9% yield). LCMS calc. for C₆H₈BrOS [M+H]⁺: m/z=206.9/208.9; Found 206.8/208.9.

### Step 2. 5-Bromo-3-(methoxymethyl)-2-methylthiophene

To a solution of (5-bromo-2-methylthiophen-3-yl)methanol (150 mg, 0.720 mmol) in DMF (2.00 mL) was added NaH (26.1 mg, 1.09 mmol, 60% suspension in mineral oil) at 0° C. The mixture was stirred for 15 min, followed by the addition of Mel (154 mg, 1.09 mmol). The mixture was then stirred at rt for 3 h. LCMS showed that the starting material was consumed. To the reaction was then added H₂O (3.00 mL) and was then extracted with EA/heptane (1:1, 5.00 mL×3). The combined organic layers were dried over Na₂SO₄, filtered, and concentrated. The residue was purified by flash column chromatography (0-30% EtOAc/heptane) to provide the title compound (140 mg, 87.4% yield).

### Intermediate 17. 5-Bromo-3-methoxy-2-methylthiophene

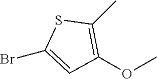

### Step 1. 3-Methoxy-2-methylthiophene

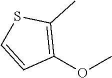

A solution of 3-methoxythiophene (2.00 g, 17.5 mmol) in THF (20.0 mL) was added n-BuLi (9.11 mL, 22.8 mmol) in hexane at 0° C. under N₂. The reaction was stirred at 35° C. for 40 mins. Dimethyl sulfate (1.74 mL, 18.4 mmol) was added at 0° C. dropwise. The reaction was stirred at 35° C. for 30 mins. The HPLC showed a major new peak. To the reaction was added 6.00 mL of conc. NH₄OH solution at 0° C. and stirred at RT for 30 mins. The reaction was extracted with MTBE (3×). The combined organic phase was dried over Na₂SO₄, filtered and condensed. The residue was purified by silica gel column chromatography with DCM to give 3-methoxy-2-methyl-thiophene (1.80 g, 80.2% yield). ¹H NMR (300 MHz, CDCl₃) δ 6.96 (d, J=5.5 Hz, 1H), 6.80 (d, J=5.5 Hz, 1H), 3.82 (s, 3H), 2.28 (s, 3H).

### Step 2. 5-Bromo-3-methoxy-2-methylthiophene

A solution of 3-methoxy-2-methylthiophene (100.0 mg, 0.780 mmol) in THF (3.00 mL) was added n-BuLi (0.340 mL, 0.860 mmol) in hexane at 0° C. under N₂. The reaction was stirred 35° C. for 40 mins then CBr₄ (776 mg, 2.34 mmol) was added in 1.00 mL of THF at 0° C. The reaction was stirred at 35° C. for 30 mins. HPLC showed most of the starting material was consumed. The reaction was poured into Na₂S₂O₃ solution and extracted by DCM (2×). The combined organic phase was dried over Na₂SO₄, filtered, and condensed. The residue was purified by silica gel column chromatography eluted by heptanes to give 5-bromo-3-methoxy-2-methylthiophene (63.0 mg, 39.0% yield) as a yellow oil. ¹H NMR (300 MHz, CDCl₃) δ 6.78 (s, 1H), 3.77 (s, 3H), 2.20 (s, 3H).

### Intermediate 18. tert-Butyl (3R,4S)-4-((4-chloro-5-(trifluoromethyl)pyrimidin-2-yl)amino)-3-fluoropiperidine-1-carboxylate

### Step 1: Tert-butyl (3R,4S)-4-((4-chloro-5-(trifluoromethyl)pyrimidin-2-yl)amino)-3-fluoropiperidine-1-carboxylate

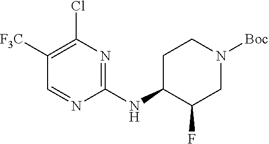 [SMILES: CC(C)(C)OC(=O)N1CC[C@H](Nc2ncc(C(F)(F)F)c(Cl)n2)[C@H](F)C1]

To a solution of 2,4-dichloro-5-(trifluoromethyl)pyrimidine (260 μL, 1.93 mmol) in tert-butyl alcohol (3.80 mL) and 1,2-dichloroethane (3.80 mL) was added ZnCl₂ solution (1.9 M in 2-MeTHF, 1.45 mL, 2.74 mmol) at 0° C. The resulting mixture was stirred at 0° C. for one hour. Next tert-butyl (3R,4S)-4-amino-3-fluoropiperidine-1-carboxylate (400 mg, 1.83 mmol) was added to the reaction, followed by triethylamine (308 μL, 2.22 mmol). The resulting mixture was stirred at 45° C. overnight. The reaction mixture was concentrated to dryness and diluted with acetonitrile (10.0 mL). The mixture was centrifuged to remove any undissolved solids and the remaining solution was purified by prep-HPLC on a C₁₈ column (20-80% MeCN in 0.1% TFA(aq), pH=2). The fractions of sufficient purity were basified with saturated NaHCO₃ (aq) and collected to afford the title compound (279 mg, 0.700 mmol, 38.0% yield). ¹H NMR (300 MHz, CDCl₃) δ 8.45 (s, 1H), 5.90-5.80 (m, 1H), 4.85-4.70 (m, 1H), 4.50-4.40 (m, 1H), 4.30-4.20 (m, 1H), 3.15-2.80 (m, 2H), 1.90-1.80 (m, 2H), 1.48 (s, 9H).

### Intermediate 19. Tert-butyl (3R,4R)-4-((4-chloro-5-(trifluoromethyl)pyrimidin-2-yl)amino)-3-fluoropiperidine-1-carboxylate

### Step 1. Tert-butyl (3R,4R)-4-((4-chloro-5-(trifluoromethyl)pyrimidin-2-yl)amino)-3-fluoropiperidine-1-carboxylate

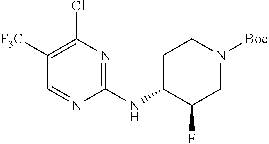 [SMILES: CC(C)(C)OC(=O)N1CC[C@@H](Nc2ncc(C(F)(F)F)c(Cl)n2)[C@H](F)C1]

To a solution of 2,4-dichloro-5-(trifluoromethyl)pyrimidine (292 μL, 2.17 mmol) in tert-butyl alcohol (4.30 mL) and 1,2-dichloroethane (4.30 mL) was added ZnCl₂ solution (1.9 M in 2-MeTHF, 1.60 mL, 3.08 mmol) at 0° C. The resulting mixture was stirred at 0° C. for one hour. Next tert-butyl (3R,4R)-4-amino-3-fluoropiperidine-1-carboxylate (450 mg, 2.06 mmol) was added to the reaction, followed by triethylamine (350 μL, 2.50 mmol). The resulting mixture was stirred at 45° C. overnight. The reaction mixture was concentrated to dryness and diluted with acetonitrile (10.0 mL). The mixture was centrifuged to remove any undissolved solids and the remaining solution was purified by prep-HPLC on a C₁₈ column (20-80% MeCN in 0.1% TFA(aq), pH=2). The fractions of sufficient purity were basified with saturated NaHCO₃ (aq) and collected to afford the title compound (347 mg, 0.870 mmol, 42.0% yield). ¹H NMR (300 MHz, CDCl₃) δ 8.46 (s, 1H), 5.65-5.60 (m, 1H), 4.50-4.25 (m, 3H), 4.00-3.90 (m, 1H), 3.10-3.00 (m, 2H), 2.25-2.00 (m, 1H), 1.48 (s, 9H).

### Intermediate 20: Tert-butyl 4-((4-chloro-5-(trifluoromethyl)pyrimidin-2-yl)amino)piperidine-1-carboxylate

### Step 1. Tert-butyl 4-((4-chloro-5-(trifluoromethyl)pyrimidin-2-yl)amino)piperidine-1-carboxylate

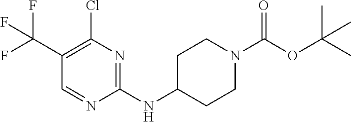 [SMILES: CC(C)(C)OC(=O)N1CCC(Nc2ncc(C(F)(F)F)c(Cl)n2)CC1]

To a solution of 2,4-dichloro-5-(trifluoromethyl)pyrimidine (25.0 g, 115 mmol) in DCE (200 mL)/tert-butanol (200 mL) was added zinc chloride (23.5 g, 173 mmol) at 0° C. The mixture was stirred for 1 h, followed by the dropwise addition of 4-amino-1-boc-piperidine (23.0 g, 115 mmol) and triethylamine (17.5 g, 173 mmol) at 0° C. The mixture was then stirred at 40° C. overnight. LCMS showed that the starting material was consumed. The solvent was removed under reduced pressure. The mixture was then added H₂O (200 mL) and extracted with ethyl acetate (200 mL×3). The combined organic layers were dried over Na₂SO₄, filtered, concentrated. The crude was then slurry washed in MeOH (20 mL) for 30 min, filtered to provide pure title compound (10.5 g, 23.4% yield). LCMS calc. for C₁₅H₂₁ClF₃N₄O₂ [M+H]⁺: m/z=381.1; Found 381.1.

### Intermediate 21. 4-Chloro-N-(piperidin-4-yl)-5-(trifluoromethyl)pyrimidin-2-amine

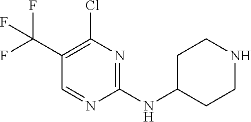 [SMILES: FC(F)(F)c1cnc(NC2CCNCC2)nc1Cl]

### Step 1. 4-Chloro-N-(piperidin-4-yl)-5-(trifluoromethyl)pyrimidin-2-amine

To a stirred mixture of tert-butyl 4-[[4-chloro-5-(trifluoromethyl)pyrimidin-2-yl]amino]piperidine-1-carboxylate (Intermediate 20) (1200.0 mg, 3.15 mmol) in DCM (2.5 mL) was added TFA (1.0 mL, 13.07 mmol) dropwise. The mixture was stirred for 1 h. LCMS analysis indicated the reaction was complete. The crude product obtained was used directly without further purification. LCMS calc. for C₁₀H₁₃ClF₃N₄[M+H]⁺: m/z=281.1; Found 281.2.

### Intermediate 22. 1-((1-Methyl-1H-pyrazol-4-yl)sulfonyl)piperidin-4-amine

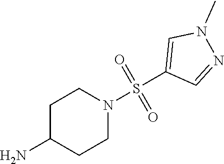 [SMILES: Cn1cc(S(=O)(=O)N2CCC(N)CC2)cn1]

### Step 1. Tert-butyl (1-((1-methyl-1H-pyrazol-4-yl)sulfonyl)piperidin-4-yl)carbamate

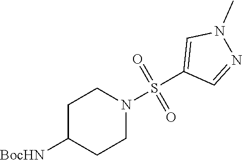 [SMILES: Cn1cc(S(=O)(=O)N2CCC(NC(=O)OC(C)(C)C)CC2)cn1]

To a solution of 4-(N-Boc-amino)piperidine (200 mg, 0.999 mmol) and N,N-diisopropyl-ethylamine (0.430 mL, 2.50 mmol) in DCM (5.00 mL) was added 1-methylpyrazole-4-sulfonyl chloride (216 mg, 1.20 mmol) at 0° C. The reaction mixture was stirred at RT overnight. LCMS showed the starting material was consumed. The reaction was diluted with DCM (5.00 mL) and washed with saturated NaHCO₃ solution (5.00 mL) and brine (5.00 mL), dried over Na₂SO₄, filtered and concentrated. The residue was purified by silica gel chromatography eluting with EtOAc/heptane (0-100%) to yield tert-butyl (1-((1-methyl-1H-pyrazol-4-yl)sulfonyl) piperidin-4-yl)carbamate (310 mg, 90.1% yield). LCMS calculated for C₁₄H₂₅N₄O₄S (M+H)⁺: m/z=345.16; Found 345.1.

### Step 2. 1-((1-Methyl-1H-pyrazol-4-yl)sulfonyl)piperidin-4-amine

To a solution of tert-butyl (1-((1-methyl-1H-pyrazol-4-yl)sulfonyl)piperidin-4-yl) carbamate (310 mg, 0.900 mmol) in DCM (1.00 mL) was added trifluoroacetic acid (1.00 mL, 13.0 mmol). The reaction mixture was stirred at RT for 1 h. LCMS showed the starting material was consumed. The solvent was removed under reduced pressure. The residue was lyophilized to 1-((1-methyl-1H-pyrazol-4-yl)sulfonyl)piperidin-4-amine (200 mg, 91.0% yield), which was used for the next reaction without further purification. LCMS calculated for C₉H₁₇N₄O₂S (M+H)⁺: m/z=245.11; Found 245.1.

### Intermediate 23. Tert-butyl 4-((5-(trifluoromethyl)-4-(trimethylstannyl)pyrimidin-2-yl)amino)piperidine-1-carboxylate

### Step 1. Tert-butyl 4-((5-(trifluoromethyl)-4-(trimethylstannyl)pyrimidin-2-yl)amino) piperidine-1-carboxylate

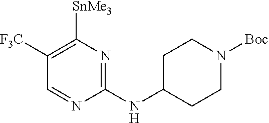

To a tube was added tert-butyl 4-((4-chloro-5-(trifluoromethyl)pyrimidin-2-yl)amino) piperidine-1-carboxylate (Intermediate 20) (1.0 g, 2.62 mmol), hexamethylditin (806 μL, 3.94 mmol), dichlorobis(triphenylphosphine)palladium(II) (184 mg, 0.262 mmol), and dioxane (13.0 mL). The tube was sealed, and the reaction mixture was degassed by sparging with nitrogen. The reaction mixture was stirred overnight at 100° C. After cooling to rt, the reaction mixture was filtered off any undissolved solids and the resulting clear solution was used in the next step without further purification. LCMS calc. for C₁₈H₃₀F₃N₄O₂Sn [M+H]⁺: m/z=511.1; found 510.9.

### Intermediate 24. 4-Chloro-N-(1-((1-methyl-1H-pyrazol-4-yl)sulfonyl)piperidin-4-yl)-5-(trifluoromethyl)pyrimidin-2-amine

### Step 1. 4-Chloro-N-(1-((1-methyl-1H-pyrazol-4-yl)sulfonyl)piperidin-4-yl)-5-(trifluoro-methyl)pyrimidin-2-amine

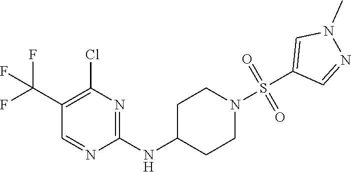 [SMILES: Cn1cc(S(=O)(=O)N2CCC(Nc3ncc(C(F)(F)F)c(Cl)n3)CC2)cn1]

To a solution of 4-chloro-N-piperidin-4-yl-5-(trifluoromethyl)pyrimidin-2-amine (Intermediate 21) (800 mg, 2.83 mmol) in DMA (10.0 mL) was dropwise added 1-methyl-pyrazole-4-sulfonyl chloride (766 mg, 4.24 mmol) at 0° C., followed by the addition of DIPEA (1.10 g, 8.52 mmol). The mixture was then stirred at rt for 1 h. LCMS showed that the starting material was consumed. The residue was purified by prep-HPLC on a C₁₈ column (10-80% MeCN in 0.1% TFA(aq), pH=2) to yield the title compound (640 mg, 53.3% yield). LCMS calc. for C₁₄H₁₇ClF₃N₆O₂S [M+H]⁺: m/z=425.1; Found 425.0.

### Intermediate 25. Tert-butyl (3R,4S)-4-((4-chloro-5-(trifluoromethyl)pyrimidin-2-yl)amino)-3-methylpiperidine-1-carboxylate

### Step 1. Tert-butyl (3R,4S)-4-((4-chloro-5-(trifluoromethyl)pyrimidin-2-yl)amino)-3-methylpiperidine-1-carboxylate

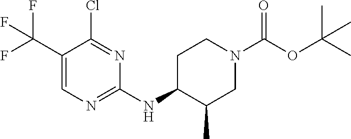 [SMILES: C[C@@H]1CN(C(=O)OC(C)(C)C)CC[C@@H]1Nc1ncc(C(F)(F)F)c(Cl)n1]

To a solution of 2,4-dichloro-5-(trifluoromethyl)pyrimidine (1.10 g, 4.73 mmol) in DCE (12.0 mL)/tert-butanol (12.0 mL) was added zinc chloride (890 mg, 6.52 mmol) at 0° C. The mixture was stirred for 1 h, followed by the dropwise addition of tert-butyl (3R,4S)-4-amino-3-methylpiperidine-1-carboxylate (1.00 g, 4.73 mmol) and triethylamine (538 mg, 5.31 mmol) at 0° C. The mixture was then stirred at 40° C. overnight. LCMS showed that the starting material was consumed. The solvent was removed under reduced pressure. The mixture was then added H₂O (10.0 mL) and extracted with ethyl acetate (10 mL×3). The combined organic layers were dried over Na₂SO₄, filtered, concentrated to provide a mixture of isomers. The crude was purified by prep-HPLC on C₁₈ column (15-70% MeCN in 0.1% TFA(aq), pH=2) to afford the title compound (1.10 g, 2.78 mmol, 59.7% yield). LC-MS calc. for C₁₆H₂₃ClF₃N₆O₂ [M+H]⁺: m/z=395.1; Found 395.1.

### Intermediate 26. 4-Chloro-N-(1-(methylsulfonyl)piperidin-4-yl)-5-(trifluoromethyl)pyrimidin-2-amine

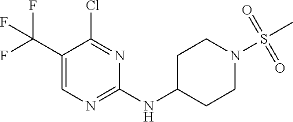 [SMILES: CS(=O)(=O)N1CCC(Nc2ncc(C(F)(F)F)c(Cl)n2)CC1]

### Step 1. 4-Chloro-N-(1-(methylsulfonyl)piperidin-4-yl)-5-(trifluoromethyl)pyrimidin-2-amine

A mixture of 2,4-dichloro-5-(trifluoromethyl)pyrimidine (10.0 g, 46.09 mmol) in tert-butanol (100 mL) and DCE (100 mL) was cooled to 0° C. in an ice bath before 1 M zinc chloride solution (69.13 mL, 69.13 mmol) in diethyl ether was added and the resulting mixture was purged with nitrogen and stirred at 0° C. for 1 h. To the reaction mixture was then added 1-methylsulfonyl-piperidin-4-amine; 2,2,2-trifluoroacetic acid (12.8 g, 43.78 mmol) followed by dropwise addition of TEA (9.64 mL, 69.13 mmol). The ice bath was then removed, the reaction mixture was allowed to warm to RT before heating to 60° C. overnight. After cooling to RT, the reaction mixture was then concentrated and purified by silica gel column chromatography eluting with 0 to 50% EA/hep. The collected fractions were purified by prep-HPLC eluting with 10% to 80% ACN/H₂O (including 0.1% TFA). The desired fractions were collected to yield (5.1 g, 32.5% yield) target compound as white solid. LCMS calc. for C₁₁H₁₅ClF₃N₄O₂S [M+H]⁺: m/z=359.1; Found: 358.8. 1H NMR (300 MHz, Chloroform-d) δ 8.45 (s, 1H), 5.54 (s, 1H), 4.11-3.95 (m, 1H), 3.79 (d, J=11.2 Hz, 2H), 2.97-2.85 (m, 2H), 2.82 (s, 3H), 2.16 (d, J=10.9 Hz, 2H), 1.71-1.62 (m, 3H).

### Intermediate 27. N-(1-methylsulfonylpiperidin-4-yl)-5-(trifluoromethyl)-4-trimethylstannyl-pyrimidin-2-amine

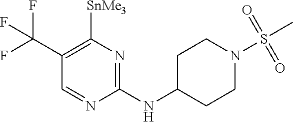

### Step 1. N-(1-methylsulfonylpiperidin-4-yl)-5-(trifluoromethyl)-4-trimethylstannyl-pyrimidin-2-amine

To a solution of 4-chloro-N-(1-methylsulfonylpiperidin-4-yl)-5-(trifluoromethyl) pyrimidin-2-amine (Intermediate 26) (72.0 mg, 0.201 mmol) and hexamethylditin (0.0624 mL, 0.301 mmol) in 1,4-dioxane (1.00 mL) was added bis(triphenylphosphine)palladium(II) dichloride (14.1 mg, 0.0201 mmol). The reaction mixture was bubbled with N₂ for 1 min and stirred at 100° C. overnight. LCMS showed the starting material was consumed. The solution was filtered and concentrated to yield N-(1-methylsulfonylpiperidin-4-yl)-5-(trifluoromethyl)-4-trimethylstannyl-pyrimidin-2-amine (88 mg, 89.7% yield), which was used for the next reaction without further purification. LCMS calc. for C₁₄H₂₄F₃N₄O₂SSn [M+H]⁺: m/z=489.06; found 488.9.

### Intermediate 28. 4-Chloro-N-[1-(1-methylpyrazol-4-yl)sulfonylpiperidin-4-yl]-5-(trifluoromethyl)pyrimidin-2-amine

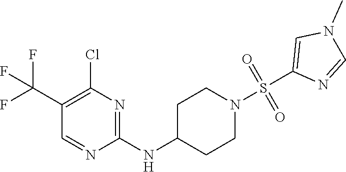 [SMILES: Cn1cnc(S(=O)(=O)N2CCC(Nc3ncc(C(F)(F)F)c(Cl)n3)CC2)c1]

### Step 1. 4-Chloro-N-[1-(1-methylpyrazol-4-yl)sulfonylpiperidin-4-yl]-5-(trifluoromethyl) pyrimidin-2-amine

To a solution of 4-chloro-N-piperidin-4-yl-5-(trifluoromethyl)pyrimidin-2-amine (Intermediate 21) (2.7 g, 9.62 mmol) in DMA (20 mL) was added 1-methyl-1H-imidazole-4-sulfonyl chloride (1.9 g, 10.58 mmol) at 0° C., followed by the addition of DIPEA (1.2 g, 96.19 mmol). The mixture was then stirred at RT for 3 h. LC-MS showed the starting material was consumed. The crude was purified by prep-HPLC on a C₁₈ column (10-80% MeCN in 0.1% TFA(aq), pH=2) to yield the title compound (2.0 g, 46.8% yield). LCMS calc. for C₁₄H₁₇ClF₃N₆O₂S [M+H]⁺: m/z=425.1; Found 424.8.

### Intermediate 29. Tert-butyl (3R,4R)-3-fluoro-4-[[5-(trifluoromethyl)-4-trimethylstannyl-pyrimidin-2-yl]amino]piperidine-1-carboxylate

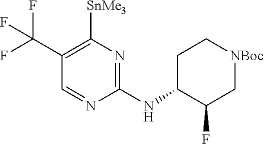

### Step 1. Tert-butyl (3R,4R)-3-fluoro-4-[[5-(trifluoromethyl)-4-trimethylstannylpyrimidin-2-yl]amino]piperidine-1-carboxylate

To a solution of tert-butyl (3R,4R)-4-[[4-chloro-5-(trifluoromethyl)pyrimidin-2-yl]amino]-3-fluoropiperidine-1-carboxylate (Intermediate 19) (104.0 mg, 0.261 mmol) and hexamethylditin (0.0811 mL, 0.391 mmol) in 1,4-dioxane (1.20 mL) was added bis(triphenyl-phosphine)palladium(II) dichloride (18.3 mg, 0.0261 mmol). The reaction mixture was bubbled with N₂ for 1 min and stirred at 100° C. overnight. LCMS showed the starting material was consumed. The solution was filtered and concentrated to yield tert-butyl (3R,4R)-3-fluoro-4-[[5-(trifluoro-methyl)-4-trimethylstannylpyrimidin-2-yl]amino]piperidine-1-carboxylate (133 mg, 97.0% yield), which was used for the next reaction without further purification. LCMS calc. for C₁₈H₂₉F₄N₄O₂Sn [M+H]⁺: m/z=529.12; found 528.8.

### Intermediate 30: Tert-butyl (3R,4S)-3-fluoro-4-((5-(trifluoromethyl)-4-(trimethylstannyl)-pyrimidin-2-yl)amino)piperidine-1-carboxylate

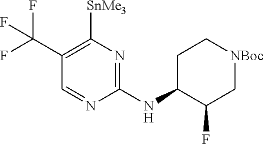

### Step 1. Tert-butyl (3R,4S)-3-fluoro-4-((5-(trifluoromethyl)-4-(trimethylstannyl)pyrimidin-2-yl)amino)piperidine-1-carboxylate

Tert-butyl (3R,4S)-4-((4-chloro-5-(trifluoromethyl)pyrimidin-2-yl)amino)-3-fluoro-piperidine-1-carboxylate (Intermediate 18) (300 mg, 0.752 mmol), hexamethylditin (230 μL, 1.13 mmol), dichlorobis(triphenylphosphine)palladium(II) (53 mg, 0.075 mmol) and dioxane (3.8 mL) were added to a tube. The tube was sealed, and the resulting mixture was degassed by sparging nitrogen. The reaction mixture was stirred at 90° C. overnight. The mixture was cooled to room temperature and filtered of any undissolved solids. The resulting solution was used as is in the next step without further purification. LCMS calc. for C₁₈H₂₉F₄N₄O₂Sn [M+H]⁺: m/z=529.1; found 528.8.

### Intermediate 31. Tert-butyl (3R,4S)-3-methyl-4-((5-(trifluoromethyl)-4-(trimethylstannyl)-pyrimidin-2-yl)amino)piperidine-1-carboxylate

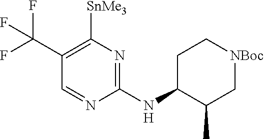

### Step 1. Tert-butyl (3R,4S)-3-methyl-4-((5-(trifluoromethyl)-4-(trimethylstannyl)pyrimidin-2-yl)amino)piperidine-1-carboxylate

Tert-butyl (3R,4S)-4-((4-chloro-5-(trifluoromethyl)pyrimidin-2-yl)amino)-3-methyl-piperidine-1-carboxylate (Intermediate 25) (75 mg, 0.19 mmol), hexamethylditin (58 μL, 0.29 mmol), dichlorobis(triphenylphosphine)palladium(II) (13 mg, 0.019 mmol) and dioxane (0.95 mL) were added to a tube. The tube was sealed, and the resulting mixture was degassed by sparging nitrogen. The reaction mixture was stirred at 90° C. overnight. The mixture was cooled to room temperature and filtered of any undissolved solids. The resulting solution was used as is in the next step without further purification. LCMS calc. for C₁₉H₃₂F₃N₄O₂Sn [M+H]⁺: m/z=525.1; found 525.1.

### Intermediate 32. N-((3R,4S)-1-((1H-imidazol-4-yl)sulfonyl)-3-methylpiperidin-4-yl)-4-chloro-5-(trifluoromethyl)pyrimidin-2-amine

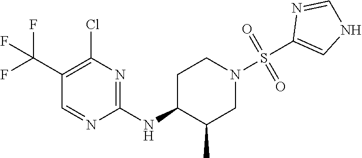 [SMILES: C[C@@H]1CN(S(=O)(=O)c2c[nH]cn2)CC[C@@H]1Nc1ncc(C(F)(F)F)c(Cl)n1]

### Step 1. Tert-butyl ((3R,4S)-1-((1H-imidazol-4-yl)sulfonyl)-3-methylpiperidin-4-yl)carbamate

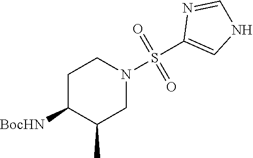 [SMILES: C[C@@H]1CN(S(=O)(=O)c2c[nH]cn2)CC[C@@H]1NC(=O)OC(C)(C)C]

To a solution of tert-butyl N-[(3R,4S)-3-methylpiperidin-4-yl]carbamate (90.0 mg, 0.42 mmol) in DCM (3.00 mL) was added 1H-imidazole-4-sulfonyl chloride (69.9 mg, 0.42 mmol) at 0° C., followed by the addition of DIPEA (109 mg, 0.84 mmol). The mixture was then stirred at RT for 3 h. LCMS showed the starting material was consumed. The solvent was removed under reduced pressure to afford tert-butyl N-[(3R,4S)-1-(1H-imidazol-4-ylsulfonyl)-3-methylpiperidin-4-yl]carbamate (140 mg, 90.3% yield), which was used directly in next step without further purification. LCMS calc. for C₁₄H₂₅N₄O₄S [M+H]⁺: m/z=345.16; found 344.9.

### Step 2. (3R,4S)-1-((1H-imidazol-4-yl)sulfonyl)-3-methylpiperidin-4-amine

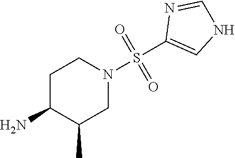 [SMILES: C[C@@H]1CN(S(=O)(=O)c2c[nH]cn2)CC[C@@H]1N]

The solution of tert-butyl N-[(3R,4S)-1-(1H-imidazol-4-ylsulfonyl)-3-methylpiperidin-4-yl]carbamate (130.0 mg, 0.35 mmol) in 4 N HCl in dioxane (2.00 mL) was stirred at r.t. for 1 h. LC-MS showed that the starting material was consumed. The solvent was then removed under reduced pressure to afford (3R,4S)-1-(1H-imidazol-4-ylsulfonyl)-3-methylpiperidin-4-amine (90 mg, quant. yield), which is directly used in the next step without further purification. LCMS calc. for C₉H₁₇N₄O₂S [M+H]⁺: m/z=245.11; found 245.1.

### Step 3. N-((3R,4S)-1-((1H-imidazol-4-yl)sulfonyl)-3-methylpiperidin-4-yl)-4-chloro-5-(trifluoromethyl)pyrimidin-2-amine

To a solution of 2,4-dichloro-5-(trifluoromethyl)pyrimidine (91.0 mg, 0.42 mmol), (3R,4S)-1-(1H-imidazol-4-ylsulfonyl)-3-methylpiperidin-4-amine (103 mg, 0.42 mmol) in DCE (200 mL) tert-butanol (200 mL) was added Zinc chloride (85.7 mg, 0.63 mmol) at 0° C. The mixture was stirred for 1 h, followed by the dropwise addition of triethylamine (63.7 mg, 0.63 mmol) at 0° C. The mixture was then stirred at 40° C. overnight. LC-MS showed that the starting material was consumed. The solvent was removed under reduced pressure. The mixture was then added H₂O (10.0 mL) and extracted with ethyl acetate (10.0 mL×3). The combined organic layers were dried over Na₂SO₄, filtered, concentrated. The crude was purified by Prep-HPLC on C₁₈ column (30×250 mm, 10 m) using mobile phase 15 to 70% MeCN/H₂O (tR=20 min) to afford 4-chloro-N-[(3R,4S)-1-(1H-imidazol-4-ylsulfonyl)-3-methylpiperidin-4-yl]-5-(trifluoromethyl) pyrimidin-2-amine (14 mg, 7.85% yield). LCMS calc. for C₁₄H₁₇ClF₃N₆O₂S [M+H]⁺: m/z=425.08; found 424.8.

### Example 1. 2-Methyl-5-[2-[[1-(1-methylimidazol-4-yl)sulfonylpiperidin-4-yl]amino]-5-(trifluoromethyl)pyrimidin-4-yl]thiophene-3-carbonitrile

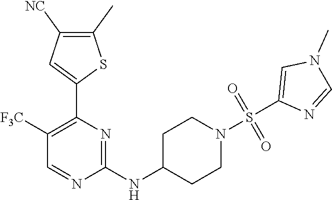 [SMILES: Cc1sc(-c2nc(NC3CCN(S(=O)(=O)c4cn(C)cn4)CC3)ncc2C(F)(F)F)cc1C#N]

### Step 1. Tert-butyl 4-[[4-(4-cyano-5-methylthiophen-2-yl)-5-(trifluoromethyl)pyrimidin-2-yl]amino]piperidine-1-carboxylate

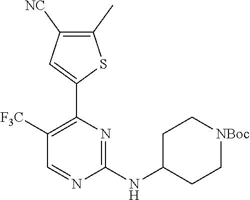 [SMILES: Cc1sc(-c2nc(NC3CCN(C(=O)OC(C)(C)C)CC3)ncc2C(F)(F)F)cc1C#N]

A mixture of 5-bromo-2-methylthiophene-3-carbonitrile (Intermediate 14) (80.0 mg, 0.40 mmol), Pd(PPh₃)₄ (91.5 mg, 0.08 mmol), tert-butyl 4-[[4-chloro-5-(trifluoromethyl)pyrimidin-2-yl]amino]piperidine-1-carboxylate (Intermediate 20) (165.8 mg, 0.44 mmol), hexamethylditin (0.16 mL, 0.79 mmol) in 1,4-dioxane (20 mL) was sparging with N₂ for 5 min. The mixture was then stirred at 100° C. in a sealed tube for 12 h. LCMS showed that the starting material was consumed. The reaction was then cooled to RT and KF (5 mL, 10% aq.) was added. The mixture was stirred at RT for 1 h. The solvent was then removed under reduced pressure. The residue was extracted with ethyl acetate (10 mL×2). The combined organic layers were dried over Na₂SO₄, filtered, and concentrated. The crude was purified by prep-HPLC on C₁₈ column (10-70% MeCN in 0.1% TFA(aq), pH=2) to afford the title compound (160.1 mg, 85.7% yield). LCMS calc. for C₂₁H₂₅F₃N₅O₂S [M+H]⁺: m/z=468.2; Found 468.0.

### Step 2. 2-Methyl-5-[2-(piperidin-4-ylamino)-5-(trifluoromethyl)pyrimidin-4-yl]thiophene-3-carbonitrile

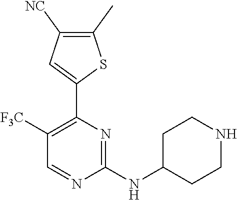 [SMILES: Cc1sc(-c2nc(NC3CCNCC3)ncc2C(F)(F)F)cc1C#N]

To a solution of tert-butyl 4-[[4-(4-cyano-5-methylthiophen-2-yl)-5-(trifluoromethyl)-pyrimidin-2-yl]amino]piperidine-1-carboxylate (160.0 mg, 0.34 mmol) in DCM (4 mL) was added TFA (2 mL). The mixture was stirred at RT for 1 h. LCMS showed that the starting material was consumed. The reaction mixture was concentrated under reduced pressure to afford the title compound (125.0 mg, quantitative yield) which was directly used in the next step without further purification. LCMS calc. for C₁₆H₁₇F₃N₅S [M+H]⁺: m/z=368.1; Found 368.2.

### Step 3. 2-Methyl-5-[2-[[1-(1-methylimidazol-4-yl)sulfonylpiperidin-4-yl]amino]-5-(trifluoromethyl)pyrimidin-4-yl]thiophene-3-carbonitrile

To a solution of 2-methyl-5-[2-(piperidin-4-ylamino)-5-(trifluoromethyl)pyrimidin-4-yl]thiophene-3-carbonitrile (23.0 mg, 0.06 mmol) in DMA (1 mL) was added 1-methyl-1H-imidazole-4-sulfonyl chloride (12.4 mg, 0.07 mmol) at 0° C., followed by the addition of DIPEA (80.9 mg, 0.63 mmol). The mixture was then stirred at RT for 3 h. LCMS showed the starting material was consumed. The crude was purified by prep-HPLC on C₁₈ column (10-60% MeCN in 0.1% TFA(aq), pH=2) to afford the title compound (21.2 mg, 64.8% yield). LCMS calc. for C₂₀H₂₁F₃N₇O₂S₂ [M+H]⁺: m/z=512.1; Found 512.0. ¹H NMR (300 MHz, DMSO) δ 8.65 (d, J=4.9 Hz, 1H), 8.24 (dd, J=23.3, 7.3 Hz, 1H), 7.89-7.80 (m, 2H), 7.72 (d, J=8.7 Hz, 1H), 3.74 (s, 1H), 3.73 (s, 3H), 3.59 (d, J=12.5 Hz, 2H), 2.77-2.57 (m, 5H), 1.94 (t, J=15.0 Hz, 2H), 1.68-1.51 (m, 2H).

### Example 2. N-(1-((1-methyl-1H-pyrazol-4-yl)sulfonyl)piperidin-4-yl)-4-(5-methylthiazol-2-yl)-5-(trifluoromethyl)pyrimidin-2-amine

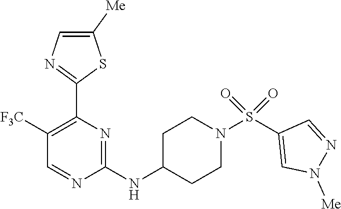 [SMILES: Cc1cnc(-c2nc(NC3CCN(S(=O)(=O)c4cnn(C)c4)CC3)ncc2C(F)(F)F)s1]

### Step 1. Tert-butyl 4-((4-(5-methylthiazol-2-yl)-5-(trifluoromethyl)pyrimidin-2-yl)amino) piperidine-1-carboxylate

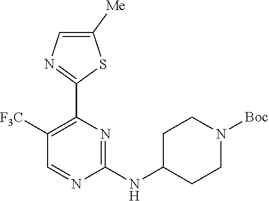 [SMILES: Cc1cnc(-c2nc(NC3CCN(C(=O)OC(C)(C)C)CC3)ncc2C(F)(F)F)s1]

To a tube was added tert-butyl 4-((4-chloro-5-(trifluoromethyl)pyrimidin-2-yl)amino) piperidine-1-carboxylate (Intermediate 20) (100 mg, 0.262 mmol), 5-methyl-2-(trimethylstannyl) thiazole (132 mg, 0.341 mmol), tetrakis(triphenylphosphine)palladium(0) (45.0 mg, 0.040 mmol) and dioxane (2.60 mL). The tube was sealed, and the reaction mixture was degassed by sparging with N₂. The reaction mixture was stirred at 90° C. for 3 h and then cooled to rt. The reaction mixture was concentrated to dryness and redissolved in acetonitrile and centrifuged off any undissolved solids. The clear solution was then purified by prep-HPLC on a C₁₈ column (20-80% MeCN in 0.1% TFA(aq), pH=2). The fractions of sufficient purity were basified with saturated NaHCO₃ (aq) and collected to afford the title compound (62.0 mg, 0.140 mmol, 53.0% yield). LCMS calc. for C₁₉H₂₅F₃N₅O₂S [M+H]⁺: m/z=444.2; found 444.0.

### Step 2. 4-(5-Methylthiazol-2-yl)-N-(piperidin-4-yl)-5-(trifluoromethyl)pyrimidin-2-amine dihydrochloride

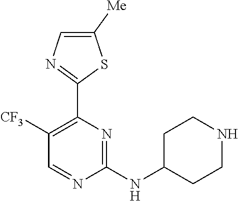 [SMILES: Cc1cnc(-c2nc(NC3CCNCC3)ncc2C(F)(F)F)s1]

To a vial containing tert-butyl 4-((4-(5-methylthiazol-2-yl)-5-(trifluoromethyl)pyrimidin-2-yl)amino)piperidine-1-carboxylate (62.0 mg, 0.140 mmol) was added HCl (2.0 M in isopropyl acetate, 1.40 mL). The reaction mixture was stirred at rt until full consumption of the starting material. The reaction mixture was then concentrated to dryness to afford the title compound as a crude solid (HCl salt) which was used in the next step without further purification. LCMS calc. for C₁₄H₁₇F₃N₅S [M+H]⁺: m/z=344.1; found 344.1.

### Step 3. N-(1-((1-methyl-1H-pyrazol-4-yl)sulfonyl)piperidin-4-yl)-4-(5-methylthiazol-2-yl)-5-(trifluoromethyl)pyrimidin-2-amine

4-(5-methylthiazol-2-yl)-N-(piperidin-4-yl)-5-(trifluoromethyl)pyrimidin-2-amine dihydrochloride (28.3 mg, 0.068 mmol) and N,N-diisopropylethylamine (60.0 μL, 0.340 mmol) were dissolved in anhydrous DMAc (400 μL) and cooled to 0° C. Next a solution of 1-methyl-1H-pyrazole-4-sulfonyl chloride (25 mg, 0.136 mmol) in DMAc (280 μL) was added dropwise to the reaction and the resulting mixture was stirred for 10 minutes at 0° C. The reaction mixture was then diluted with MeOH (2.00 mL) and purified directly by prep-HPLC on a C₁₈ column (15-70% MeCN in 0.1% TFA(aq), pH=2) to afford the title compound (31.0 mg, 0.064 mmol, 94.0% yield) as TFA salt. ¹H NMR (300 MHz, DMSO-d6) δ 8.68 (s, 1H), 8.35 (d, J=7.3 Hz, 1H), 8.24 (dd, J=21.6, 7.4 Hz, 1H), 7.85-7.77 (m, 2H), 3.92 (d, J=3.0 Hz, 3H), 3.85-3.70 (m, 1H), 3.55-3.45 (m, 2H), 2.57-2.53 (m, 3H), 2.51-2.38 (m, 2H), 2.11-1.90 (m, 2H), 1.75-1.55 (m, 2H). LCMS calc. for C₁₈H₂₁F₃N₇O₂S₂ [M+H]⁺: m/z=488.1; found 488.0.

### Example 3. 2-Methyl-1-(2-(2-((1-((1-methyl-1H-imidazol-4-yl)sulfonyl)piperidin-4-yl)amino)-5-(trifluoromethyl)pyrimidin-4-yl)thiazol-5-yl)propan-2-ol

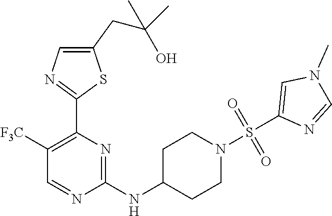 [SMILES: Cn1cnc(S(=O)(=O)N2CCC(Nc3ncc(C(F)(F)F)c(-c4ncc(CC(C)(C)O)s4)n3)CC2)c1]

### Step 1. Tert-butyl 4-((4-(5-(2-hydroxy-2-methylpropyl)thiazol-2-yl)-5-(trifluoromethyl)-pyrimidin-2-yl)amino)piperidine-1-carboxylate

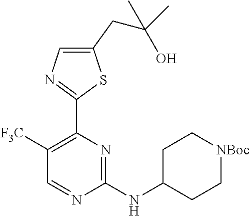 [SMILES: CC(C)(O)Cc1cnc(-c2nc(NC3CCN(C(=O)OC(C)(C)C)CC3)ncc2C(F)(F)F)s1]

To a solution of 1-(2-bromo-1,3-thiazol-5-yl)-2-methylpropan-2-ol (Intermediate 7) (27.8 mg, 0.118 mmol) and tert-butyl 4-[[5-(trifluoromethyl)-4-trimethylstannylpyrimidin-2-yl]amino]piperidine-1-carboxylate (Intermediate 23) (60.0 mg, 0.118 mmol) in 1,4-dioxane (1.00 mL) was added bis(triphenylphosphine)palladium(II) dichloride (16.5 mg, 0.0236 mmol), copper(I) iodide (4.5 mg, 0.024 mmol) and lithium chloride (15.0 mg, 0.354 mmol). The reaction mixture was bubbled with N₂ for 1 min and stirred at 100° C. for 3 h. LCMS showed the starting material was consumed. The reaction was quenched with KF(aq) solution and stirred at RT for 1 h. The layer was separated, and the aqueous layer was extracted with DCM (2.00 mL×3). The combined organic layers were dried over Na₂SO₄, filtered and concentrated. The residue was purified by prep-HPLC on a C₁₈ column eluting with MeCN/H₂O (20-70%) with 0.1% TFA to yield the title compound (27.1 mg, 45.7% yield). LCMS calculated for C₂₂H₃₁F₃N₅O₃S (M+H)⁺: m/z=502.21; Found 502.1.

### Step 2. 2-Methyl-1-(2-(2-(piperidin-4-ylamino)-5-(trifluoromethyl)pyrimidin-4-yl)thiazol-5-yl)propan-2-ol

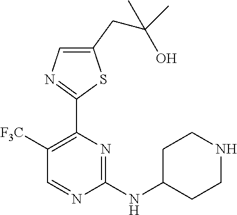 [SMILES: CC(C)(O)Cc1cnc(-c2nc(NC3CCNCC3)ncc2C(F)(F)F)s1]

To a solution of tert-butyl 4-((4-(5-(2-hydroxy-2-methylpropyl)thiazol-2-yl)-5-(trifluoro-methyl)pyrimidin-2-yl)amino)piperidine-1-carboxylate (27.1 mg, 0.0538 mmol) in DCM (0.50 mL) was added 2,2,2-trifluoroacetic acid (0.20 mL). The reaction mixture was stirred at RT for 1 h. LCMS showed the starting material was consumed. The solvent was removed under reduced pressure to yield the title compound (22.0 mg, quant.), which was used for the next reaction without further purification. LCMS calculated for C₁₇H₂₃F₃N₅OS (M+H)⁺: m/z=402.16; Found 402.1.

### Step 3. 2-Methyl-1-(2-(2-((1-((1-methyl-1H-imidazol-4-yl)sulfonyl)piperidin-4-yl)amino)-5-(trifluoromethyl)pyrimidin-4-yl)thiazol-5-yl)propan-2-ol

To a solution of 2-methyl-1-(2-(2-(piperidin-4-ylamino)-5-(trifluoromethyl)pyrimidin-4-yl)thiazol-5-yl)propan-2-ol (11.0 mg, 0.0274 mmol) and N,N-diisopropylethylamine (14.3 μL, 0.0822 mmol) in DMA (1.00 mL) was added 1-methylimidazole-4-sulfonyl chloride (5.9 mg, 0.0330 mmol). The reaction mixture was stirred at RT for 20 min. LCMS showed the starting material was consumed. The reaction was diluted with MeOH (2.00 mL) and purified by prep-HPLC on a C₁₈ column eluting with MeCN/H₂O (20-60%) with 0.1% TFA to yield the title compound (4.7 mg, 30.4% yield). ¹H NMR (300 MHz, DMSO) δ 8.69 (s, 1H), 8.27-8.18 (m, 1H), 7.83 (dd, J=10.5, 4.4 Hz, 3H), 3.78 (s, 1H), 3.73 (s, 3H), 3.59 (d, J=12.2 Hz, 2H), 2.96 (s, 2H), 2.67 (dd, J=29.1, 10.7 Hz, 2H), 1.96 (dd, J=21.5, 10.6 Hz, 2H), 1.60 (d, J=11.7 Hz, 2H), 1.12 (s, 6H). LCMS calculated for C₂₁H₂₇F₃N₇O₃S₂ (M+H)⁺: m/z=546.16; Found 546.1.

### Examples 4-68 and 85

Examples 4-68 and 85 shown below in Table 1 were prepared in accordance with the synthetic protocols set forth in Example 1, 2, or 3 above using the appropriate intermediates, as well as commercial starting materials.

TABLE 1
Examples 4-68 and 85

| Ex. | Molecular Structure and Name | Analytical Data (LCMS and/or NMR) |
|---|---|---|
| 4 | 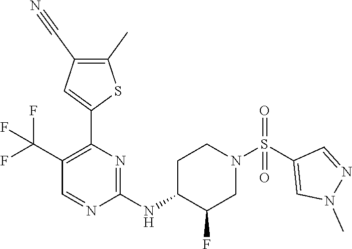 [SMILES: Cc1sc(-c2nc(N[C@@H]3CCN(S(=O)(=O)c4cnn(C)c4)C[C@H]3F)ncc2C(F)(F)F)cc1C#N] | LCMS calc. for C₂₀H₂₀F₄N₇O₂S₂ [M + H]⁺: m/z = 530.1; Found 530.1. ¹H NMR (300 MHz, DMSO) δ 8.68 (s, 1H), 8.51-8.34 (m, 2H), 7.84 (d, J = 3.1 Hz, 1H), 7.73 (d, J = 7.8 Hz, 1H), 4.87- 4.61 (m, 1H), 4.17-4.05 (m, 1H), 3.92 (s, 3H), 3.72-3.47 (m, 1H), 3.46-3.24 (m, 1H), 2.96-2.70 (m, 2H), 2.67 (s, 3H), 2.09- 1.95 (m, 1H), 1.75-1.58 (m, 1H). |
| | 5-(2-(((3R,4R)-3-fluoro-1-((1-methyl- | |
| | 1H-pyrazol-4-yl)sulfonyl)piperidin-4- | |
| | yl)amino)-5-(trifluoromethyl)pyrimidin- | |
| | 4-yl)-2-methylthiophene-3-carbonitrile | |
| 5 | 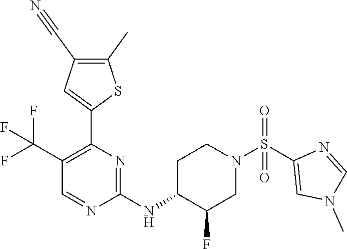 [SMILES: Cc1sc(-c2nc(N[C@@H]3CCN(S(=O)(=O)c4cn(C)cn4)C[C@H]3F)ncc2C(F)(F)F)cc1C#N] | LCMS calc. for C₂₀H₂₀F₄N₇O₂S₂ [M + H]⁺: m/z = 530.1; Found 530.1 ¹H NMR (300 MHz, DMSO) δ 8.68 (s, 1H), 8.41 (dd, J = 25.3, 7.8 Hz, 1H), 7.88 (s, 2H), 7.73 (d, J = 7.5 Hz, 1H), 4.82- 4.54 (m, 1H), 4.25-4.00 (m, 1H), 3.80-3.70 (m, 1 H), 3.72 (s, 3H), 3.52-3.44 (m, 1H), 3.04-2.75 (m, 2H), 2.68 (s, 3H), 2.08- 1.93 (m, 1H), 1.73-1.56 (m, 1H). |
| | 5-(2-(((3R,4R)-3-fluoro-1-((1-methyl- | |
| | 1H-imidazol-4-yl)sulfonyl)piperidin-4- | |
| | yl)amino)-5-(trifluoromethyl)pyrimidin- | |
| | 4-yl)-2-methylthiophene-3-carbonitrile | |
| 6 | 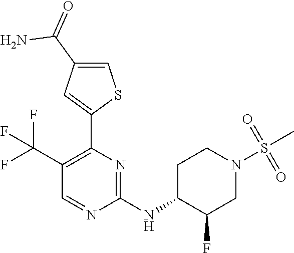 [SMILES: CS(=O)(=O)N1CC[C@@H](Nc2ncc(C(F)(F)F)c(-c3cc(C(N)=O)cs3)n2)[C@H](F)C1] | LCMS calc. for C₁₆H₁₈F₄N₅O₃S₂ [M + H]⁺: m/z = 468.1; Found 468.1. ¹H NMR (300 MHz, DMSO) δ 8.71 (d, J = 6.5 Hz, 1H), 8.50-8.28 (m, 2H), 8.08- 7.89 (m, 2H), 7.38 (s, 1H), 4.82-4.50 (m, 1H), 4.04-3.89 (m, 1H), 3.83-3.70 (m, 1H), 3.54-3.46 (m, 1H), 3.22-3.02 (m, 2H), 2.96 (s, 3H), 2.18-1.98 (m, 1H), 1.75- 1.55 (m, 1H). |
| | 5-(2-(((3R,4R)-3-fluoro-1-(methyl- | |
| | sulfonyl)piperidin-4-yl)amino)-5- | |
| | (trifluoromethyl)pyrimidin-4- | |
| | yl)thiophene-3-carboxamide | |
| 7 | 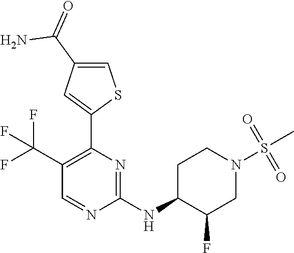 [SMILES: CS(=O)(=O)N1CC[C@H](Nc2ncc(C(F)(F)F)c(-c3cc(C(N)=O)cs3)n2)[C@H](F)C1] | LCMS calc. for C₁₆H₁₈F₄N₅O₃S₂ [M + H]⁺: m/z = 468.1; Found 468.0. ¹H NMR (300 MHz, DMSO) δ 8.70 (s, 1H), 8.43 (d, J = 4.9 Hz, 1H), 8.42-8.31 (m, 1H), 8.06-7.92 (m, 2H), 7.39 (s, 1H), 5.17-4.80 (m, 1H), 4.25-4.00 (m, 1H), 3.98-3.76 (m, 2H), 3.34-2.96 (m, 2H), 2.93 (d, J = 6.3 Hz, 3H), 2.05-1.94 (m, 1H), 1.90-1.7x6 (m, 1H). |
| | 5-(2-(((3R,4S)-3-fluoro-1-(methyl- | |
| | sulfonyl)piperidin-4-yl)amino)-5- | |
| | (trifluoromethyl)pyrimidin-4- | |
| | yl)thiophene-3-carboxamide | |
| 8 | 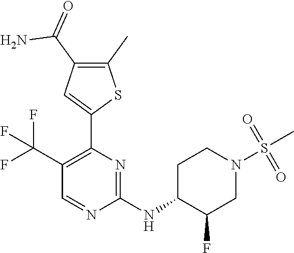 [SMILES: Cc1sc(-c2nc(N[C@@H]3CCN(S(C)(=O)=O)C[C@H]3F)ncc2C(F)(F)F)cc1C(N)=O] | LCMS calc. for C₁₇H₂₀F₄N₅O₃S₂ [M + H]⁺: m/z = 482.1; Found 482.1. ¹H NMR (300 MHz, DMSO) δ 8.67 (d, J = 7.2 Hz, 1H), 8.32 (dd, J = 24.9, 8.2 Hz, 1H), 7.78 (s, 1H), 7.70 (s, 1H), 7.36 (s, 1H), 4.75-4.55 (m, 1H), 4.31-4.10 (m, 1H), 3.82-3.68 (m, 1H), 3.54-3.46 (m, 1H), 3.23-3.00 (m, 2H), 2.96 (t, J = 4.0 Hz, 3H), 2.66 (s, 3H), 2.15-1.95 (m, 1H), 1.70- 1.55 (m, 1H). |
| | 5-(2-(((3R,4R)-3-fluoro-1-(methyl- | |
| | sulfonyl)piperidin-4-yl)amino)-5- | |
| | (trifluoromethyl)pyrimidin-4-yl)-2- | |
| | methylthiophene-3-carboxamide | |
| 9 | 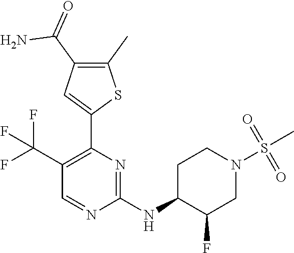 [SMILES: Cc1sc(-c2nc(N[C@H]3CCN(S(C)(=O)=O)C[C@H]3F)ncc2C(F)(F)F)cc1C(N)=O] | LCMS calc. for C₁₇H₂₀F₄N₅O₃S₂ [M + H]⁺: m/z = 482.1; Found 482.0. ¹H NMR (300 MHz, DMSO) δ 8.67 (s, 1H), 8.30 (dd, J = 22.7, 7.2 Hz, 1H), 7.79 (s, 1H), 7.70 (s, 1H), 7.36 (s, 1H), 5.08- 4.85 (m, 1H), 3.94-3.80 (m, 2H), 3.75- 3.61 (m, 1H), 3.31-3.00 (m, 2H), 2.93 (d, J = 7.8 Hz, 3H), 2.66 (s, 3H), 2.04-1.88 (m, 1H), 1.86-1.72 (m, 1H). |
| | 5-(2-(((3R,4S)-3-fluoro-1-(methyl- | |
| | sulfonyl)piperidin-4-yl)amino)-5- | |
| | (trifluoromethyl)pyrimidin-4-yl)-2- | |
| | methylthiophene-3-carboxamide | |
| 10 | 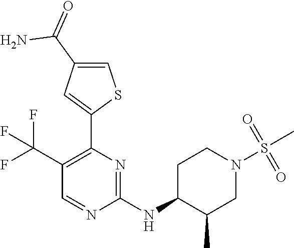 [SMILES: C[C@@H]1CN(S(C)(=O)=O)CC[C@@H]1Nc1ncc(C(F)(F)F)c(-c2cc(C(N)=O)cs2)n1] | LCMS calc. for C₁₇H₂₁F₃N₅O₃S₂ [M + H]⁺: m/z = 464.1; Found 464.0. ¹H NMR (300 MHz, DMSO) δ 8.68 (d, J = 9.6 Hz, 1H), 8.42 (d, J = 8.7 Hz, 1H), 8.24 (dd, J = 29.2, 8.1 Hz, 1H), 8.00 (d, J = 15.1 Hz, 2H), 7.38 (s, 1H), 4.24-4.16 (m, 1H), 3.39-3.02 (m, 4H), 2.86 (d, J = 5.4 Hz, 3H), 2.29-2.14 (m, 1H), 1.90-1.71 (m, 2H), 0.89 (t, J = 7.0 Hz, 3H). |
| | 5-(2-(((3R,4S)-3-methyl-1-(methyl- | |
| | sulfonyl)piperidin-4-yl)amino)-5- | |
| | (trifluoromethyl)pyrimidin-4- | |
| | yl)thiophene-3-carboxamide | |
| 11 | 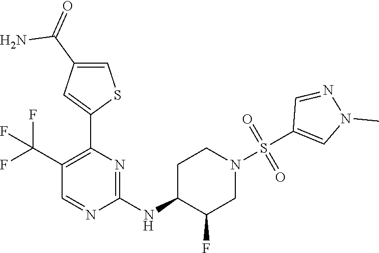 | LCMS calc. for C₁₉H₂₀F₄N₇O₃S₂ [M + H]⁺: m/z = 534.1; Found 534.1. ¹H NMR (300 MHz, DMSO) δ 8.67 (d, J = 10.0 Hz, 1H), 8.41 (s, 1H), 8.33 (dd, J = 14.0, 7.9 Hz, 2H), 7.98 (d, J = 13.5 Hz, 2H), 7.81 (d, J = 9.9 Hz, 1H), 7.38 (s, 1H), 5.15-4.80 (m, 1H), 4.23-3.95 (m, 1H), 3.92 (d, J = 2.5 Hz, 3H), 3.90-3.75 (m, 1H), 3.70-3.59 (m, 1H), 2.82-2.53 (m, 2H), 2.08-1.88 (m, 1H), 1.88-1.70 (m, 1H). |
| | 5-(2-(((3R,4S)-3-fluoro-1-((1-methyl-1H- | |
| | pyrazol-4-yl)sulfonyl)piperidin-4- | |
| | yl)amino)-5-(trifluoromethyl)pyrimidin- | |
| | 4-yl)thiophene-3-carboxamide | |
| 12 | 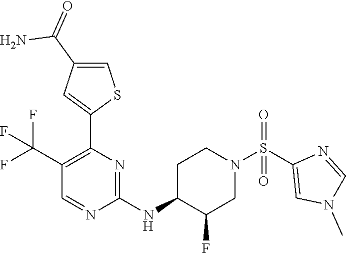 | LCMS calc. for C₁₉H₂₀F₄N₇O₃S₂ [M + H]⁺: m/z = 534.1; Found 534.1. ¹H NMR (300 MHz, DMSO) δ 8.67 (d, J = 6.8 Hz, 1H), 8.42 (d, J = 1.0 Hz, 1H), 8.33 (dd, J = 18.3, 7.2 Hz, 1H), 8.05-7.91 (m, 2H), 7.85 (t, J = 3.9 Hz, 2H), 7.38 (s, 1H), 5.15-4.80 (m, 1H), 3.94-3.83 (m, 2H), 3.73 (s, 3H), 3.70-3.62 (m, 1H), 2.99- 2.78 (m, 1H), 2.75-2.61 (m, 1H), 2.09- 1.87 (m, 1H), 1.87-1.71 (m, 1H). |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)thiophene-3-carboxamide | |
| 13 | 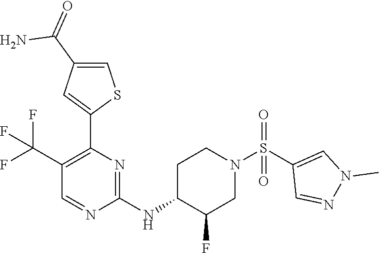 | LCMS calc. for C₁₉H₂₀F₄N₇O₃S₂ [M + H]⁺: m/z = 534.1; Found 534.1. ¹H NMR (300 MHz, DMSO) δ 8.67 (s, 1H), 8.46-8.27 (m, 3H), 8.05-7.90 (m, 2H), 7.84 (s, 1H), 7.37 (s, 1H), 4.83-4.60 (m, 1H), 3.94 (s, 3H), 3.67-3.29 (m, 3H), 2.91-2.64 (m, 2H), 2.07-1.93 (m, 1H), 1.76-1.60 (m, 1H). |
| | 5-(2-(((3R,4R)-3-fluoro-1-((1-methyl- | |
| | 1H-pyrazol-4-yl)sulfonyl)piperidin-4- | |
| | yl)amino)-5-(trifluoromethyl)pyrimidin- | |
| | 4-yl)thiophene-3-carboxamide | |
| 14 | 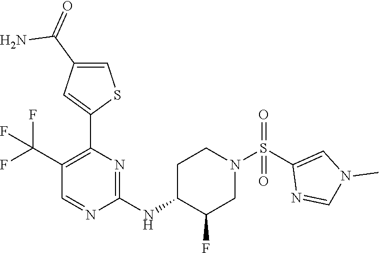 | LCMS calc. for C₁₉H₂₀F₄N₇O₃S₂ [M + H]⁺: m/z = 534.1; Found 534.1. ¹H NMR (300 MHz, DMSO) δ 8.67 (s, 1H), 8.45-8.29 (m, 2H), 8.04-7.92 (m, 2H), 7.88 (s, 2H), 7.37 (s, 1H), 4.84-4.55 (m, 1H), 4.24-4.01 (m, 1H), 3.73 (s, 3H), 3.55-3.46 (m, 2H), 2.99-2.72 (m, 2H), 2.15-1.92 (m, 1H), 1.74-1.54 (m, 1H). |
| | 5-(2-(((3R,4R)-3-fluoro-1-((1-methyl- | |
| | 1H-imidazol-4-yl)sulfonyl)piperidin-4- | |
| | yl)amino)-5-(trifluoromethyl)pyrimidin- | |
| | 4-yl)thiophene-3-carboxamide | |
| 15 | 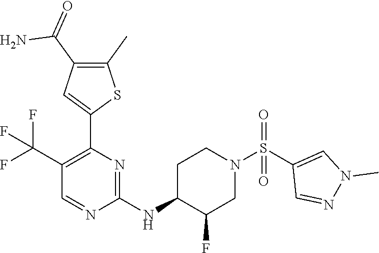 | LCMS calc. for C₂₀H₂₂F₄N₇O₃S₂ [M + H]⁺: m/z = 548.1; Found 548.1. ¹H NMR (300 MHz, DMSO) δ 8.63 (d, J = 9.2 Hz, 1H), 8.36 (d, J = 9.1 Hz, 1H), 8.27 (dd, J = 18.4, 7.0 Hz, 1H), 7.87-7.74 (m, 2H), 7.69 (s, 1H), 7.35 (s, 1H), 4.96 (dd, J = 49.2, 19.2 Hz, 1H), 4.20-3.96 (m, 1H), 3.92 (s, 3H), 3.88-3.69 (m, 2H), 2.87-2.69 (m, 1H), 2.64 (d, J = 3.4 Hz, 3H), 2.58-2.50 (m, 1H), 2.04-1.82 (m, 1H), 1.82-1.70 (m, 1H). |
| | 5-(2-(((3R,4S)-3-fluoro-1-((1-methyl-1H- | |
| | pyrazol-4-yl)sulfonyl)piperidin-4- | |
| | yl)amino)-5-(trifluoromethyl)pyrimidin- | |
| | 4-yl)-2-methylthiophene-3-carboxamide | |
| 16 | 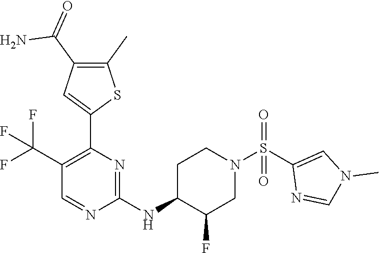 | LCMS calc. for C₂₀H₂₂F₄N₇O₃S₂ [M + H]⁺: m/z = 548.1; Found 548.0. ¹H NMR (300 MHz, DMSO) δ 8.64 (d, J = 6.0 Hz, 1H), 8.26 (dd, J = 21.1, 7.3 Hz, 1H), 7.91-7.82 (m, 2H), 7.77 (s, 1H), 7.68 (s, 1H), 7.35 (s, 1H), 5.08-4.75 (m, 1H), 4.03-3.83 (m, 2H), 3.73 (s, 3H), 3.69-3.60 (m, 1H), 3.02-2.67 (m, 2H), 2.64 (s, 3H), 2.05-1.92 (m, 1H), 1.82- 1.70 (m, 1H). |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)-2-methylthiophene-3-carboxamide | |
| 17 | 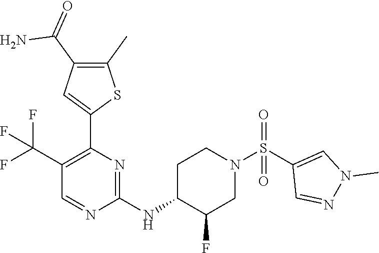 | LCMS calc. for C₂₀H₂₂F₄N₇O₃S₂ [M + H]⁺: m/z = 548.1; Found 548.1. ¹H NMR (300 MHz, DMSO) δ 8.63 (s, 1H), 8.39 (s, 1H), 8.29 (dd, J = 24.2, 7.8 Hz, 1H), 7.84 (s, 1H), 7.76 (s, 1H), 7.69 (s, 1H), 7.35 (s, 1H), 4.85-4.60 (m, 1H), 4.25- 4.04 (m, 1H), 3.92 (s, 3H), 3.69-3.25 (m, 2H), 2.99-2.67 (m, 2H), 2.65 (s, 3H), 2.20-1.96 (m, 1H), 1.80-1.56 (m, 1H). |
| | 5-(2-(((3R,4R)-3-fluoro-1-((1-methyl- | |
| | 1H-pyrazol-4-yl)sulfonyl)piperidin-4- | |
| | yl)amino)-5-(trifluoromethyl)pyrimidin- | |
| | 4-yl)-2-methylthiophene-3-carboxamide | |
| 18 | 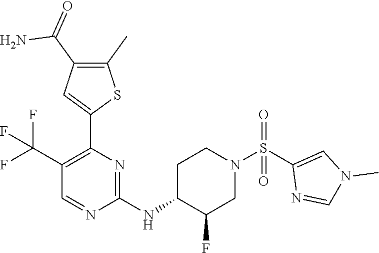 | LCMS calc. for C₂₀H₂₂F₄N₇O₃S₂ [M + H]⁺: m/z = 548.1; Found 548.1. ¹H NMR (300 MHz, DMSO) δ 8.63 (s, 1H), 8.29 (dd, J = 21.7, 7.7 Hz, 1H), 7.88 (s, 2H), 7.76 (s, 1H), 7.69 (s, 1H), 7.34 (s, 1H), 4.82-4.55 (m, 1H), 4.20-4.00 (m, 1H), 3.73 (s, 3H), 3.67-3.58 (m, 1H), 3.52- 3.42 (m, 1H), 3.01-2.76 (m, 2H), 2.65 (s, 3H), 2.04-1.94 (m, 1H), 1.75-1.55 (m, 1H). |
| | 5-(2-(((3R,4R)-3-fluoro-1-((1-methyl- | |
| | 1H-imidazol-4-yl)sulfonyl)piperidin-4- | |
| | yl)amino)-5-(trifluoromethyl)pyrimidin- | |
| | 4-yl)-2-methylthiophene-3-carboxamide | |
| 19 | 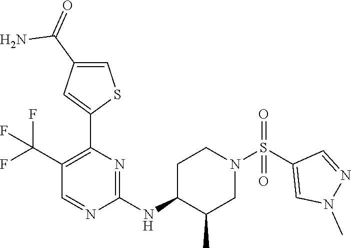 | LCMS calc. for C₂₀H₂₃F₃N₇O₃S₂ [M + H]⁺: m/z = 530.1; Found 530.1. ¹H NMR (300 MHz, DMSO) δ 8.64 (d, J = 7.5 Hz, 1H), 8.40 (d, J = 7.1 Hz, 1H), 8.33 (s, 1H), 8.22 (dd, J = 21.9, 7.7 Hz, 1H), 7.97 (d, J = 14.6 Hz, 2H), 7.78 (s, 1H), 7.37 (s, 1H), 4.08-4.03 (m, 1H), 3.92 (d, J = 3.1 Hz, 3H), 3.13-2.72 (m, 4H), 2.30-2.20 (m, 1H), 1.96-1.71 (m, 2H), 0.91 (d, J = 6.8 Hz, 3H). |
| | 5-(2-(((3R,4S)-3-methyl-1-((1-methyl- | |
| | 1H-pyrazol-4-yl)sulfonyl)piperidin-4- | |
| | yl)amino)-5-(trifluoromethyl)pyrimidin- | |
| | 4-yl)thiophene-3-carboxamide | |
| 20 | 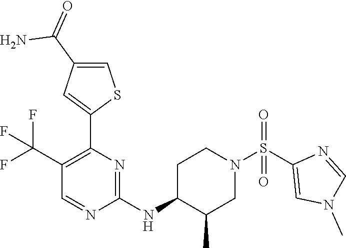 | LCMS calc. for C₂₀H₂₃F₃N₇O₃S₂ [M + H]⁺: m/z = 530.1; Found 530.0. ¹H NMR (300 MHz, DMSO) 8 8.64 (d, J = 6.7 Hz, 1H), 8.40 (d, J = 5.5 Hz, 1H), 8.19 (dd, J = 21.6, 7.6 Hz, 1H), 7.98 (d, J = 13.5 Hz, 2H), 7.83 (d, J = 13.7 Hz, 2H), 7.37 (s, 1H), 4.11-3.90 (m, 1H), 3.73 (s, 3H), 3.38-3.17 (m, 2H), 3.00-2.86 (m, 2H), 2.27-2.16 (m, 1H), 1.90-1.61 (m, 2H), 0.91 (d, J = 6.9 Hz, 3H). |
| | 5-(2-(((3R,4S)-3-methyl-1-((1-methyl- | |
| | 1H-imidazol-4-yl)sulfonyl)piperidin-4- | |
| | yl)amino)-5-(trifluoromethyl)pyrimidin- | |
| | 4-yl)thiophene-3-carboxamide | |
| 21 | 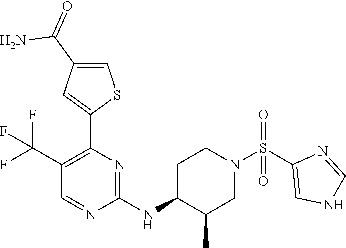 | LCMS calc. for C₁₉H₂₁F₃N₇O₃S₂ [M + H]⁺: m/z = 516.1; Found 516.1. ¹H NMR (300 MHz, DMSO) δ 8.64 (d, J = 7.5 Hz, 1H), 8.40 (d, J = 4.4 Hz, 1H), 8.20 (dd, J = 21.6, 7.7 Hz, 1H), 7.98 (d, J = 13.1 Hz, 3H), 7.80 (s, 1H), 7.37 (s, 1H), 4.10-3.94 (m, 1H), 3.39-3.17 (m, 2H), 3.00-2.82 (m, 2H), 2.26-2.10 (m, 1H), 1.93- 1.67 (m, 2H), 0.91 (d, J = 6.8 Hz, 3H). |
| | 5-(2-(((3R,4S)-1-((1H-imidazol-4- | |
| | yl)sulfonyl)-3-methylpiperidin-4- | |
| | yl)amino)-5-(trifluoromethyl)pyrimidin- | |
| | 4-yl)thiophene-3-carboxamide | |
| 22 | 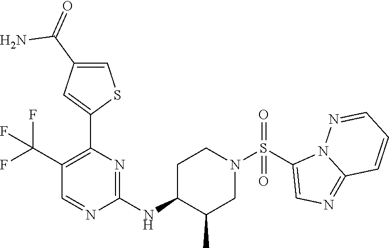 | LCMS calc. for C₂₂H₂₂F₃N₈O₃S₂ [M + H]⁺: m/z = 567.1; Found 567.1. ¹H NMR (300 MHz, DMSO) δ 8.78 (d, J = 4.5 Hz, 1H), 8.62 (d, J = 11.1 Hz, 1H), 8.43-8.33 (m, 2H), 8.24 (s, 1H), 8.13 (dd, J = 18.3, 8.3 Hz, 1H), 7.96 (d, J = 14.7 Hz, 2H), 7.54 (dd, J = 9.3, 4.5 Hz, 1H), 7.36 (s, 1H), 4.25-4.05 (m, 1H), 3.50- 3.18 (m, 4H), 2.25-2.06 (m, 1H), 1.90- 1.68 (m, 2H), 0.83 (d, J = 6.9 Hz, 3H). |
| | 5-(2-(((3R,4S)-1-(imidazo[1,2- | |
| | b]pyridazin-3-ylsulfonyl)-3-methyl- | |
| | piperidin-4-yl)amino)-5-(trifluoro- | |
| | methyl)pyrimidin-4-yl)thiophene-3- | |
| | carboxamide | |
| 23 | 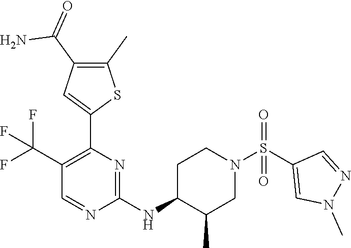 | LCMS calc. for C₂₁H₂₅F₃N₇O₃S₂ [M + H]⁺: m/z = 544.1; Found 544.1. ¹H NMR (300 MHz, DMSO) δ 8.60 (d, J = 8.5 Hz, 1H), 8.32 (s, 1H), 8.15 (dd, J = 28.1, 7.8 Hz, 1H), 7.80-7.72 (m, 2H), 7.68 (s, 1H), 7.34 (s, 1H), 4.10-4.02 (m, 1H), 3.92 (s, 3H), 3.21-3.01 (m, 1H), 2.99-2.90 (m, 2H), 2.90-2.70 (m, 1H), 2.64 (s, 3H), 2.27-2.18 (m, 1H), 1.90-1.65 (m, 2H), 0.97-0.83 (m, 3H). |
| | 2-methyl-5-(2-(((3R,4S)-3-methyl-1-((1- | |
| | methyl-1H-pyrazol-4-yl)sulfonyl) | |
| | piperidin-4-yl)amino)-5-(trifluoro- | |
| | methyl)pyrimidin-4-yl)thiophene-3- | |
| | carboxamide | |
| 24 | 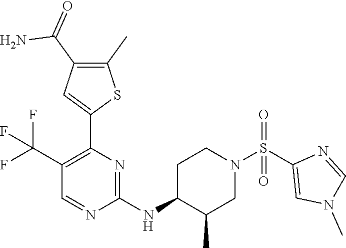 | LCMS calc. for C₂₁H₂₅F₃N₇O₃S₂ [M + H]⁺: m/z = 544.1; Found 544.2. ¹H NMR (300 MHz, DMSO) δ 8.61 (d, J = 7.4 Hz, 1H), 8.13 (dd, J = 26.1, 7.6 Hz, 1H), 7.87 (s, 1H), 7.81 (s, 1H), 7.75 (s, 1H), 7.68 (s, 1H), 7.35 (s, 1H), 4.09-3.91 (m, 1H), 3.73 (s, 3H), 3.38-3.12 (m, 2H), 3.05-2.83 (m, 2H), 2.64 (s, 3H), 2.27- 2.12 (m, 1H), 1.90-1.77 (m, 1H), 1.77-1.67 (m, 1H), 0.97-0.82 (m, 3H). |
| | 2-methyl-5-(2-(((3R,4S)-3-methyl-1-((1- | |
| | methyl-1H-imidazol-4-yl)sulfonyl) | |
| | piperidin-4-yl)amino)-5-(trifluoro- | |
| | methyl)pyrimidin-4-yl)thiophene-3- | |
| | carboxamide | |
| 25 | 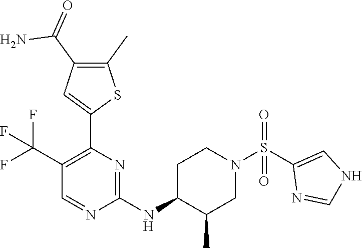 | LCMS calc. for C₂₀H₂₃F₃N₇O₃S₂ [M + H]⁺: m/z = 530.1; Found 530.1. ¹H NMR (300 MHz, DMSO) δ 8.60 (d, J = 8.9 Hz, 1H), 8.14 (dd, J = 26.3, 7.6 Hz, 1H), 7.92 (s, 1H), 7.77 (d, J = 9.7 Hz, 2H), 7.68 (s, 1H), 7.34 (s, 1H), 4.05-3.96 (m, 1H), 3.33-3.13 (m, 2H), 3.04-2.80 (m, 2H), 2.64 (s, 3H), 2.27-2.11 (m, 1H), 1.85- 1.58 (m, 2H), 0.89 (d, J = 6.7 Hz, 3H). |
| | 5-(2-(((3R,4S)-1-((1H-imidazol-4- | |
| | yl)sulfonyl)-3-methylpiperidin-4- | |
| | yl)amino)-5-(trifluoromethyl)pyrimidin- | |
| | 4-yl)-2-methylthiophene-3-carboxamide | |
| 26 | 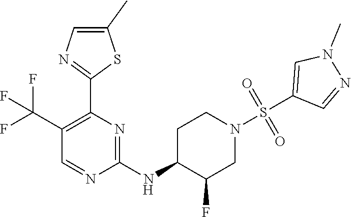 | LCMS calc. for C₁₈H₂₀F₄N₇O₂S₂ [M + H]⁺: m/z = 506.1; found 506.1 ¹H NMR (300 MHz, DMSO) δ 8.75-8.70 (m, 1H), 8.40-8.30 (m, 2H), 7.80-7.75 (m, 2H), 5.05-4.85 (m, 1H), 4.05-3.85 (m, 3H), 3.93 (s, 3H), 2.80-2.60 (m, 2H), 2.54 (s, 3H), 2.00-1.80 (m, 2H). |
| | N-((3R,4S)-3-fluoro-1-((1-methyl-1H- | |
| | pyrazol-4-yl)sulfonyl)piperidin-4-yl)-4- | |
| | (5-methylthiazol-2-yl)-5- | |
| | (trifluoromethyl)pyrimidin-2-amine | |
| 27 | 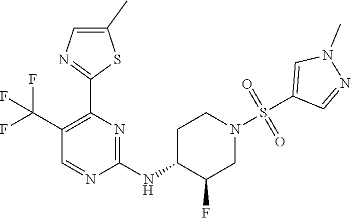 | LCMS calc. for C₁₈H₂₀F₄N₇O₂S₂ [M + H]⁺: m/z = 506.1; found 506.0. ¹H NMR (300 MHz, DMSO) δ 8.72 (s, 1H), 8.45-8.30 (m, 2H), 7.85-7.80 (m, 2H), 4.85-4.70 (m, 1H), 3.93 (s, 3H), 3.75-3.55 (m, 2H), 3.40-3.25 (m, 1H), 2.90-2.70 (m, 2H), 2.53 (s, 3H), 2.20-2.00 (m, 1H), 1.75- 1.70 (m, 1H). |
| | N-((3R,4R)-3-fluoro-1-((1-methyl-1H- | |
| | pyrazol-4-yl)sulfonyl)piperidin-4-yl)-4- | |
| | (5-methylthiazol-2-yl)-5- | |
| | (trifluoromethyl)pyrimidin-2-amine | |
| 28 | 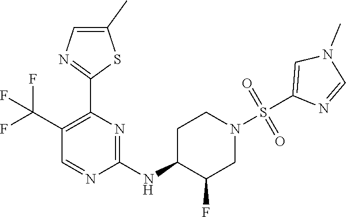 | LCMS calc. for C₁₈H₂₀F₄N₇O₂S₂ [M + H]⁺: m/z = 506.1; found 506.0. ¹H NMR (300 MHz, DMSO) δ 8.75-8.70 (m, 1H), 8.45-8.35 (m, 1H), 7.90-7.80 (m, 3H), 5.05-4.85 (m, 1H), 4.05-3.85 (m, 2H), 3.75-3.70 (m, 1H), 3.74 (s, 3H), 3.00-2.60 (m, 2H), 2.53 (s, 3H), 2.05-1.95 (m, 1H), 1.95-1.75 (m, 1H). |
| | N-((3R,4S)-3-fluoro-1-((1-methyl-1H- | |
| | imidazol-4-yl)sulfonyl)piperidin-4-yl)-4- | |
| | (5-methylthiazol-2-yl)-5-(trifluoro- | |
| | methyl)pyrimidin-2-amine | |
| 29 | 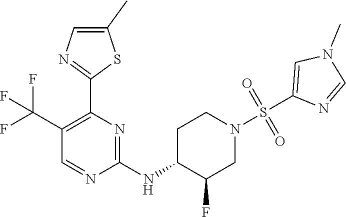 | LCMS calc. for C₁₈H₂₀F₄N₇O₂S₂ [M + H]⁺: m/z = 506.1; found 506.2. ¹H NMR (300 MHz, DMSO) δ 8.72 (s, 1H), 8.45-8.45 (m, 1H), 7.90-7.85 (m, 2H), 7.85-7.80 (m, 1H), 4.80-4.75 (m, 1H), 4.20-4.10 (m, 1H), 3.80-3.75 (m, 1H), 3.74 (s, 3H), 3.50-3.45 (m, 1H), 3.00-2.80 (m, 2H), 2.53 (s, 3H), 2.05-1.95 (m, 1H), 1.75- 1.70 (m, 1H). |
| | N-((3R,4R)-3-fluoro-1-((1-methyl-1H- | |
| | imidazol-4-yl)sulfonyl)piperidin-4-yl)-4- | |
| | (5-methylthiazol-2-yl)-5-(trifluoro- | |
| | methyl)pyrimidin-2-amine | |
| 30 | 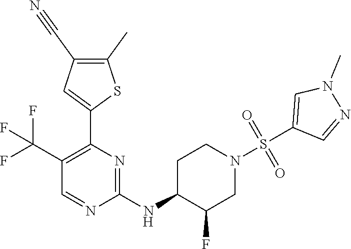 | LCMS calc. for C₂₀H₂₀F₄N₇O₂S₂ [M + H]⁺: m/z = 530.1; found 530.1. ¹H NMR (300 MHz, DMSO) δ 8.70-8.65 (m, 1H), 8.40-8.30 (m, 2H), 7.80-7.70 (m, 2H), 5.05-4.85 (m, 1H), 4.05-3.85 (m, 2H), 3.92 (s, 3H), 3.65-3.60 (m, 1H), 2.80-2.60 (m, 2H), 2.67 (s, 3H), 2.05-1.90 (m, 1H), 1.80-1.75 (m, 1H). |
| | 5-(2-(((3R,4S)-3-fluoro-1-((1-methyl-1H- | |
| | pyrazol-4-yl)sulfonyl)piperidin-4- | |
| | yl)amino)-5-(trifluoromethyl)pyrimidin- | |
| | 4-yl)-2-methylthiophene-3-carbonitrile | |
| 31 | 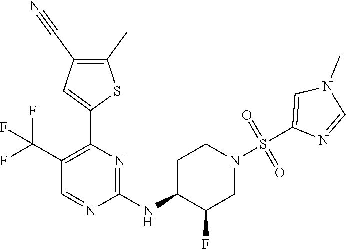 | LCMS calc. for C₂₀H₂₀F₄N₇O₂S₂ [M + H]⁺: m/z = 530.1; found 530.1. ¹H NMR (300 MHz, DMSO) δ 8.70-8.65 (m, 1H), 8.45-8.35 (m, 1H), 7.90-7.80 (m, 2H), 7.80-7.75 (m, 1H), 5.05-4.85 (m, 1H), 4.00-3.80 (m, 2H), 3.73 (s, 3H), 3.75-3.65 (m, 1H), 2.95-2.80 (m, 1H), 2.75-2.65 (m, 1H), 2.67 (s, 3H), 2.05-1.90 (m, 1H), 1.80- 1.70 (m, 1H). |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)-2-methylthiophene-3-carbonitrile | |
| 32 | 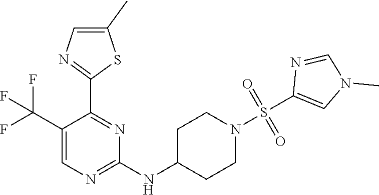 | LCMS calculated for C₁₈H₂₁F₃N₇O₂S₂ (M + H)⁺: m/z = 488.12; Found 488.2. ¹H NMR (300 MHz, CDCl₃) δ 8.61 (s, 1H), 7.72 (s, 1H), 7.53 (s, 1H), 7.47 (s, 1H), 5.45 (s, 1H), 3.96 (s, 1H), 3.79 (s, 5H), 2.99 (s, 2H), 2.56 (d, J = 1.1 Hz, 3H), 2.18 (d, J = 11.8 Hz, 2H), 0.86 (t, J = 7.8 Hz, 2H). |
| | N-(1-((1-methyl-1H-imidazol-4- | |
| | yl)sulfonyl)piperidin-4-yl)-4-(5- | |
| | methylthiazol-2-yl)-5- | |
| | (trifluoromethyl)pyrimidin-2-amine | |
| 33 | 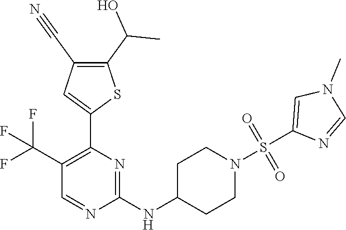 | LCMS calculated for C₂₁H₂₃F₃N₇O₃S₂ (M + H)⁺: m/z = 542.13; Found 542.1. ¹H NMR (300 MHz, DMSO-d₆) δ 8.66 (d, J = 3.8 Hz, 1H), 8.26 (dd, J = 20.6, 7.3 Hz, 1H), 7.88-7.80 (m, 2H), 7.74 (d, J = 10.3 Hz, 1H), 6.31 (bs, 1H), 5.17 (q, J = 6.3 Hz, 1H), 3.92-3.60 (m, 6H), 2.67 (dd, J = 25.4, 12.6 Hz, 2H), 2.11-1.84 (m, 2H), 1.72-1.53 (m, 2H), 1.50 (d, J = 6.4 Hz, 3H).34 |
| | 2-(1-hydroxyethyl)-5-(2-((1-((1-methyl- | |
| | 1H-imidazol-4-yl)sulfonyl)piperidin-4- | |
| | yl)amino)-5-(trifluoromethyl)pyrimidin- | |
| | 4-yl)thiophene-3-carbonitrile | |
| 34 | 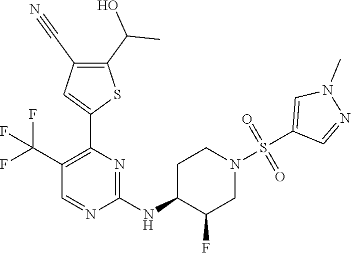 | LCMS calculated C₂₁H₂₂F₄N₇O₃S₂ (M + H)⁺: m/z = 560.12; Found 560.1. ¹H NMR (300 MHz, DMSO-i) δ 8.68 (d, J = 11.4 Hz, 1H), 8.42 (dd, J = 10.6, 7.5 Hz, 1H), 8.35 (d, J = 7.8 Hz, 1H), 7.80 (d, J = 6.4 Hz, 1H), 7.76 (d, J = 8.9 Hz, 1H), 6.31 (s, 1H), 5.25-5.13 (m, 1H), 4.94 (dd, J = 48.6, 14.5 Hz, 1H), 4.19- 3.96 (m, 1H), 3.91 (s, 3H), 3.83 (t, J = 11.8 Hz, 1H), 3.60 (d, J = 12.6 Hz, 1H), 2.71 (dd, J = 36.1, 12.9 Hz, 1H), 2.63-2.53 (m, 1H), 2.04-1.91 (m, 1H), 1.80 (t, J = 13.9 Hz, 1H), 1.50 (d, J = 6.4 Hz, 3H). |
| | 5-(2-(((3R,4S)-3-fluoro-1-((1-methyl-1H- | |
| | pyrazol-4-yl)sulfonyl)piperidin-4- | |
| | yl)amino)-5-(trifluoromethyl)pyrimidin- | |
| | 4-yl)-2-(1-hydroxyethyl)thiophene-3- | |
| | carbonitrile | |
| 35 | 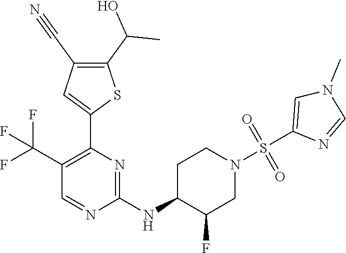 | LCMS calculated C₂₁H₂₂F₄N₇O₃S₂ (M + H)⁺: m/z = 560.12; Found 560.1. |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)-2-(1-hydroxyethyl)thiophene-3- | |
| | carbonitrile | |
| 36 | 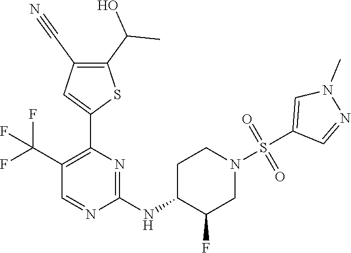 | LCMS calculated for C₂₁H₂₂F₄N₇O₃S₂ (M + H)⁺: m/z = 560.12; Found 560.1. ¹H NMR (300 MHz, DMSO-d₆) δ 8.69 (s, 1H), 8.53-8.34 (m, 2H), 7.83 (s, 1H), 7.75 (d, J = 9.0 Hz, 1H), 5.17 (q, J = 6.5 Hz, 1H), 4.93-4.55 (m, 1H), 4.29-4.08 (m, 1H), 3.92 (s, 3H), 3.57-3.22 (m, 2H), 3.06-2.63 (m, 2H), 2.18-1.95 (m, 1H), 1.78-1.60 (m, 1H), 1.50 (d, J = 6.4 Hz, 3H). |
| | 5-(2-(((3R,4R)-3-fluoro-1-((1-methyl- | |
| | 1H-pyrazol-4-yl)sulfonyl)piperidin-4- | |
| | yl)amino)-5-(trifluoromethyl)pyrimidin- | |
| | 4-yl)-2-(1-hydroxyethyl)thiophene-3- | |
| | carbonitrile | |
| 37 | 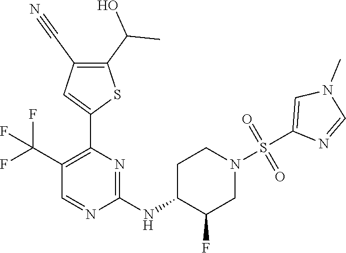 | LCMS calculated C₂₁H₂₂F₄N₇O₃S₂ (M + H)⁺: m/z = 560.12; Found 560.1. ¹H NMR (300 MHz, DMSO-d₆) δ 8.69 (s, 1H), 8.43 (dd, J = 19.9, 8.0 Hz, 1H), 7.87 (s, 2H), 7.75 (d, J = 9.2 Hz, 1H), 5.17 (q, J = 6.4 Hz, 1H), 4.91-4.45 (m, 1H), 4.17-4.04 (m, 1H), 3.81-3.75 (m, 1H), 3.73 (s, 3H), 3.70-3.61 (m, 1H), 3.54- 3.37 (m, 1H), 3.08-2.71 (m, 2H), 2.13- 1.95 (m, 1H), 1.71-1.57 (m, 1H), 1.50 (d, J = 6.4 Hz, 3H).3 |
| | 5-(2-(((3R,4R)-3-fluoro-1-((1-methyl- | |
| | 1H-imidazol-4-yl)sulfonyl)piperidin-4- | |
| | yl)amino)-5-(trifluoromethyl)pyrimidin- | |
| | 4-yl)-2-(1-hydroxyethyl)thiophene-3- | |
| | carbonitrile | |
| 38 | 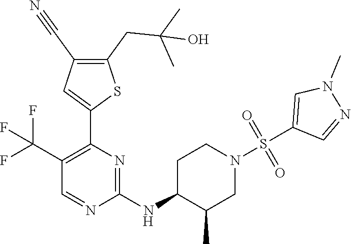 | LCMS calculated for C₂₄H₂₉F₃N₇O₃S₂ (M + H)⁺: m/z = 584.17; Found 584.2. |
| | 2-(2-hydroxy-2-methylpropyl)-5-(2- | |
| | (((3R,4S)-3-methyl-1-((1-methyl-1H- | |
| | pyrazol-4-yl)sulfonyl)piperidin-4- | |
| | yl)amino)-5-(trifluoromethyl)pyrimidin- | |
| | 4-yl)thiophene-3-carbonitrile | |
| 39 | 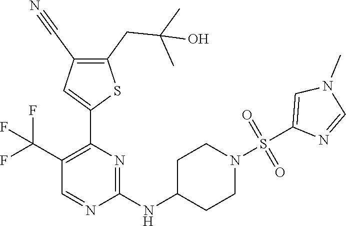 | LCMS calculated for C₂₃H₂₇F₃N₇O₃S₂ (M + H)⁺: m/z = 570.16; Found 570.2. |
| | 2-(2-hydroxy-2-methylpropyl)-5-(2-((1- | |
| | ((1-methyl-1H-imidazol-4-yl)sulfonyl) | |
| | piperidin-4-yl)amino)-5-(trifluoro- | |
| | methyl)pyrimidin-4-yl)thiophene-3- | |
| | carbonitrile | |
| 40 | 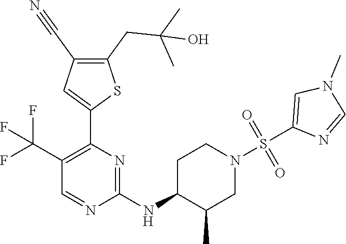 | LCMS calculated for C₂₄H₂₉F₃N₇O₃S₂ (M + H)⁺: m/z = 584.17; Found 584.2 |
| | 2-(2-hydroxy-2-methylpropyl)-5-(2- | |
| | (((3R,4S)-3-methyl-1-((1-methyl-1H- | |
| | imidazol-4-yl)sulfonyl)piperidin-4- | |
| | yl)amino)-5-(trifluoromethyl)pyrimidin- | |
| | 4-yl)thiophene-3-carbonitrile | |
| 41 | 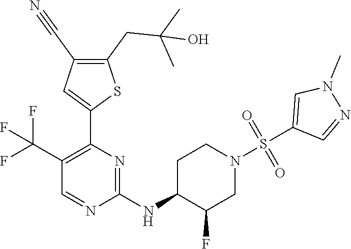 | LCMS calculated for C₂₃H₂₆F₄N₇O₃S₂ (M + H)⁺: m/z = 588.15; Found 588.2. |
| | 5-(2-(((3R,4S)-3-fluoro-1-((1-methyl-1H- | |
| | pyrazol-4-yl)sulfonyl)piperidin-4- | |
| | yl)amino)-5-(trifluoromethyl)pyrimidin- | |
| | 4-yl)-2-(2-hydroxy-2-methylpropyl) | |
| | thiophene-3-carbonitrile | |
| 42 | 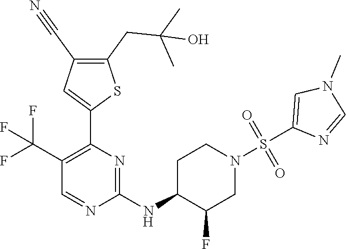 | LCMS calculated for C₂₃H₂₆F₄N₇O₃S₂ (M + H)⁺: 588.15; Found 588.2. ¹H NMR (300 MHz, DMSO-d₆) δ 8.69 (d, J = 7.8 Hz, 1H), 8.42 (d, J = 7.4 Hz, 1H), 7.84 (d, J = 3.1 Hz, 2H), 7.75 (d, J = 15.5 Hz, 1H), 4.94 (dd, J = 49.8, 26.7 Hz, 1H), 4.91(br, 1H), 4.20-3.82 (m, 2H), 3.72 (s, 3H), 3.69-3.62 (m, 1H), 3.09 (s, 2H), 2.86 (dd, J = 37.3, 13.4 Hz, 1H), 2.75- 2.59 (m, 1H), 2.09-1.92 (m, 1H), 1.89- 1.69 (m, 1H), 1.17 (s, 6H). |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)-2-(2-hydroxy-2-methylpropyl) | |
| | thiophene-3-carbonitrile | |
| 43 | 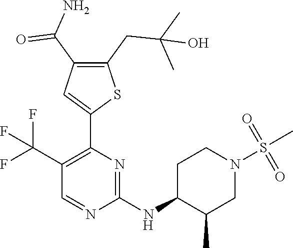 | LCMS calculated for C₂₁H₂₉F₃N₅O₄S₂ (M + H)⁺: m/z = 536.16; Found: 536.1. |
| | 2-(2-hydroxy-2-methylpropyl)-5-(2- | |
| | (((3R,4S)-3-methyl-1-(methylsulfonyl) | |
| | piperidin-4-yl)amino)-5-(trifluoro- | |
| | methyl)pyrimidin-4-yl)thiophene-3- | |
| | carboxamide | |
| 44 | 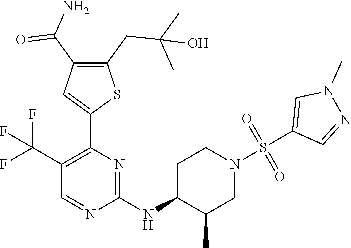 | LCMS calculated for C₂₄H₃₁F₃N₇O₄S₂ (M + H)⁺: m/z = 602.18; Found 602.2. |
| | 2-(2-hydroxy-2-methylpropyl)-5-(2- | |
| | (((3R,4S)-3-methyl-1-((1-methyl-1H- | |
| | pyrazol-4-yl)sulfonyl)piperidin-4- | |
| | yl)amino)-5-(trifluoromethyl)pyrimidin- | |
| | 4-yl)thiophene-3-carboxamide | |
| 45 | 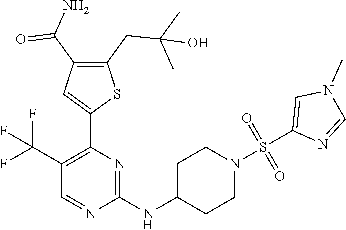 | LCMS calculated for C₂₃H₂₉F₃N₇O₄S₂ (M + H)⁺: m/z = 588.17; Found 588.2. |
| | 2-(2-hydroxy-2-methylpropyl)-5-(2-((1- | |
| | ((1-methyl-1H-imidazol-4-yl)sulfonyl) | |
| | piperidin-4-yl)amino)-5-(trifluoro- | |
| | methyl)pyrimidin-4-yl)thiophene-3- | |
| | carboxamide | |
| 46 | 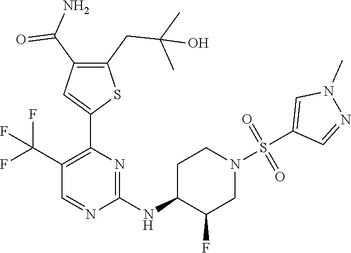 | LCMS calculated for C₂₃H₂₈F₄N₇O₄S₂ (M + H)⁺: m/z = 606.16; Found 606.2. ¹H NMR (300 MHz, DMSO-d₆) δ 8.64 (d, J = 9.9 Hz, 1H), 8.47-8.23 (m, 2H), 8.07 (s, 1H), 7.80 (d, J = 5.5 Hz, 1H), 7.75 (s, 1H), 7.38 (s, 1H), 5.13 (s, 1H), 4.99 (dd, J = 49.4, 36.2 Hz, 1H), 4.17-3.96 (m, 1H), 3.91 (d, J = 2.1 Hz, 3H), 3.89- 3.74 (m, 1H), 3.69-3.55 (m, 1H), 3.22 (d, J = 4.2 Hz, 2H), 2.82-2.54 (m, 2H), 2.05-1.94 (m, 1H), 1.91-1.71 (m, 1H), 1.17 (s, 6H). |
| | 5-(2-(((3R,4S)-3-fluoro-1-((1-methyl-1H- | |
| | pyrazol-4-yl)sulfonyl)piperidin-4- | |
| | yl)amino)-5-(trifluoromethyl)pyrimidin- | |
| | 4-yl)-2-(2-hydroxy-2-methylpropyl) | |
| | thiophene-3-carboxamide | |
| 47 | 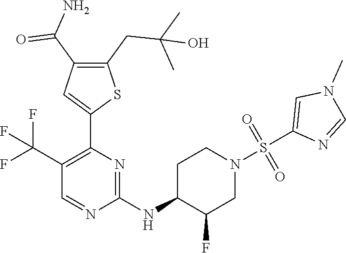 | LCMS calculated for C₂₃H₂₈F₄N₇O₄S₂ (M + H)⁺: m/z = 606.16; Found 606.2. ¹H NMR (300 MHz, DMSO-d₆) δ 8.64 (d, J = 7.1 Hz, 1H), 8.30 (dd, J = 16.3, 7.0 Hz, 1H), 8.08 (d, J = 5.6 Hz, 1H), 7.85 (s, 2H), 7.76 (d, J = 2.5 Hz, 1H), 7.39 (d, J = 7.0 Hz, 1H), 4.96 (dd, J = 48.2, 31.7 Hz, 1H), 4.05-3.82 (m, 2H), 3.72 (s, 3H), 3.69-3.64 (m, 1H), 3.22 (d, J = 4.6 Hz, 2H), 2.86 (dd, J = 36.7, 13.5 Hz, 1H), 2.74- 2.58 (m, 1H), 2.06-1.91 (m, 1H), 1.89- 1.67 (m, 1H), 1.17 (s, 6H). |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)-2-(2-hydroxy-2-methylpropyl) | |
| | thiophene-3-carboxamide | |
| 48 | 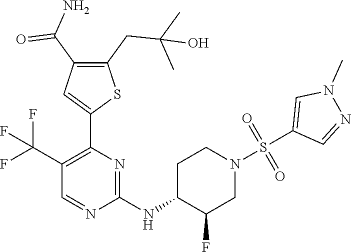 | LCMS calculated for C₂₃H₂₈F₄N₇O₄S₂ (M + H)⁺: m/z = 606.16; Found 606.2. ¹H NMR (300 MHz, DMSO-d₆) δ 8.64 (s, 1H), 8.38 (s, 1H), 8.31 (dd, J = 17.1, 7.8 Hz, 1H), 8.08 (d, J = 16.0 Hz, 1H), 7.83 (s, 1H), 7.75 (d, J = 4.9 Hz, 1H), 7.39 (d, J = 6.3 Hz, 1H), 5.30-4.97 (m, 1H), 4.95- 4.55 (m, 1H), 4.32-4.05 (m, 1H), 3.92 (s, 3H), 3.77-3.45 (m, 1H), 3.23 (s, 2H), 3.06-2.61 (m, 2H), 2.14-1.94 (m, 1H), 1.82-1.60 (m, 1H), 1.17 (s, 6H). |
| | 5-(2-(((3R,4R)-3-fluoro-1-((1-methyl- | |
| | 1H-pyrazol-4-yl)sulfonyl)piperidin-4- | |
| | yl)amino)-5-(trifluoromethyl)pyrimidin- | |
| | 4-yl)-2-(2-hydroxy-2-methylpropyl) | |
| | thiophene-3-carboxamide | |
| 49 | 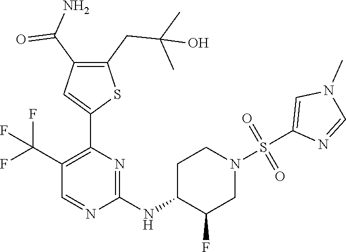 | LCMS calculated for C₂₃H₂₈F₄N₇O₄S₂ (M + H)⁺: m/z = 606.16; Found 606.2. ¹H NMR (300 MHz, DMSO-d₆) δ 8.64 (s, 1H), 8.31 (dd, J = 13.9, 7.6 Hz, 1H), 8.08 (d, J = 15.4 Hz, 1H), 7.87 (d, J = 1.8 Hz, 2H), 7.75 (d, J = 4.1 Hz, 1H), 7.38 (s, 1H), 4.91-4.51 (m, 1H), 4.24-3.98 (m, 2H), 3.73 (s, 3H), 3.54-3.38 (m, 1H), 3.23 (s, 2H), 3.07-2.70 (m, 2H), 2.05-1.91 (m, 1H), 1.75-1.57 (m, 1H), 1.17 (s, 6H). |
| | 5-(2-(((3R,4R)-3-fluoro-1-((1-methyl- | |
| | 1H-imidazol-4-yl)sulfonyl)piperidin-4- | |
| | yl)amino)-5-(trifluoromethyl)pyrimidin- | |
| | 4-yl)-2-(2-hydroxy-2-methylpropyl) | |
| | thiophene-3-carboxamide | |
| 50 | 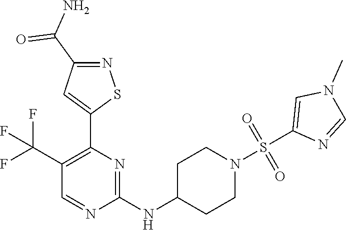 | LCMS calculated for C₁₈H₂₀F₃N₈O₃S₂ (M + H)⁺: m/z = 517.11; Found 517.0. ¹H NMR (300 MHz, DMSO-d₆) δ 8.76 (s, 1H), 8.42 (dd, J = 23.3, 7.2 Hz, 1H), 8.12 (d, J = 3.5 Hz, 1H), 8.06 (s, 1H), 7.90- 7.81 (m, 2H), 7.78 (s, 1H), 3.92-3.79 (m, 1H), 3.73 (s, 3H), 3.58-3.57 (m, 2H), 2.67 (dd, J = 26.3, 12.4 Hz, 2H), 2.08- 1.85 (m, 2H), 1.60 (dd, J = 21.7, 11.2 Hz, 2H). |
| | 5-(2-((1-((1-methyl-1H-imidazol-4- | |
| | yl)sulfonyl)piperidin-4-yl)amino)-5- | |
| | (trifluoromethyl)pyrimidin-4- | |
| | yl)isothiazole-3-carboxamide | |
| 51 | 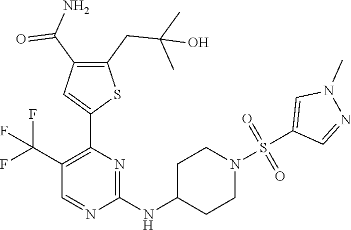 | LCMS calculated for C₂₃H₂₉F₃N₇O₄S₂ (M + H)⁺: m/z = 588.17; Found 588.2 |
| | 2-(2-hydroxy-2-methylpropyl)-5-(2-((1- | |
| | ((1-methyl-1H-pyrazol-4-yl)sulfonyl) | |
| | piperidin-4-yl)amino)-5-(trifluoro- | |
| | methyl)pyrimidin-4-yl)thiophene-3- | |
| | carboxamide | |
| 52 | 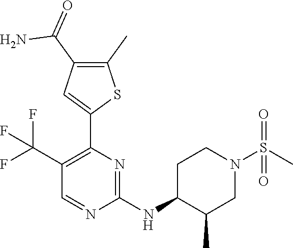 | LCMS calc. for C₁₈H₂₃F₃N₅O₃S₂ [M + H]⁺: m/z = 478.1; Found 478.1. ¹H NMR (300 MHz, DMSO) δ 8.64 (d, J = 10.6 Hz, 1H), 8.17 (dd, J = 34.2, 8.5 Hz, 1H), 7.76 (d, J = 4.8 Hz, 1H), 7.68 (s, 1H), 7.35 (s, 1H), 4.25-4.15 (m, 1H), 3.40- 3.04 (m, 4H), 2.87 (s, 3H), 2.65 (s, 3H), 2.26-2.16 (m, 1H), 1.90-1.71 (m, 2H), 0.96-0.81 (m, 3H). |
| | 2-methyl-5-(2-(((3R,4S)-3-methyl-1- | |
| | (methylsulfonyl)piperidin-4-yl)amino)-5- | |
| | (trifluoromethyl)pyrimidin-4- | |
| | yl)thiophene-3-carboxamide | |
| 53 | 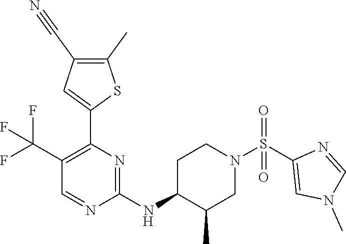 | LCMS calc. for C₂₁H₂₃F₃N₇O₂S₂ [M + H]⁺: m/z = 526.1; Found 526.2. ¹H NMR (300 MHz, DMSO) δ 8.66 (d, J = 5.9 Hz, 1H), 8.26 (dd, J = 30.2, 7.7 Hz, 1H), 7.87-7.78 (m, 2H), 7.72 (d, J = 10.3 Hz, 1H), 4.10-3.92 (m, 1H), 3.73 (s, 3H), 3.32-3.12 (m, 2H), 3.03-2.84 (m, 2H), 2.67 (s, 3H), 2.25-2.09 (m, 1H), 1.91-1.61 (m, 2H), 0.90 (t, J = 6.3 Hz, 3H). |
| | 2-methyl-5-(2-(((3R,4S)-3-methyl-1-((1- | |
| | methyl-1H-imidazol-4-yl)sulfonyl) | |
| | piperidin-4-yl)amino)-5-(trifluoro- | |
| | methyl)pyrimidin-4-yl)thiophene-3- | |
| | carbonitrile | |
| 54 | 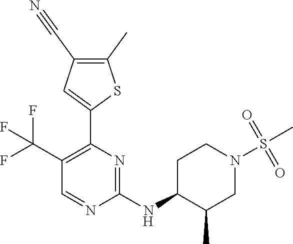 | LCMS calc. for C₁₈H₂₁F₃N₅O₂S₂ [M + H]⁺: m/z = 460.1; Found 460.0. ¹H NMR (300 MHz, DMSO) δ 8.70 (d, J = 8.6 Hz, 1H), 8.31 (dd, J = 38.4, 8.5 Hz, 1H), 7.74 (d, J = 11.4 Hz, 1H), 4.26-4.14 (m, 1H), 3.33-3.00 (m, 4H), 2.87 (d, J = 2.8 Hz, 3H), 2.69 (s, 3H), 2.25-2.12 (m, 1H), 1.89-1.71 (m, 2H), 0.94-0.85 (m, 3H). |
| | 2-methyl-5-(2-(((3R,4S)-3-methyl-1- | |
| | (methylsulfonyl)piperidin-4-yl)amino)-5- | |
| | (trifluoromethyl)pyrimidin-4- | |
| | yl)thiophene-3-carbonitrile | |
| 55 | 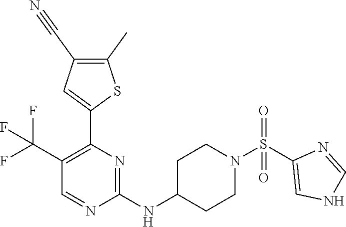 | LCMS calc. for C₁₉H₁₉F₃N₇O₂S₂ [M + H]⁺: m/z = 498.1; Found 498.0. ¹H NMR (300 MHz, DMSO) δ 8.65 (d, J = 5.5 Hz, 1H), 8.24 (dd, J = 23.7, 7.6 Hz, 1H), 7.92 (d, J = 3.7 Hz, 1H), 7.80 (s, 1H), 7.72 (d, J = 8.1 Hz, 1H), 3.87-3.65 (m, 1H), 3.65-3.51 (m, 2H), 2.76-2.56 (m, 5H), 2.00-1.81 (m, 2H), 1.68-1.49 (m, 2H). |
| | 5-(2-((1-((1H-imidazol-4-yl)sulfonyl) | |
| | piperidin-4-yl)amino)-5-(trifluoro- | |
| | methyl)pyrimidin-4-yl)-2-methyl- | |
| | thiophene-3-carbonitrile | |
| 56 | 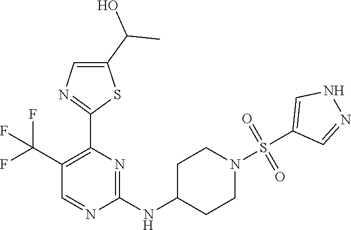 | LCMS calc. for C₁₈H₂₁F₃N₇O₃S₂ [M + H]⁺: m/z = 504.1; found 504.0. ¹H NMR (300 MHz, DMSO) δ 8.69 (s, 1H), 8.30-8.05 (m, 3H), 7.90 (s, 1H), 5.05 (q, J = 6.3 Hz, 1H), 3.80-3.75 (m, 1H), 3.60-3.50 (m, 2H), 2.60-2.40 (m, 2H), 2.05-1.95 (m, 2H), 1.75-1.60 (m, 2H). 1.47 (d, J = 6.3 Hz, 3H). |
| | 1-(2-(2-((1-((1H-pyrazol-4- | |
| | yl)sulfonyl)piperidin-4-yl)amino)-5- | |
| | (trifluoromethyl)pyrimidin-4-yl)thiazol- | |
| | 5-yl)ethan-1-ol | |
| 57 | 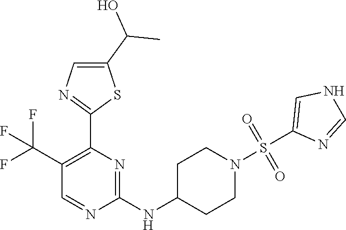 | LCMS calc. for C₁₈H₂₁F₃N₇O₃S₂ [M + H]⁺: m/z = 504.1; found 503.9. ¹H NMR (300 MHz, DMSO) δ 8.75-8.68 (m, 1H), 8.30-8.25 (m, 1H), 7.95-7.90 (m, 2H), 7.85-7.80 (m, 1H), 5.06 (q, J = 6 Hz, 1H), 3.85-3.80 (m, 1H), 3.65-3.55 (m, 2H), 2.75-2.55 (m, 2H), 2.05-1.95 (m, 2H), 1.60-1.45 (m, 2H), 1.47 (d, J = 6 Hz, 3H). |
| | 1-(2-(2-((1-((1H-imidazol-4- | |
| | yl)sulfonyl)piperidin-4-yl)amino)-5- | |
| | (trifluoromethyl)pyrimidin-4-yl)thiazol- | |
| | 5-yl)ethan-1-ol | |
| 58 | 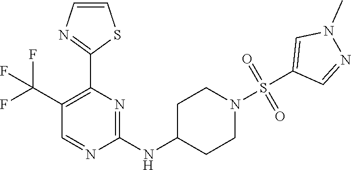 | LCMS calc. for C₁₇H₁₉F₃N₇O₂S₂ [M + H]⁺: m/z = 474.1; found 474.0. ¹H NMR (300 MHz, DMSO) δ 8.72 (s, 1H), 8.35-8.25 (m, 2H), 8.15-8.10 (m, 1H), 8.10-8.00 (m, 1H), 7.85-7.80 (m, 1H), 3.92 (s, 3H), 3.85-3.80 (m, 1H), 3.60-3.50 (m, 2H), 2.55-2.45 (m, 2H), 2.05-1.90 (m, 2H), 1.75-1.60 (m, 2H). |
| | N-(1-((1-methyl-1H-pyrazol-4- | |
| | yl)sulfonyl)piperidin-4-yl)-4-(thiazol-2- | |
| | yl)-5-(trifluoromethyl)pyrimidin-2-amine | |
| 59 | 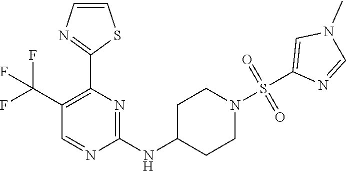 | LCMS calc. for C₁₇H₁₉F₃N₇O₂S₂ [M + H]⁺: m/z = 474.1; found 473.9. ¹H NMR (300 MHz, DMSO) δ 8.72 (s, 1H), 8.30-8.25 (m, 1H), 8.15-8.10 (m, 1H), 8.05-8.00 (m, 1H), 7.85-7.75 (m, 2H), 3.80-3.75 (m, 1H), 3.73 (s, 3H), 3.70-3.65 (m, 2H), 2.70-2.60 (m, 2H), 2.00-1.80 (m, 2H), 1.70-1.50 (m, 2H). |
| | N-(1-((1-methyl-1H-imidazol-4- | |
| | yl)sulfonyl)piperidin-4-yl)-4-(thiazol-2- | |
| | yl)-5-(trifluoromethyl)pyrimidin-2-amine | |
| 60 | 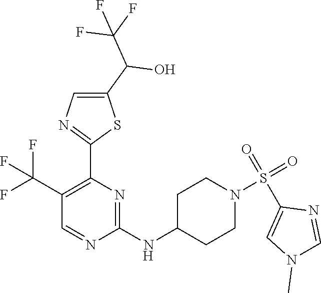 | LCMS calculated for C₁₉H₂₀F₆N₇O₃S₂ (M + H)⁺: m/z = 572.10; Found 572.0. ¹H NMR (300 MHz, DMSO) δ 8.74 (d, J = 2.7 Hz, 1H), 8.32 (dd, J = 23.2, 7.4 Hz, 1H), 8.14 (s, 1H), 7.83 (t, J = 3.6 Hz, 2H), 7.56 (t, J = 6.8 Hz, 1H), 5.81-5.67 (m, 1H), 3.80 (s, 1H), 3.72 (s, 3H), 3.59 (d, J = 12.4 Hz, 2H), 2.78-2.58 (m, 2H), 2.05- 1.87 (m, 2H), 1.60 (d, J = 11.4 Hz, 2H). |
| | 2,2,2-trifluoro-1-(2-(2-((1-((1-methyl- | |
| | 1H-imidazol-4-yl)sulfonyl)piperidin-4- | |
| | yl)amino)-5-(trifluoromethyl)pyrimidin- | |
| | 4-yl)thiazol-5-yl)ethan-1-ol | |
| 61 | 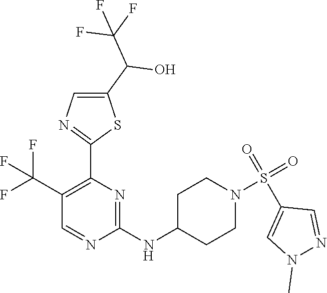 | LCMS calculated for C₁₉H₂₀F₆N₇O₃S₂ (M + H)⁺: m/z = 572.10; Found 572.0. ¹H NMR (300 MHz, DMSO) δ 8.74 (s, 1H), 8.42-8.25 (m, 2H), 8.14 (s, 1H), 7.80 (d, J = 4.2 Hz, 1H), 7.56 (t, J = 6.0 Hz, 1H), 5.81-5.67 (m, 1H), 3.92 (s, 3H), 3.83 (s, 1H), 3.51 (d, J = 12.2 Hz, 2H), 2.58 (d, J = 10.8 Hz, 1H), 2.43 (d, J = 10.3 Hz, 1H), 1.99 (t, J = 13.4 Hz, 2H), 1.66 (s, 2H). |
| | 2,2,2-trifluoro-1-(2-(2-((1-((1-methyl- | |
| | 1H-pyrazol-4-yl)sulfonyl)piperidin-4- | |
| | yl)amino)-5-(trifluoromethyl)pyrimidin- | |
| | 4-yl)thiazol-5-yl)ethan-1-ol | |
| 62 | 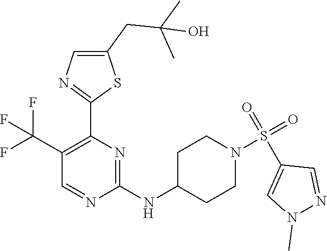 | LCMS calculated for C₂₁H₂₇F₃N₇O₃S₂ [M + H]⁺: m/z = 546.16; found 546.1. ¹H NMR (300 MHz, DMSO) δ 8.68 (s, 1H), 8.34 (d, J = 5.3 Hz, 1H), 8.24 (dd, J = 12.6, 7.6 Hz, 1H), 7.79 (d, J = 4.7 Hz, 2H), 4.72 (s, 1H), 3.92 (d, J = 2.9 Hz, 3H), 3.82 (s, 1H), 3.51 (d, J = 11.6 Hz, 2H), 2.95 (s, 2H), 2.48-2.33 (m, 2H), 1.99 (dd, J = 18.5, 11.0 Hz, 2H), 1.64 (d, J = 9.6 Hz, 2H), 1.12 (s, 6H). |
| | 2-methyl-1-(2-(2-((1-((1-methyl-1H- | |
| | pyrazol-4-yl)sulfonyl)piperidin-4- | |
| | yl)amino)-5-(trifluoromethyl)pyrimidin- | |
| | 4-yl)thiazol-5-yl)propan-2-ol | |
| 63 | 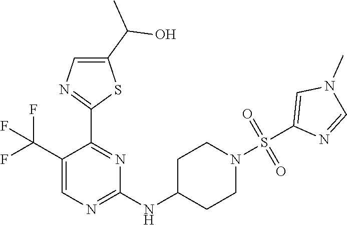 | LCMS calculated for C₁₉H₂₃F₃N₇O₃S₂ (M + H)⁺: m/z = 518.13; found 518.1. ¹H NMR (300 MHz, DMSO) δ 8.69 (d, J = 2.7 Hz, 1H), 8.24 (dd, J = 22.5, 7.3 Hz, 1H), 7.90 (s, 1H), 7.84 (t, J = 4.4 Hz, 2H), 5.06 (q, J = 6.3 Hz, 1H), 3.78 (d, J = 6.3 Hz, 1H), 3.73 (s, 3H), 3.54-3.45 (m, 2H), 2.76-2.55 (m, 2H), 2.09-1.87 (m, 2H), 1.61 (q, J = 10.3 Hz, 2H), 1.47 (d, J = 6.4 Hz, 3H). |
| | 1-(2-(2-((1-((1-methyl-1H-imidazol-4- | |
| | yl)sulfonyl)piperidin-4-yl)amino)-5- | |
| | (trifluoromethyl)pyrimidin-4-yl)thiazol- | |
| | 5-yl)ethan-1-ol | |
| 64 | 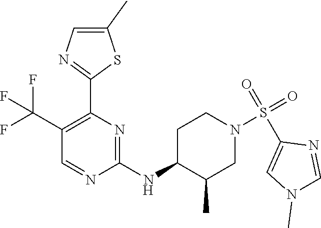 | LCMS calculated for C₁₉H₂₃F₃N₇O₂S₂ (M + H)⁺: m/z = 502.13; Found 502.2. ¹H NMR (300 MHz, DMSO) δ 8.68 (s, 1H), 8.24 (dd, J = 25.5, 7.4 Hz, 1H), 7.86 (s, 1H), 7.81 (s, 2H), 4.00 (s, 1H), 3.73 (s, 3H), 3.40-3.15 (m, 2H), 2.93 (dd, J = 29.1, 11.5 Hz, 2H), 2.52 (s, 3H), 2.24 (s, 1H), 1.95-1.82 (m, 1H), 1.73 (d, J = 12.6 Hz, 1H), 0.91 (d, J = 6.6 Hz, 3H). |
| | N-((3R,4S)-3-methyl-1-((1-methyl-1H- | |
| | imidazol-4-yl)sulfonyl)piperidin-4-yl)-4- | |
| | (5-methylthiazol-2-yl)-5- | |
| | (trifluoromethyl)pyrimidin-2-amine | |
| 65 | 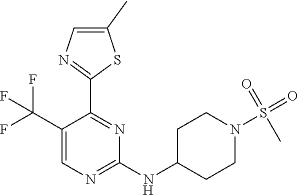 | LCMS calculated for C₁₅H₁₉F₃N₅O₂S₂ (M + H)⁺: m/z = 422.09; Found 422.0. ¹H NMR (300 MHz, DMSO-d₆) δ 8.72 (d, J = 11.0 Hz, 1H), 8.27 (dd, J = 25.1, 7.6 Hz, 1H), 7.87-7.80 (m, 1H), 3.94 (d, J = 16.4 Hz, 1H), 3.64-3.53 (m, 2H), 2.90 (d, J = 7.0 Hz, 5H), 2.54 (d, J = 1.1 Hz, 3H), 2.09-1.91 (m, 2H), 1.72-1.53 (m, 2H). |
| | N-(1-(methylsulfonyl)piperidin-4-yl)-4- | |
| | (5-methylthiazol-2-yl)-5- | |
| | (trifluoromethyl)pyrimidin-2-amine | |
| 66 | 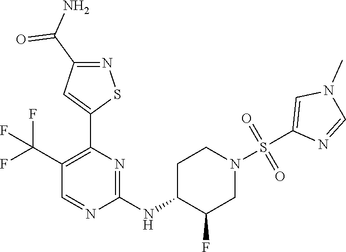 | LCMS calculated for C₁₈H₁₉F₄N₈O₃S₂ (M + H)⁺: m/z = 535.10; Found 535.0. ¹H NMR (300 MHz, DMSO-d₆) δ 8.80 (s, 1H), 8.59 (dd, J = 22.3, 8.0 Hz, 1H), 8.13 (s, 1H), 8.07 (s, 1H), 7.87 (d, J = 2.5 Hz, 2H), 7.79 (s, 1H), 4.88-4.53 (m, 1H), 4.26-4.00 (m, 1H), 3.73 (s, 3H), 4.72- 4.62 (m, 1H), 3.56-3.39 (m, 1H), 3.06- 2.70 (m, 2H), 2.13-1.93 (m, 1H), 1.77- 1.52 (m, 1H). |
| | 5-(2-(((3R,4R)-3-fluoro-1-((1-methyl- | |
| | 1H-imidazol-4-yl)sulfonyl)piperidin-4- | |
| | yl)amino)-5-(trifluoromethyl)pyrimidin- | |
| | 4-yl)isothiazole-3-carboxamide | |
| 67 | 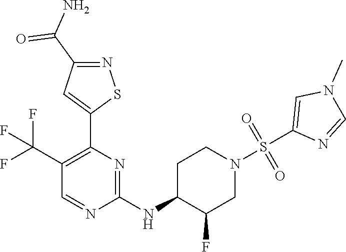 | LCMS calculated for C₁₈H₁₉F₄N₈O₃S₂ (M + H)⁺: m/z = 535.10; Found 535.0. ¹H NMR (300 MHz, DMSO-d₆) δ 8.80 (d, J = 10.2 Hz, 1H), 8.59 (dd, J = 19.1, 7.3 Hz, 1H), 8.12 (d, J = 10.8 Hz, 1H), 8.07 (s, 1H), 7.85 (d, J = 4.5 Hz, 1H), 7.79 (s, 1H), 4.95 (dd, J = 48.0, 22.5 Hz, 1H), 3.95-3.82 (m, 2H), 3.73 (s, 3H), 3.70- 3.62 (m, 1H), 2.90 (ddd, J = 23.9, 13.1, 10.1 Hz, 1H), 2.68 (dd, J = 21.7, 10.3 Hz, 1H), 2.10-1.93 (m, 1H), 1.81 (t, J = 12.6 Hz, 1H). |
| | 5-(2-(((3R,4S)-3-fluoro-1-((1-methyl-1H- | |
| | imidazol-4-yl)sulfonyl)piperidin-4- | |
| | yl)amino)-5-(trifluoromethyl)pyrimidin- | |
| | 4-yl)isothiazole-3-carboxamide | |
| 68 | 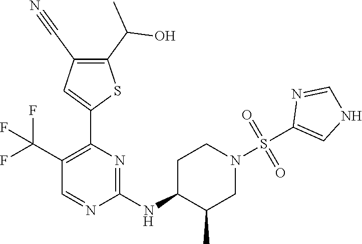 | LCMS calculated for C₂₁H₂₃F₃N₇O₃S₂ (M + H)⁺: m/z = 542.13; Found 542.1. |
| | 5-(2-(((3R,4S)-1-((1H-imidazol-4- | |
| | yl)sulfonyl)-3-methylpiperidin-4- | |
| | yl)amino)-5-(trifluoromethyl)pyrimidin- | |
| | 4-yl)-2-(1-hydroxyethyl)thiophene-3- | |
| | carbonitrile | |
| 85 | 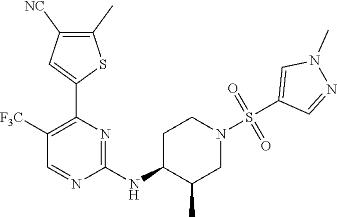 | LCMS calculated for C₂₁H₂₃F₃N₇O₃S₂ [M + H]⁺: m/z = 526.1; Found 526.0. |
| | 2-methyl-5-(2-(((3R,4S)-3-methyl-1-((1- | |
| | methyl-1H-pyrazol-4- | |
| | yl)sulfonyl)piperidin-4-yl)amino)-5- | |
| | (trifluoromethyl)pyrimidin-4- | |
| | yl)thiophene-3-carbonitrile | |

### Example 69. 5-(2-((1-((1-Methyl-1H-pyrazol-4-yl)sulfonyl)piperidin-4-yl)amino)-5-(trifluoro-methyl)pyrimidin-4-yl)thiophene-3-carbonitrile

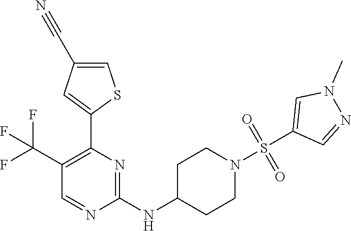 [SMILES: Cn1cc(S(=O)(=O)N2CCC(Nc3ncc(C(F)(F)F)c(-c4cc(C#N)cs4)n3)CC2)cn1]

### Step 1. 5-(2-((1-((1-Methyl-1H-pyrazol-4-yl)sulfonyl)piperidin-4-yl)amino)-5-(trifluoromethyl)pyrimidin-4-yl)thiophene-3-carbonitrile

A mixture of 5-bromothiophene-3-carbonitrile (16.1 mg, 0.09 mmol) (Intermediate 11), Pd(PPh₃)₄ (19.7 mg, 0.02 mmol), 4-chloro-N-[1-(1-methylpyrazol-4-yl)sulfonylpiperidin-4-yl]-5-(trifluoromethyl)pyrimidin-2-amine (Intermediate 24) (39.9 mg, 0.09 mmol), hexamethylditin (0.04 mL, 0.17 mmol) in 1,4-Dioxane (20.0 mL) was bubbled through N₂ gas for 5 min. The mixture was then stirred at 100° C. for 12 h. LCMS showed that the starting material was consumed. The reaction was then cooled down and KF solution (5.00 mL) was added. The mixture was stirred at RT for 1 h. The solvent was then removed under reduced pressure. The residue was added then extracted with ethyl acetate (10.0 mL×2). The combined organic layers were dried over Na₂SO₄, filtered, concentrated. The crude was purified by prep-HPLC on C₁₈ column (30×250 mm, 10 m) using mobile phase 15-70% MeCN/H₂O to afford the title compound (13.3 mg, 30.9% yield). LCMS calc. for C₁₉H₁₉F₃N₇O₂S₂ [M+H]⁺: m/z=498.1; Found 498.1. ¹H NMR (300 MHz, DMSO) δ 8.82 (d, J=3.8 Hz, 1H), 8.68 (s, 1H), 8.31 (dd, J=16.1, 5.8 Hz, 2H), 7.86 (d, J=9.2 Hz, 1H), 7.79 (d, J=5.1 Hz, 1H), 3.92 (s, 3H), 3.85 (s, 1H), 3.50 (d, J=8.1 Hz, 2H), 2.42 (d, J=12.7 Hz, 2H), 2.05-1.89 (m, 2H), 1.64 (dd, J=20.8, 10.2 Hz, 2H).

### Examples 70-86

Examples 70-86 shown below in Table 2 were prepared in accordance with the synthetic protocols set forth in Example 69 above using the appropriate intermediates, as well as commercial starting materials.

TABLE 2
Examples 70-86

| | | Analytical Data (LCMS and/or |
|---|---|---|
| Ex. | Molecular Structure and Name | NMR) |
| 70 | 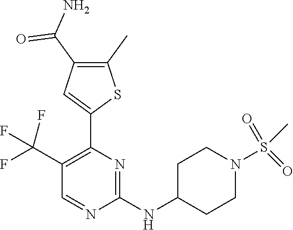 | LCMS calc. LCMS calc. for C₁₇H₂₁F₃N₅O₃S₂ [M + H]⁺: m/z = 464.1; found 463.8. |
| | 2-methyl-5-(2-((1-(methylsulfonyl)piperidin- | |
| | 4-yl)amino)-5-(trifluoromethyl)pyrimidin-4- | |
| | yl)thiophene-3-carboxamide | |
| 71 | 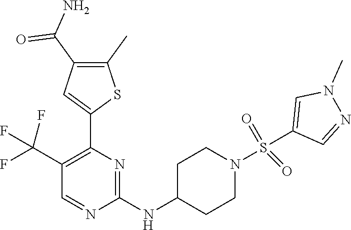 | LCMS calc. for C₂₀H₂₃F₃N₇O₃S₂ [M + H]⁺: m/z = 530.1; found 529.9. ¹H NMR (300 MHz, DMSO) δ 8.60 (s, 1H), 8.35-8.30 (m, 1H), 8.20-8.05 (m, 1H), 7.80-7.70 (m, 2H), 7.67 (s, 1H), 7.34 (s, 1H), 3.92 (s, 3H), 3.80-3.75 (m, 1H), 3.50-3.40 (m, 2H), 2.64 (s, 3H), 2.55-2.40 (m, 2H), 2.00-1.80 (m, 2H), 1.70-1.60 (m, 2H). |
| | 2-methyl-5-(2-((1-((1-methyl-1H-pyrazol-4- | |
| | yl)sulfonyl)piperidin-4-yl)amino)-5- | |
| | (trifluoromethyl)pyrimidin-4-yl)thiophene-3- | |
| | carboxamide | |
| 72 | 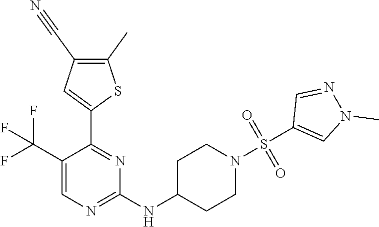 | LCMS calc. for C₂₀H₂₁F₃N₇O₂S₂ [M + H]⁺: m/z = 512.1; Found 512.1. ¹H NMR (300 MHz, DMSO) δ 8.65 (s, 1H), 8.34 (d, J = 6.5 Hz, 1H), 8.26 (dd, J = 24.3, 7.4 Hz, 1H), 7.79 (d, J = 6.6 Hz, 1H), 7.72 (d, J = 8.4 Hz, 1H), 3.92 (d, J = 2.3 Hz, 3H), 3.85-3.70 (m, 1H), 3.54-3.42 (m, 2H), 2.67 (d, J = 2.4 Hz, 3H), 2.49-2.37 (m,x65 2H), 2.02-1.82 (m, 2H), 1.71-1.56 (m, 2H). |
| | 2-methyl-5-(2-((1-((1-methyl-1H-pyrazol-4- | |
| | yl)sulfonyl)piperidin-4-yl)amino)-5- | |
| | (trifluoromethyl)pyrimidin-4-yl)thiophene-3- | |
| | carbonitrile | |
| 73 | 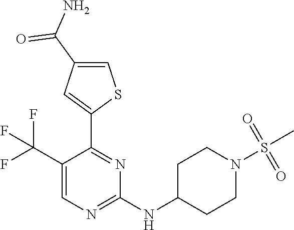 | LCMS calc. for C₁₆H₁₉F₃N₅O₃S₂ [M + H]⁺: m/z = 450.1; Found 450.0. ¹H NMR (300 MHz, DMSO) δ 8.67 (d, J = 12.8 Hz, 1H), 8.42 (d, J = 8.6 Hz, 1H), 8.21 (dd, J = 22.3, 7.5 Hz, 1H), 8.00 (d, J = 12.1 Hz, 2H), 7.38 (s, 1H), 3.89-3.79 (m, 1H), 3.62-3.51 (m, 2H), 2.96-2.79 (m, 5H), 2.26- 1.80 (m, 2H), 1.71-1.50 (m, 2H). |
| | 5-(2-((1-(methylsulfonyl)piperidin-4- | |
| | yl)amino)-5-(trifluoromethyl)pyrimidin-4- | |
| | yl)thiophene-3-carboxamide | |
| 73 | 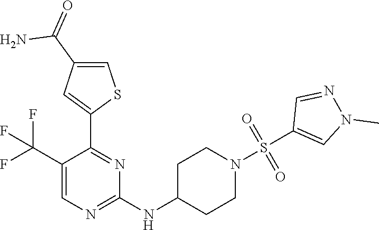 | LCMS calc. for C₁₉H₂₁F₃N₇O₃S₂ [M + H]⁺: m/z = 516.1; Found 516.0. ¹H NMR (300 MHz, DMSO) δ 8.64 (s, 1H), 8.41 (s, 1H), 8.34 (d, J = 6.0 Hz, 1H), 8.19 (dd, J = 18.9, 7.4 Hz, 1H), 7.98 (d, J = 8.4 Hz, 2H), 7.80 (d, J = 7.3 Hz, 1H), 7.37 (s, 1H), 3.92 (d, J = 2.7 Hz, 3H), 3.87-3.70 (m, 1H), 3.53- 3.42 (m, 2H), 2.50-2.38 (m, 2H), 2.07- 1.89 (m, 2H), 1.73-1.53 (m, 2H). |
| | 5-(2-((1-((1-methyl-1H-pyrazol-4- | |
| | yl)sulfonyl)piperidin-4-yl)amino)-5- | |
| | (trifluoromethyl)pyrimidin-4-yl)thiophene-3- | |
| | carboxamide | |
| 75 | 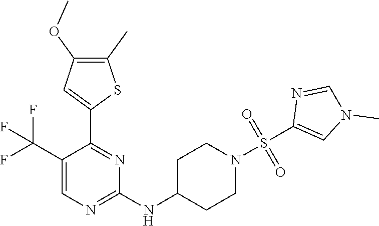 | LCMS calculated for C₂₀H₂₄F₃N₆O₃S₂ (M + H)⁺: m/z = 517.13; Found 517.1. |
| | 4-(4-methoxy-5-methylthiophen-2-yl)-N-(1- | |
| | ((1-methyl-1H-imidazol-4-yl)sulfonyl) | |
| | piperidin-4-yl)-5-(trifluoromethyl)pyrimidin- | |
| | 2-amine | |
| 76 | 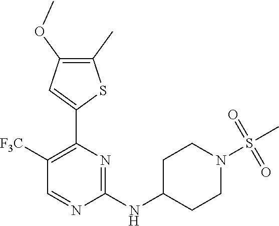 | LCMS calculated for C₁₇H₂₂F₃N₄O₃S₂ (M + H)⁺: m/z = 451.11; Found 451.1. ¹H NMR (300 MHz, DMSO-d₆) δ 8.60 (d, J = 14.3 Hz, 1H), 8.08 (dd, J = 26.1, 7.8 Hz, 1H), 7.44 (d, J = 5.5 Hz, 1H), 4.04-3.84 (m, 1H), 3.80 (s, 3H), 3.56 (d, J = 11.9 Hz, 2H), 3.00-2.75 (m, 5H), 2.28 (s, 3H), 2.11-1.89 (m, 2H), 1.60 (dd, J = 22.9, 11.2 Hz, 2H). |
| | 4-(4-methoxy-5-methylthiophen-2-yl)-N-(1- | |
| | (methylsulfonyl)piperidin-4-yl)-5- | |
| | (trifluoromethyl)pyrimidin-2-amine | |
| 77 | 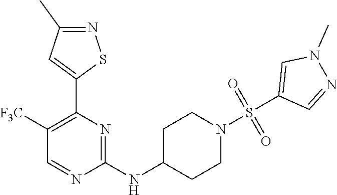 | LCMS calculated for C₁₈H₂₁F₃N₇O₂S₂ (M + H)⁺: m/z = 488.12; Found 488.0. ¹H NMR (300 MHz, DMSO-d₆) δ 8.71 (s, 1H), 8.42-8.26 (m, 2H), 7.79 (d, J = 6.2 Hz, 1H), 7.56 (d, J = 5.1 Hz, 1H), 3.91 (d, J = 2.2 Hz, 3H), 3.78 (dd, J = 34.1, 8.8 Hz, 1H), 3.53-3.41 (m, 4H), 2.49 (s, 3H), 1.96 (t, J = 14.7 Hz, 2H), 1.62 (dt, J = 14.0, 7.2 Hz, 2H). |
| | N-(1-((1-methyl-1H-pyrazol-4-yl)sulfonyl) | |
| | piperidin-4-yl)-4-(3-methylisothiazol-5-yl)- | |
| | 5-(trifluoromethyl)pyrimidin-2-amine | |
| 78 | 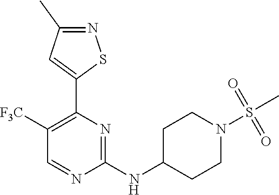 | LCMS calculated for C₁₅H₁₉F₃N₅O₂S₂ (M + H)⁺: m/z = 422.09; Found 422.0. |
| | 4-(3-methylisothiazol-5-yl)-N-(1-(methyl- | |
| | sulfonyl)piperidin-4-yl)-5-(trifluoromethyl) | |
| | pyrimidin-2-amine | |
| 79 | 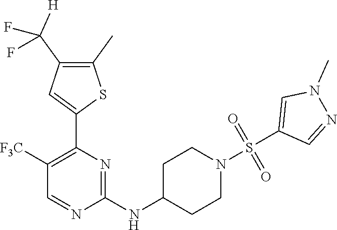 | LCMS calculated for C₂₀H₂₂F₅N₆O₂S₂ (M + H)⁺: m/z = 537.12; Found 537.2. ¹H NMR (300 MHz, DMSO-d₆) δ 8.62 (d, J = 2.1 Hz, 1H), 8.34 (d, J = 7.6 Hz, 1H), 8.17 (dd, J = 25.4, 7.5 Hz, 1H), 7.80 (d, J = 7.8 Hz, 1H), 7.63 (d, J = 3.9 Hz, 1H), 7.15 (t, J = 54.8 Hz, 1H), 3.92 (d, J = 2.8 Hz, 3H), 3.79 (s, 1H), 3.47 (d, J = 12.1 Hz, 2H), 2.56 (t, J = 1.7 Hz, 3H), 2.40 (d, J = 11.5 Hz, 2H), 1.97 (t, J = 16.3 Hz, 2H), 1.66 (t, J = 11.3 Hz, 2H). |
| | 4-(4-(difluoromethyl)-5-methylthiophen-2- | |
| | yl)-N-(1-((1-methyl-1H-pyrazol-4- | |
| | yl)sulfonyl)piperidin-4-yl)-5- | |
| | (trifluoromethyl)pyrimidin-2-amine | |
| 80 | 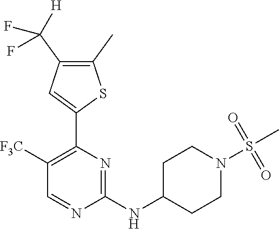 | LCMS calculated for C₁₇H₂₀F₅N₄O₂S₂ (M + H)⁺: m/z = 471.09; Found 471.2. ¹H NMR (300 MHz, DMSO-d₆) δ 8.65 (d, J = 12.7 Hz, 1H), 8.19 (dd, J = 27.8, 7.6 Hz, 1H), 7.65 (d, J = 7.7 Hz, 1H), 7.16 (t, J = 54.8 Hz, 1H), 4.03- 3.82 (m, 1H), 3.58 (s, 2H), 2.89 (d, J = 7.8 Hz, 5H), 2.57 (d, J = 1.8 Hz, 3H), 1.99 (t, J = 15.9 Hz, 2H), 1.60 (q, J = 10.7 Hz, 2H). |
| | 4-(4-(difluoromethyl)-5-methylthiophen-2- | |
| | yl)-N-(1-(methylsulfonyl)piperidin-4-yl)-5- | |
| | (trifluoromethyl)pyrimidin-2-amine | |
| 81 | 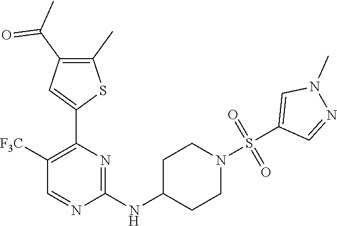 | LCMS calc. for C₂₁H₂₄F₃N₆O₃S₂ [M + H]⁺: m/z = 529.1; Found 529.1. ¹H NMR (300 MHz, DMSO) δ 8.62 (s, 1H), 8.34 (d, J = 6.9 Hz, 1H), 8.18 (dd, J = 22.5, 7.6 Hz, 1H), 7.94 (s, 1H), 7.79 (d, J = 7.5 Hz, 1H), 3.92 (s, 3H), 3.82-3.70 (m, 1H), 3.54-3.44 (m, 2H), 2.72 (s, 3H), 2.51 (d, J = 1.8 Hz, 3H), 2.52-2.44 (m, 2H), 2.05-1.90 (m, 2H), 1.72-1.57 (m, 2H). |
| | 1-(2-methyl-5-(2-((1-((1-methyl-1H-pyrazol- | |
| | 4-yl)sulfonyl)piperidin-4-yl)amino)-5- | |
| | (trifluoromethyl)pyrimidin-4-yl)thiophen-3- | |
| | yl)ethan-1-one | |
| 82 | 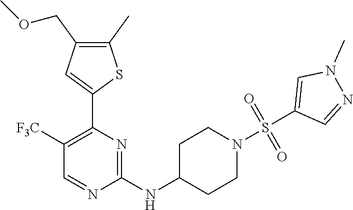 | LCMS calc. for C₂₁H₂₆F₃N₆O₃S₂ [M + H]⁺: m/z = 531.1; Found 531.2. ¹H NMR (300 MHz, DMSO) δ 8.56 (s, 1H), 8.34 (d, J = 8.3 Hz, 1H), 8.06 (dd, J = 25.1, 7.6 Hz, 1H), 7.80 (d, J = 8.7 Hz, 1H), 7.52 (d, J = 4.5 Hz, 1H), 4.34 (s, 2H), 3.92 (s, 3H), 3.80-3.70 (m, 1H), 3.55-3.37 (m, 2H), 3.26 (s, 3H), 2.38 (s, 3H), 2.50-2.27 (m, 2H), 2.04-1.89 (m, 2H), 1.72-1.57 (m, 2H). |
| | 4-(4-(methoxymethyl)-5-methylthiophen-2- | |
| | yl)-N-(1-((1-methyl-1H-pyrazol-4- | |
| | yl)sulfonyl)piperidin-4-yl)-5- | |
| | (trifluoromethyl)pyrimidin-2-amine | |
| 83 | 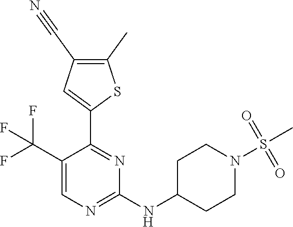 | LCMS calc. for C₁₇H₁₉F₃N₅O₂S₂ [M + H]⁺: m/z = 446.1; Found 446.0. ¹H NMR (300 MHz, DMSO) δ 8.69 (d, J = 12.5 Hz, 1H), 8.29 (dd, J = 27.2, 7.3 Hz, 1H), 7.74 (d, J = 10.9 Hz, 1H), 4.00-3.81 (m, 1H), 3.61-3.50 (m, 2H), 3.01-2.79 (m, 5H), 2.71 (d, J = 12.7 Hz, 3H), 2.03-1.82 (m, 2H), 1.68- 1.53 (m, 2H). |
| | 2-methyl-5-(2-((1-(methylsulfonyl)piperidin- | |
| | 4-yl)amino)-5-(trifluoromethyl)pyrimidin-4- | |
| | yl)thiophene-3-carbonitrile | |
| 84 | 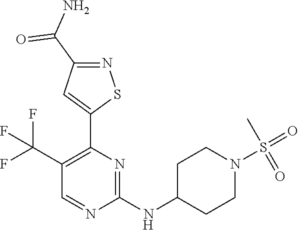 | LCMS calculated for C₁₅H₁₈F₃N₆O₃S₂ (M + H)⁺: m/z = 451.08; Found 451.0. ¹H NMR (300 MHz, DMSO-d₆) δ 8.80 (d, J = 9.0 Hz, 1H), 8.46 (dd, J = 24.4, 7.5 Hz, 1H), 8.12 (s, 1H), 8.08 (d, J = 4.3 Hz, 1H), 7.79 (s, 1H), 4.10-3.80 (m, 1H), 3.57 (d, J = 9.4 Hz, 2H), 3.05- 2.81 (m, 5H), 2.00 (t, J = 15.3 Hz, 2H), 1.61 (td, J = 15.2, 4.3 Hz, 2H). |
| | 5-(2-((1-(methylsulfonyl)piperidin-4- | |
| | yl)amino)-5-(trifluoromethyl)pyrimidin-4- | |
| | yl)isothiazole-3-carboxamide | |
| 86 | 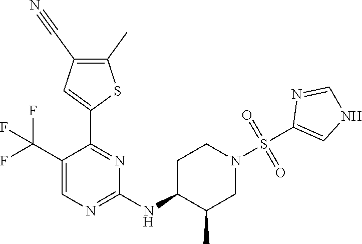 | LCMS calc. for C₂₀H₂₁F₃N₇O₂S₂ [M + H]⁺: m/z = 512.1; Found 512.0. |
| | 5-(2-(((3R,4S)-1-((1H-imidazol-4- | |
| | yl)sulfonyl)-3-methylpiperidin-4-yl)amino)- | |
| | 5-(trifluoromethyl)pyrimidin-4-yl)-2- | |
| | methylthiophene-3-carbonitrile | |

### Example 87. 5-(2-(((3R,4S)-1-((1-(Cyanomethyl)-1H-pyrazol-4-yl)sulfonyl)-3-methylpiperidin-4-yl)amino)-5-(trifluoromethyl)pyrimidin-4-yl)thiophene-3-carboxamide

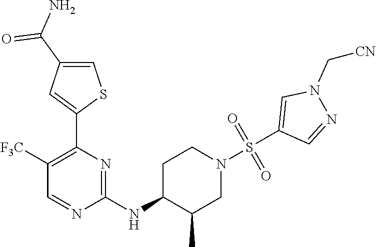 [SMILES: C[C@@H]1CN(S(=O)(=O)c2cnn(CC#N)c2)CC[C@@H]1Nc1ncc(C(F)(F)F)c(-c2cc(C(N)=O)cs2)n1]

### Step 1. Tert-butyl (3R,4S)-4-((4-(4-carbamoylthiophen-2-yl)-5-(trifluoromethyl)pyrimidin-2-yl)amino)-3-methylpiperidine-1-carboxylate

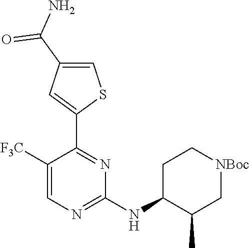 [SMILES: C[C@@H]1CN(C(=O)OC(C)(C)C)CC[C@@H]1Nc1ncc(C(F)(F)F)c(-c2cc(C(N)=O)cs2)n1]

A mixture of 5-bromothiophene-3-carboxamide (Intermediate 10) (64.0 mg, 0.310 mmol), Pd(PPh₃)₄ (71.8 mg, 60.0 μmol), tert-butyl (3R,4S)-4-[[4-chloro-5-(trifluoromethyl)-pyrimidin-2-yl]amino]-3-methylpiperidine-1-carboxylate (Intermediate 25) (122.6 mg, 0.310 mmol), hexamethylditin (0.13 mL, 0.620 mmol) in 1,4-dioxane (20.0 mL) was sparged with N₂ for 5 min. The mixture was then stirred at 100° C. in a sealed tube for 12 h. LCMS showed that the starting material was consumed. The reaction was then cooled to rt and KF (5 mL, 10% aq.) was added. The mixture was stirred at rt for 1 h. The solvent was then removed under reduced pressure. The residue was extracted with ethyl acetate (10.0 mL×2). The combined organic layers were dried over Na₂SO₄, filtered, concentrated. The crude was purified by prep-HPLC on C₁₈ column (10-70% MeCN in 0.1% TFA(aq), pH=2) to afford the title compound (104 mg, 51.7% yield). LCMS calc. for C₂₁H₂₇F₃N₅O₃S [M+H]⁺: m/z=486.2; Found 486.1.

### Step 2. 5-(2-(((3R,4S)-3-methylpiperidin-4-yl)amino)-5-(trifluoromethyl)pyrimidin-4-yl)thiophene-3-carboxamide

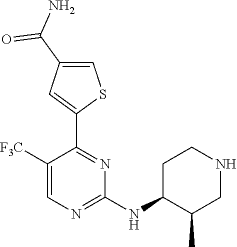 [SMILES: C[C@@H]1CNCC[C@@H]1Nc1ncc(C(F)(F)F)c(-c2cc(C(N)=O)cs2)n1]

To the solution of tert-butyl (3R,4S)-4-[[4-(4-carbamoylthiophen-2-yl)-5-(trifluoro-methyl)pyrimidin-2-yl]amino]-3-methylpiperidine-1-carboxylate (80.0 mg, 0.120 mmol) in DCM (4.00 mL) was added TFA (2.00 mL). The mixture was stirred at rt for 2 h. LCMS showed that the starting material was consumed. The reaction mixture was concentrated under reduced pressure to afford the title compound (60.2 mg, quantitative yield) as the TFA salt, which was directly used in the next step without further purification. LCMS calc. for C₁₆H₁₉F₃N₅OS [M+H]⁺: m/z=386.1; Found 386.1.

### Step 3: 5-(2-(((3R,4S)-1-((1H-pyrazol-4-yl)sulfonyl)-3-methylpiperidin-4-yl)amino)-5-(trifluoromethyl)pyrimidin-4-yl)thiophene-3-carboxamide

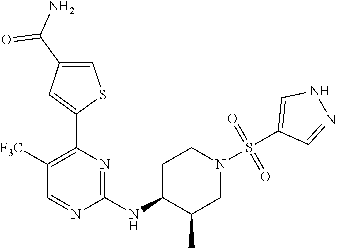 [SMILES: C[C@@H]1CN(S(=O)(=O)c2cn[nH]c2)CC[C@@H]1Nc1ncc(C(F)(F)F)c(-c2cc(C(N)=O)cs2)n1]

To a solution of 5-[2-[[(3R,4S)-3-methylpiperidin-4-yl]amino]-5-(trifluoromethyl)-pyrimidin-4-yl]thiophene-3-carboxamide (14.2 mg, 40.0 μmol) in DMA (1.00 mL) was added 1H-pyrazole-4-sulfonyl chloride (6.70 mg, 40.0 μmol) at 0° C., followed by the addition of DIPEA (47.4 mg, 0.370 mmol). The mixture was then stirred at rt for 3 h. LCMS showed the starting material was consumed. The crude was purified by prep-HPLC on C₁₈ column (10-60% MeCN in 0.1% TFA(aq), pH=2) to afford the title compound (8.60 mg, 45.0% yield). LCMS calc. for C₁₉H₂₁F₃N₇O₃S₂ [M+H]⁺: m/z=516.1; Found 516.0.

### Step 4: 5-(2-(((3R,4S)-1-((1-(cyanomethyl)-1H-pyrazol-4-yl)sulfonyl)-3-methylpiperidin-4-yl)amino)-5-(trifluoromethyl)pyrimidin-4-yl)thiophene-3-carboxamide

To a solution of 5-[2-[[(3R,4S)-3-methyl-1-(1H-pyrazol-4-ylsulfonyl)piperidin-4-yl]amino]-5-(trifluoromethyl)pyrimidin-4-yl]thiophene-3-carboxamide (8.60 mg, 17.0 μmol) in DMA (2.00 mL) was added chloroacetonitrile (1.20 mg, 17.0 μmol) and K₂CO₃ (6.8 mg, 50.0 μmol). The mixture was stirred at rt for 2 h. LCMS showed that the starting material was consumed. The crude was purified by prep-HPLC on C₁₈ column (10-60% MeCN in 0.1% TFA(aq), pH=2) to afford the title compound (6.90 mg, 74.0% yield). LCMS calc. for C₂₁H₂₂F₃N₈O₃S₂ [M+H]⁺: m/z=555.1; Found 555.1. ¹H NMR (300 MHz, DMSO) δ 8.64 (d, J=7.5 Hz, 1H), 8.46 (s, 1H), 8.40 (d, J=5.7 Hz, 1H), 8.22 (dd, J=22.7, 7.8 Hz, 1H), 8.05-7.95 (m, 3H), 7.37 (s, 1H), 5.58 (s, 2H), 4.20-4.00 (m, 1H), 3.25-2.75 (m, 4H), 2.30-2.15 (m, 1H), 1.95-1.70 (m, 2H), 0.92 (d, J=6.8 Hz, 3H).

### Example 88

Example 88 shown below in Table 3 was prepared in accordance with the synthetic protocols set forth in Example 87 above using the appropriate intermediates, as well as commercial starting materials.

TABLE 3
Example 88

| Ex. | Molecular Structure and Name | Analytical Data (LCMS and/or NMR) |
|---|---|---|
| 88 | 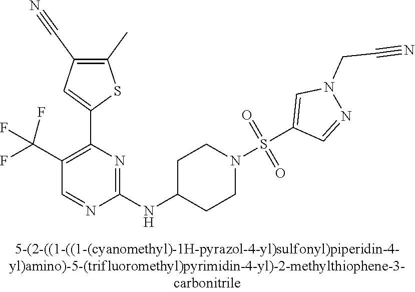 [SMILES: Cc1sc(-c2nc(NC3CCN(S(=O)(=O)c4cnn(CC#N)c4)CC3)ncc2C(F)(F)F)cc1C#N] | LCMS calc. for C₂₁H₂₀F₃N₈O₂S₂ [M + H]⁺: m/z = 537.1; found 537.1. ¹H NMR (300 MHz, DMSO) δ 8.65 (s, 1H), 8.50-8.45 (m, 1H), 8.35-8.25 (m, 1H), 8.00-7.95 (m, 1H), 7.75-7.70 (m, 1H), 5.65-5.52 (m, 2H), 3.85-3.75 (m, 1H), 3.60-3.50 (m, 2H), 2.66 (s, 3H), 2.60-2.40 (m, 2H), 2.00-1.90 (m, 2H), 1.70-1.55 (m, 2H). |

### Example 89. 1-(2-(2-((1-((1-Methyl-1H-pyrazol-4-yl)sulfonyl)piperidin-4-yl)amino)-5-(trifluoromethyl)pyrimidin-4-yl)thiazol-5-yl)ethan-1-ol

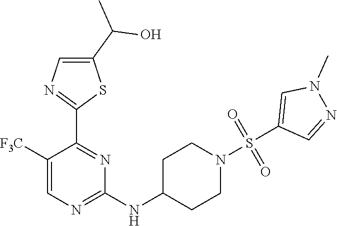 [SMILES: CC(O)c1cnc(-c2nc(NC3CCN(S(=O)(=O)c4cnn(C)c4)CC3)ncc2C(F)(F)F)s1]

### Step 1. 1-(2-(2-Chloro-5-(trifluoromethyl)-3,4-dihydropyrimidin-4-yl)thiazol-5-yl)ethan-1-ol

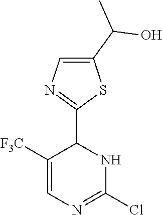 [SMILES: CC(O)c1cnc(C2NC(Cl)=NC=C2C(F)(F)F)s1]

To a solution of 1-(2-bromo-1,3-thiazol-5-yl)ethanol (Intermediate 6) (60.0 mg, 0.290 mmol) in THF (1.00 mL) was added n-butyllithium (0.290 mL, 0.720 mmol) dropwise at −78° C. The reaction mixture was stirred at −78° C. for 30 min. Then a solution of 2-chloro-5-(trifluoro-methyl)pyrimidine (63.0 mg, 0.350 mmol) in THF (0.500 mL) was added. The resulting mixture was further stirred at −78° C. for 30 min. LCMS showed the starting material was consumed. The reaction was quenched with saturated NH₄Cl solution (2.00 mL) and extracted with DCM (2.00 mL×3). The combined organic layers were dried over Na₂SO₄, filtered and concentrated to yield the title compound (88.0 mg, 97.6% yield), which was used for the next reaction without further purification. LCMS calculated for C₁₀H₁₀ClF₃N₃OS (M+H)⁺: m/z=312.02/314.02; Found 312.0/314.0.

### Step 2. 1-(2-(2-Chloro-5-(trifluoromethyl)pyrimidin-4-yl)thiazol-5-yl)ethan-1-ol

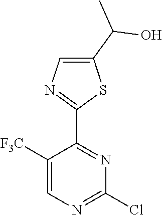 [SMILES: CC(O)c1cnc(-c2nc(Cl)ncc2C(F)(F)F)s1]

To a solution of 1-(2-(2-chloro-5-(trifluoromethyl)-3,4-dihydropyrimidin-4-yl)thiazol-5-yl)ethan-1-ol (88.0 mg, 0.280 mmol) in THF (1.00 mL) was added 2,3-dichloro-5,6-dicyano-p-benzoquinone (64.0 mg, 0.280 mmol). The reaction mixture was stirred at RT for 1 h. LCMS showed the starting material was consumed. The reaction was diluted with DCM (2.00 mL) and washed with 10% K₂CO₃ solution (2.00 mL), dried over Na₂SO₄, filtered and concentrated. The residue was purified by prep-HPLC on a C₁₈ column eluting with MeCN/H₂O (20-60%) with 0.1% TFA to yield the title compound (26.0 mg, 29.7% yield). ¹H NMR (300 MHz, CDCl₃) δ 9.00 (s, 1H), 7.98 (s, 1H), 5.29 (q, J=6.5 Hz, 1H), 1.69 (d, J=6.5 Hz, 3H). LCMS calculated for C₁₀H₈ClF₃N₃OS (M+H)⁺: m/z=310.00/312.00; found 310.0/312.0.

### Step 3. 1-(2-(2-((1-((1-Methyl-1H-pyrazol-4-yl)sulfonyl)piperidin-4-yl)amino)-5-(trifluoromethyl)pyrimidin-4-yl)thiazol-5-yl)ethan-1-ol

To a solution of 1-(2-(2-chloro-5-(trifluoromethyl)pyrimidin-4-yl)thiazol-5-yl)ethan-1-ol (17.0 mg, 0.0549 mmol) and 1-(1-methylpyrazol-4-yl)sulfonylpiperidin-4-amine (Intermediate 22) (16.1 mg, 0.0659 mmol) in NMP (1.00 mL) was added N,N-diisopropylethylamine (28.7 μL, 0.165 mmol). The reaction mixture was stirred at 110° C. for 2 h. LCMS showed the starting material was consumed. The reaction was diluted with MeOH (2.00 mL) and purified by prep-HPLC on a C₁₈ column eluting with MeCN/H₂O (20-60%) with 0.1% TFA to yield the title compound (22.2 mg, 77.8% yield). ¹H NMR (300 MHz, DMSO) δ 8.69 (s, 1H), 8.34 (d, J=6.4 Hz, 1H), 8.25 (dd, J=22.6, 7.3 Hz, 1H), 7.90 (s, 1H), 7.80 (d, J=6.6 Hz, 1H), 5.06 (q, J=6.3 Hz, 1H), 3.92 (s, 3H), 3.49 (t, J=12.7 Hz, 3H), 2.56 (d, J=11.5 Hz, 1H), 2.41 (d, J=10.2 Hz, 1H), 1.99 (t, J=14.3 Hz, 2H), 1.67 (s, 2H), 1.47 (d, J=6.4 Hz, 3H). LCMS calculated for C₁₉H₂₃F₃N₇O₃S₂ (M+H)⁺: m/z=518.13; Found 518.1.

### Example 90. 2-(1-Hydroxyethyl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile

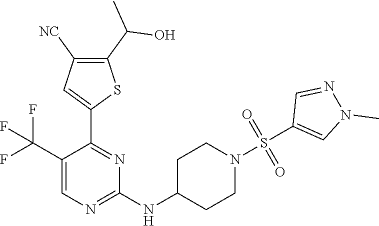 [SMILES: CC(O)c1sc(-c2nc(NC3CCN(S(=O)(=O)c4cnn(C)c4)CC3)ncc2C(F)(F)F)cc1C#N]

### Step 1. 2-(1-Hydroxyethyl)-5-(2-((1-((1-methyl-1H-pyrazol-4-yl)sulfonyl)piperidin-4-yl)amino)-5-(trifluoromethyl)pyrimidin-4-yl)thiophene-3-carboxamide

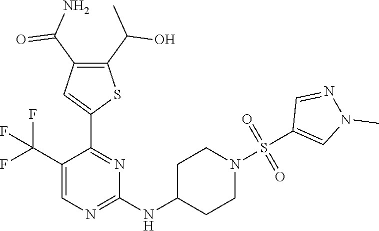 [SMILES: CC(O)c1sc(-c2nc(NC3CCN(S(=O)(=O)c4cnn(C)c4)CC3)ncc2C(F)(F)F)cc1C(N)=O]

A mixture of 5-bromo-2-(1-hydroxyethyl)thiophene-3-carboxamide (Intermediate 3) (25.0 mg, 0.100 mmol), 4-chloro-N-[1-(1-methylpyrazol-4-yl)sulfonylpiperidin-4-yl]-5-(trifluoro-methyl)pyrimidin-2-amine (Intermediate 24) (51.0 mg, 0.120 mmol) and Pd(PPh₃)₄ (23.1 mg, 20.0 μmol) was added 1,4-dioxane (20.0 mL). The mixture was purged with N₂ for 2 mins. Then hexamethylditin (60.0 μL, 0.300 mmol) was added. The reaction was degassed and refilled with N₂ for three times. Then it was stirred at 100° C. for 20 h. The LCMS showed full consumption of starting material. The reaction was cooled to RT then KF solution (0.3 N) was added and stirred for 2 h. The reaction was poured into brine and extracted with EtOAc (3×). The combined organic phase was dried over Na₂SO₄, filtered, and condensed. The residue was purified by prep-HPLC on C₁₈ column (30×250 mm, 10 μm) using mobile phase 30%-50% MeCN/H₂O (containing 0.1% TFA). The desired fractions were collected, neutralized to pH ˜7 with NaHCO₃ solution and concentrated. The residue was diluted by NaHCO₃ solution and extracted with EtOAc (3×). The combined organic phase was dried over Na₂SO₄, filtered and concentrated. The residue was resuspended in 1.0 mL of MeCN and diluted by water then freeze-dried to give the title compound (23.0 mg, 41.1% yield) as a white solid. LCMS calculated for C₂₁H₂₅F₃N₇O₄S₂ (M+H)⁺: m/z=560.14; Found 560.0. LC-MS calc. for C₂₁H₂₃F₃N₇O₃S₂[MS−OH]⁺:542.1; Found: 542.0.

### Step 2. 2-(1-Hydroxyethyl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile

A solution of 4-chloro-N-[1-(1-methylpyrazol-4-yl)sulfonylpiperidin-4-yl]-5-(trifluoro-methyl)pyrimidin-2-amine (18.0 mg, 30.0 μmol) in 1.00 mL of DMF was added POCl₃ (20.0 μL, 0.190 mmol) in 0.500 mL of DMF at 0° C. and stirred for 1 h. The LCMS and HPLC showed the conversion rate >95%. The reaction was purified by prep-HPLC on C₁₈ column (30×250 mm, 10 m) using mobile phase 28% to 58% MeCN/H₂O (w/0.1% TFA). The desired fractions were collected, concentrated and freeze-dried to give the title compound (12.0 mg, 68.9% yield) as a white solid. LCMS calculated for C₂₁H₂₃F₃N₇O₃S₂ (M+H)⁺: m/z=542.13; Found 542.1. ¹H NMR (300 MHz, DMSO-d₆) δ 8.66 (s, 1H), 8.38-8.19 (m, 2H), 7.78 (d, J=3.2 Hz, 1H), 7.73 (d, J=9.1 Hz, 1H), 5.17 (q, J=6.4 Hz, 1H), 3.91 (s, 3H), 3.86-3.74 (m, 1H), 3.58-3.35 (m, 2H), 2.60-2.37 (m, 2H), 2.06-1.88 (m, 2H), 1.74-1.55 (m, 2H), 1.50 (d, J=6.4 Hz, 3H).

### Example 91

Example 91 shown below in Table 4 was prepared in accordance with the synthetic protocols set forth in Example 90 above using the appropriate intermediates, as well as commercial starting materials.

TABLE 4
Example 91

| Ex. | Molecular Structure and Name | Analytical Data (LCMS and/or NMR) |
|---|---|---|
| 91 | 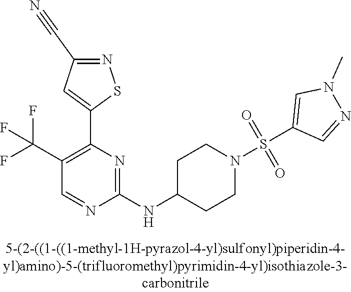 [SMILES: Cn1cc(S(=O)(=O)N2CCC(Nc3ncc(C(F)(F)F)c(-c4cc(C#N)ns4)n3)CC2)cn1] | LCMS calculated for C₁₈H₁₈F₃N₈O₂S₂ (M + H)⁺: m/z = 499.09; Found 499.0. ¹H NMR (300 MHz, DMSO-d₆) δ 8.78 (s, 1H), 8.51 (dd, J = 14.9, 7.4 Hz, 1H), 8.34 (d, J = 3.7 Hz, 1H), 8.30 (d, J = 3.2 Hz, 1H), 7.79 (d, J = 5.3 Hz, 1H), 3.92 (d, J = 2.0 Hz, 3H), 3.89-3.70 (m, 1H), 3.49 (d, J = 11.8 Hz, 2H), 2.61-2.40 (m, 2H), 2.06-1.88 (m, 2H), 1.64 (td, J = 13.9, 3.5 Hz, 2H). |

### Example 92. 2-(2-((1-((1-Methyl-1H-imidazol-4-yl)sulfonyl)piperidin-4-yl)amino)-5-(trifluoro-methyl)pyrimidin-4-yl)thiazole-4-carboxamide

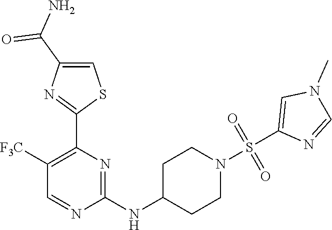 [SMILES: Cn1cnc(S(=O)(=O)N2CCC(Nc3ncc(C(F)(F)F)c(-c4nc(C(N)=O)cs4)n3)CC2)c1]

### Step 1. 2-(2-Chloro-5-(trifluoromethyl)-3,4-dihydropyrimidin-4-yl)thiazole-4-carboxamide

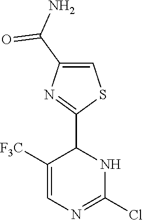 [SMILES: NC(=O)c1csc(C2NC(Cl)=NC=C2C(F)(F)F)n1]

To a solution of 2-bromo-1,3-thiazole-4-carboxamide (100 mg, 0.48 mmol) in THF (3.00 mL) was added n-butyllithium (0.58 mL, 1.45 mmol) dropwise at −78° C. The reaction mixture was stirred at −78° C. for 30 min. Then 2-chloro-5-(trifluoromethyl)pyrimidine (106 mg, 0.58 mmol) was added. The resulting mixture was further stirred at −78° C. for 30 min. LCMS showed the starting material was consumed. The reaction was quenched with saturated NH₄Cl solution (3.00 mL) and extracted with EtOAc (3.00 mL×3). The combined organic layers were dried over Na₂SO₄, filtered, and concentrated to yield the title compound (115 mg, 77.2% yield), which was used for the next reaction without further purification. LCMS calculated for C₉H₇ClF₃N₄₀S (M+H)⁺: m/z=311.00/313.00; Found 311.0/313.0.

### Step 2. 2-(2-Chloro-5-(trifluoromethyl)pyrimidin-4-yl)thiazole-4-carboxamide

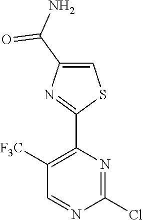 [SMILES: NC(=O)c1csc(-c2nc(Cl)ncc2C(F)(F)F)n1]

To a solution of 2-(2-chloro-5-(trifluoromethyl)-3,4-dihydropyrimidin-4-yl)thiazole-4-carboxamide (115 mg, 0.37 mmol) in THF (1.00 mL) was added 2,3-dichloro-5,6-dicyano-p-benzoquinone (84.0 mg, 0.37 mmol). The reaction mixture was stirred at RT for 1 h. LCMS showed the starting material was consumed. The reaction was diluted with DCM (3.00 mL) and washed with 10% K₂CO₃ solution (3.00 mL) and brine (3.00 mL), dried over Na₂SO₄, filtered and concentrated. The residue was purified by prep-HPLC on a C₁₈ column eluting with MeCN/H₂O (20-60%) with 0.1% TFA to yield the title compound (15.0 mg, 13.1% yield). LCMS calculated for C₉H₅ClF₃N₄₀S (M+H)⁺: m/z=308.98/310.98; Found 309.0/311.0.

### Step 3. Tert-butyl 4-((4-(4-carbamoylthiazol-2-yl)-5-(trifluoromethyl)pyrimidin-2-yl)amino)piperidine-1-carboxylate

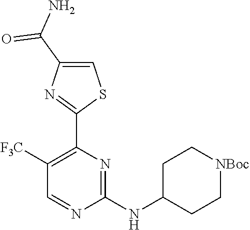 [SMILES: CC(C)(C)OC(=O)N1CCC(Nc2ncc(C(F)(F)F)c(-c3nc(C(N)=O)cs3)n2)CC1]

To a solution of 2-(2-chloro-5-(trifluoromethyl)pyrimidin-4-yl)thiazole-4-carboxamide (15.0 mg, 0.0486 mmol) and 4-amino-1-boc-piperidine (11.7 mg, 0.0583 mmol) in NMP (1.00 mL) was added N,N-diisopropylethylamine (25.4 μL, 0.146 mmol). The reaction mixture was stirred at 110° C. for 1 h. LCMS showed the starting material was consumed. The reaction was diluted with MeOH (2.00 mL) and purified by prep-HPLC on a C₁₈ column eluting with MeCN/H₂O (20-70%) with 0.1% TFA to yield the title compound (20.1 mg, 87.1% yield). LCMS calculated for C₁₉H₂₄F₃N₆O₃S (M+H)⁺: m/z=473.16; Found 473.1.

### Step 4. 2-(2-(Piperidin-4-ylamino)-5-(trifluoromethyl)pyrimidin-4-yl)thiazole-4-carboxamide

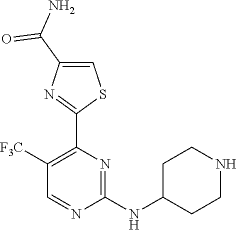 [SMILES: NC(=O)c1csc(-c2nc(NC3CCNCC3)ncc2C(F)(F)F)n1]

To a solution of tert-butyl 4-((4-(4-carbamoylthiazol-2-yl)-5-(trifluoromethyl)pyrimidin-2-yl)amino)piperidine-1-carboxylate (20.0 mg, 0.0423 mmol) in DCM (0.500 mL) was added 2,2,2-trifluoroacetic acid (0.200 mL, 2.61 mmol). The reaction mixture was stirred at RT for 30 min. LCMS showed the starting material was consumed. The solvent was removed under reduced pressure to yield the title compound (16.0 mg, quant.), which was used for the next reaction without further purification. LCMS calculated for C₁₄H₁₆F₃N₆OS (M+H)⁺: m/z=373.11; Found 373.0.

### Step 5. 2-(2-((1-((1-Methyl-1H-imidazol-4-yl)sulfonyl)piperidin-4-yl)amino)-5-(trifluoro-methyl)pyrimidin-4-yl)thiazole-4-carboxamide

To a solution of 2-(2-(piperidin-4-ylamino)-5-(trifluoromethyl)pyrimidin-4-yl)thiazole-4-carboxamide (16.0 mg, 0.043 mmol) and N,N-diisopropylethylamine (23.0 μL, 0.13 mmol) in DMA (1.00 mL) was added 1-methylimidazole-4-sulfonyl chloride (9.30 mg, 0.052 mmol) at 0° C. The reaction mixture was stirred at RT for 30 min. LCMS showed the starting material was consumed. The reaction was diluted with MeOH (2 mL) and purified by prep-HPLC on a C₁₈ column eluting with MeCN/H₂O (20-60%) with 0.1% TFA to yield the title compound (11.2 mg, 50.3% yield). ¹H NMR (300 MHz, DMSO) δ 9.23 (d, J=2.9 Hz, 1H), 8.62 (d, J=3.4 Hz, 1H), 8.23 (dd, J=15.9, 7.4 Hz, 1H), 7.81 (t, J=7.1 Hz, 3H), 7.57 (s, 1H), 3.81 (d, J=6.6 Hz, 1H), 3.70 (d, J=9.0 Hz, 3H), 3.56 (d, J=12.7 Hz, 2H), 2.64 (dd, J=27.5, 17.0 Hz, 2H), 1.90 (t, J=14.1 Hz, 2H), 1.54 (dd, J=22.5, 11.7 Hz, 2H). LCMS calculated for C₁₈H₂₀F₃N₈O₃S₂(M+H)⁺: m/z=517.11; Found 517.0.

### Example 93. 5-(2-((1-((2-Aminothiazol-5-yl)sulfonyl)piperidin-4-yl)amino)-5-(trifluoromethyl) pyrimidin-4-yl)-2-methylthiophene-3-carbonitrile

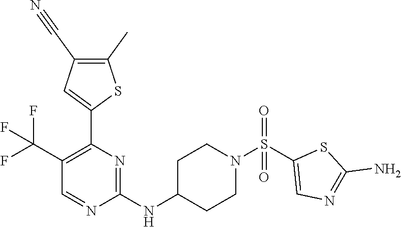 [SMILES: Cc1sc(-c2nc(NC3CCN(S(=O)(=O)c4cnc(N)s4)CC3)ncc2C(F)(F)F)cc1C#N]

### Step 1. N-(5-((4-((4-(4-cyano-5-methylthiophen-2-yl)-5-(trifluoromethyl)pyrimidin-2-yl)amino)piperidin-1-yl)sulfonyl)thiazol-2-yl)acetamide

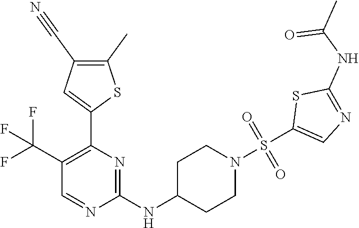 [SMILES: CC(=O)Nc1ncc(S(=O)(=O)N2CCC(Nc3ncc(C(F)(F)F)c(-c4cc(C#N)c(C)s4)n3)CC2)s1]

N-(5-((4-((4-(4-cyano-5-methylthiophen-2-yl)-5-(trifluoromethyl)pyrimidin-2-yl)amino) piperidin-1-yl)sulfonyl)thiazol-2-yl)acetamide was synthesized according to Example 1 with appropriate starting materials and commercial reagents.

### Step 2: 5-(2-((1-((2-aminothiazol-5-yl)sulfonyl)piperidin-4-yl)amino)-5-(trifluoromethyl) pyrimidin-4-yl)-2-methylthiophene-3-carbonitrile

To solution of N-[5-[4-[[4-(4-cyano-5-methylthiophen-2-yl)-5-(trifluoromethyl) pyrimidin-2-yl]amino]piperidin-1-yl]sulfonyl-1,3-thiazol-2-yl]acetamide (35.0 mg, 0.06 mmol) in ethanol (5.00 mL) was added 4 N HCl (3.00 mL). The reaction was stirred at 90° C. for 6 h. LCMS showed that the starting material was consumed. The solvent was then removed under reduced pressure. The crude was purified by prep-HPLC on C₁₈ column (30×250 mm, 10 m) using mobile phase 10-60% MeCN/H₂O to afford the title compound (20 mg, 62.9% yield). ¹H NMR (300 MHz, DMSO) δ 8.66 (d, J=6.2 Hz, 1H), 8.29 (dd, J=25.0, 7.4 Hz, 1H), 7.96 (s, 2H), 7.73 (d, J=9.7 Hz, 1H), 7.46 (d, J=3.9 Hz, 1H), 3.85 (s, 1H), 3.51 (s, 2H), 2.79-2.54 (m, 5H), 1.96 (d, J=15.0 Hz, 2H), 1.72-1.56 (m, 2H). LCMS calc. for C₁₉H₁₉F₃N₇O₂S₃ [M+H]⁺: m/z=530.1; Found 530.0.

### Example 94

Example 94 shown below in Table 5 was prepared in accordance with the synthetic protocols set forth in Example 93 above using the appropriate intermediates, as well as commercial starting materials.

TABLE 5
Example 94

| Ex. | Molecular Structure and Name | Analytical Data (LCMS and/or NMR) |
|---|---|---|
| 94 | 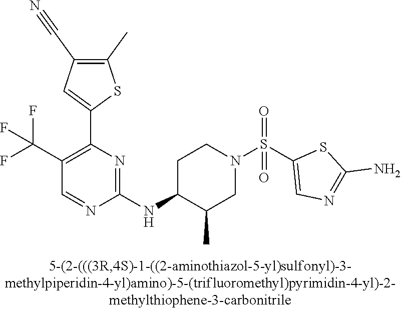 [SMILES: Cc1sc(-c2nc(N[C@H]3CCN(S(=O)(=O)c4cnc(N)s4)C[C@H]3C)ncc2C(F)(F)F)cc1C#N] | LCMS calc. for C₂₀H₂₁F₃N₇O₂S₃ [M + H]⁺: m/z = 544.1; Found 544.0. ¹H NMR (300 MHz, DMSO) δ 8.66 (d, J = 9.3 Hz, 1H), 8.35 (dd, J = 34.7, 8.2 Hz, 1H), 7.94 (s, 2H), 7.72 (d, J = 12.1 Hz, 1H), 7.45 (s, 1H), 4.22-4.00 (m, 1H), 3.20-2.88 (m, 4H), 2.70 (d, J = 15.3 Hz, 3H), 2.27-2.13 (m, 1H), 1.91-1.72 (m, 2H), 0.89 (d, J = 6.9 Hz, 3H). |

Examples 95-106 shown below in Table 6 were prepared in accordance with the synthetic protocols set forth in Examples 1-94 and 107-109 using the appropriate intermediates, as well as commercial starting materials.

TABLE 6
Examples 95-106

| Ex. | Molecular Structure and Name | Analytical Data (LCMS and/or NMR) |
|---|---|---|
| 95 | 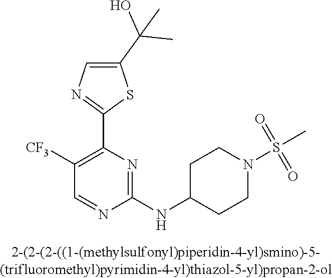 [SMILES: CC(C)(O)c1cnc(-c2nc(NC3CCN(S(C)(=O)=O)CC3)ncc2C(F)(F)F)s1] | LCMS calc. for C₁₇H₂₃F₃N₅O₃S₂ [M + H]⁺: m/z = 466.1; Found 466.1. |
| 96 | 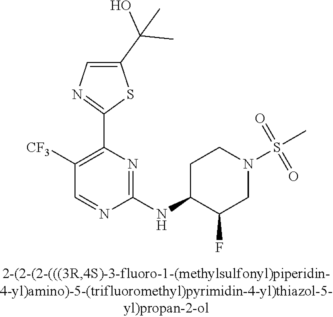 [SMILES: CC(C)(O)c1cnc(-c2nc(N[C@H]3CCN(S(C)(=O)=O)C[C@H]3F)ncc2C(F)(F)F)s1] | LCMS calc. for C₁₇H₂₂F₄N₅O₃S₂ [M + H]⁺: m/z = 484.1; Found 484.1. |
| 97 | 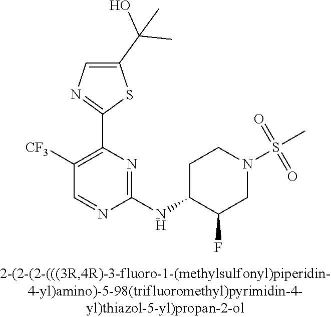 [SMILES: CC(C)(O)c1cnc(-c2nc(N[C@@H]3CCN(S(C)(=O)=O)C[C@H]3F)ncc2C(F)(F)F)s1] | LCMS calc. for C₁₇H₂₂F₄N₅O₃S₂ [M + H]⁺: m/z = 484.1; Found 484.2. |
| 98 | 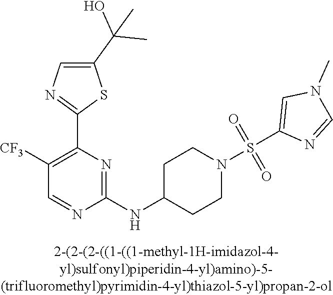 [SMILES: Cn1cnc(S(=O)(=O)N2CCC(Nc3ncc(C(F)(F)F)c(-c4ncc(C(C)(C)O)s4)n3)CC2)c1] | LCMS calc. for C₂₀H₂₅F₃N₇O₃S₂ [M + H]⁺: m/z = 532.1; Found 532.1. |
| 99 | 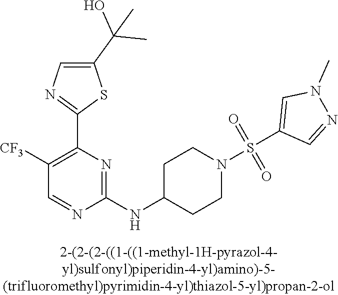 [SMILES: Cn1cc(S(=O)(=O)N2CCC(Nc3ncc(C(F)(F)F)c(-c4ncc(C(C)(C)O)s4)n3)CC2)cn1] | LCMS calc. for C₂₀H₂₅F₃N₇O₃S₂ [M + H]⁺: m/z = 532.1; Found 532.2. |
| 100 | 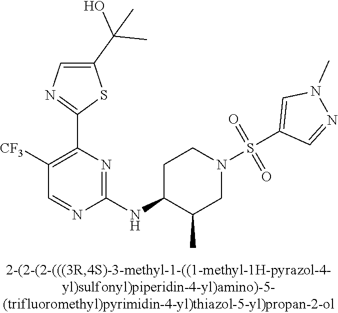 [SMILES: C[C@@H]1CN(S(=O)(=O)c2cnn(C)c2)CC[C@@H]1Nc1ncc(C(F)(F)F)c(-c2ncc(C(C)(C)O)s2)n1] | LCMS calc. for C₂₁H₂₇F₃N₇O₃S₂ [M + H]⁺: m/z = 546.2; Found 546.0. ¹H NMR (300 MHz, DMSO) δ 8.74-8.66 (m, 1H), 8.37-8.14 (m, 2H), 7.93-7.89 (m, 1H), 7.79 (s, 1H), 4.20-4.04 (m, 1H), 3.93 (s, 3H), 3.25-2.70 (m, 4H), 2.27-2.20 (m, 1H), 1.99-1.71 (m, 2H), 1.56 (s, 6H), 0.97-0.86 (m, 3H). |
| 101 | 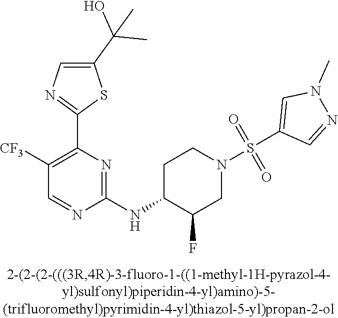 [SMILES: Cn1cc(S(=O)(=O)N2CC[C@@H](Nc3ncc(C(F)(F)F)c(-c4ncc(C(C)(C)O)s4)n3)[C@H](F)C2)cn1] | LCMS calc. for C₂₀H₂₄F₄N₇O₃S₂ [M + H]⁺: m/z = 550.1; Found 550.0. |
| 102 | 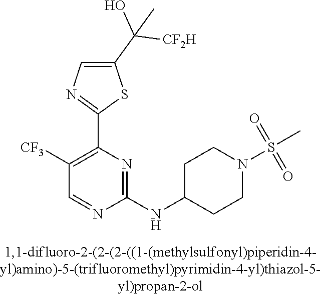 [SMILES: CC(O)(c1cnc(-c2nc(NC3CCN(S(C)(=O)=O)CC3)ncc2C(F)(F)F)s1)C(F)F] | LCMS calc. for C₁₇H₂₁F₅N₅O₃S₂ [M + H]⁺: m/z = 502.1; Found 502.0. |
| 103 | 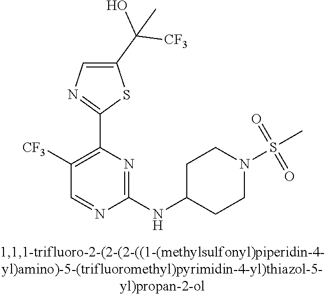 [SMILES: CC(O)(c1cnc(-c2nc(NC3CCN(S(C)(=O)=O)CC3)ncc2C(F)(F)F)s1)C(F)(F)F] | LCMS calc. for C₁₇H₂₀F₆N₅O₃S₂ [M + H]⁺: m/z = 520.1; Found 520.2. |
| 104 | 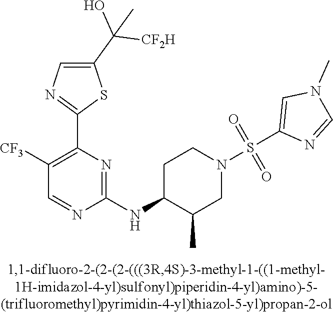 [SMILES: C[C@@H]1CN(S(=O)(=O)c2cn(C)cn2)CC[C@@H]1Nc1ncc(C(F)(F)F)c(-c2ncc(C(C)(O)C(F)F)s2)n1] | LCMS calc. for C₂₁H₂₅F₅N₇O₃S₂ [M + H]⁺: m/z = 582.1; Found 582.2. |
| 105 | 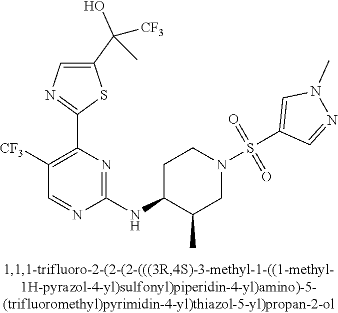 [SMILES: C[C@@H]1CN(S(=O)(=O)c2cnn(C)c2)CC[C@@H]1Nc1ncc(C(F)(F)F)c(-c2ncc(C(C)(O)C(F)(F)F)s2)n1] | LCMS calc. for C₂₁H₂₄F₆N₇O₃S₂ [M + H]⁺: m/z = 600.1; Found 600.2. |
| 106 | 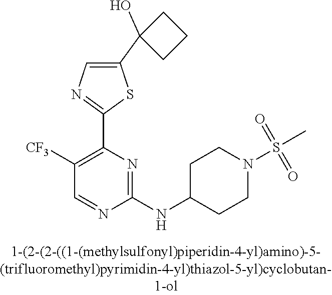 [SMILES: CS(=O)(=O)N1CCC(Nc2ncc(C(F)(F)F)c(-c3ncc(C4(O)CCC4)s3)n2)CC1] | LCMS calc. for C₁₈H₂₃F₃N₅O₃S₂ [M + H]⁺: m/z = 478.1; Found 478.1. |

### Example 107. 2-Methyl-1-(2-(2-((1-(methylsulfonyl)piperidin-4-yl)amino)-5-(trifluoromethyl) pyrimidin-4-yl)thiazol-5-yl)propan-2-ol

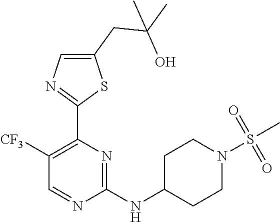 [SMILES: CC(C)(O)Cc1cnc(-c2nc(NC3CCN(S(C)(=O)=O)CC3)ncc2C(F)(F)F)s1]

### Step 1. Tert-butyl 4-((4-(5-(2-hydroxy-2-methylpropyl)thiazol-2-yl)-5-(trifluoromethyl)-pyrimidin-2-yl)amino)piperidine-1-carboxylate

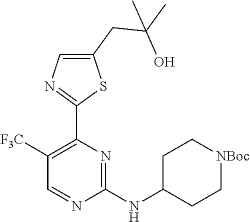 [SMILES: CC(C)(O)Cc1cnc(-c2nc(NC3CCN(C(=O)OC(C)(C)C)CC3)ncc2C(F)(F)F)s1]

To a solution of 1-(2-bromo-1,3-thiazol-5-yl)-2-methylpropan-2-ol (Intermediate 7) (55.8 mg, 0.236 mmol) and tert-butyl 4-[[5-(trifluoromethyl)-4-trimethylstannylpyrimidin-2-yl]amino]piperidine-1-carboxylate (Intermediate 23) (100 mg, 0.196 mmol) in 1,4-dioxane (1.5 mL) was added bis(triphenylphosphine)palladium(II) dichloride (27.6 mg, 0.0393 mmol), copper(I) iodide (7.5 mg, 0.039 mmol) and lithium chloride (25.0 mg, 0.589 mmol). The reaction mixture was bubbled with N₂ for 1 min and stirred at 100° C. for 3 h. LCMS showed the starting material was consumed. The reaction was quenched with KF (aq) solution and stirred at RT for 1 h. The layer was separated, and the aqueous layer was extracted with DCM (3.0 mL×3). The combined organic layers were dried over Na₂SO₄, filtered and concentrated. The residue was purified by prep-HPLC on a C₁₈ column eluting with MeCN/H₂O (20-70%) with 0.1% TFA to yield the title compound (53.2 mg, 54.1% yield). LCMS calculated for C₂₂H₃₁F₃N₅O₃S (M+H)⁺: m/z=502.21; Found 502.0.

### Step 2. 2-Methyl-1-(2-(2-(piperidin-4-ylamino)-5-(trifluoromethyl)pyrimidin-4-yl)thiazol-5-yl)propan-2-ol

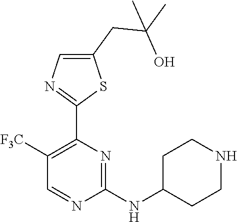 [SMILES: CC(C)(O)Cc1cnc(-c2nc(NC3CCNCC3)ncc2C(F)(F)F)s1]

To a solution of tert-butyl 4-((4-(5-(2-hydroxy-2-methylpropyl)thiazol-2-yl)-5-(trifluoro-methyl)pyrimidin-2-yl)amino)piperidine-1-carboxylate (27.1 mg, 0.0538 mmol) in DCM (0.50 mL) was added 2,2,2-trifluoroacetic acid (0.20 mL). The reaction mixture was stirred at RT for 1 h. LCMS showed the starting material was consumed. The solvent was removed under reduced pressure to yield the title compound (22.0 mg, quant.), which was used for the next reaction without further purification. LCMS calculated for C₁₇H₂₃F₃N₅OS (M+H)⁺: m/z=402.16; Found 402.1.

### Step 3. 2-Methyl-1-(2-(2-((1-(methylsulfonyl)piperidin-4-yl)amino)-5-(trifluoromethyl) pyrimidin-4-yl)thiazol-5-yl)propan-2-ol

To a solution of 2-methyl-1-(2-(2-(piperidin-4-ylamino)-5-(trifluoromethyl)pyrimidin-4-yl)thiazol-5-yl)propan-2-ol (16.0 mg, 0.040 mmol) and N,N-diisopropylethylamine (20.9 μL, 0.12 mmol) in DMA (1.00 mL) was added methanesulfonyl chloride (5.5 mg, 0.048 mmol). The reaction mixture was stirred at RT for 20 min. LCMS showed the starting material was consumed. The reaction was diluted with MeOH (2.0 mL) and purified by prep-HPLC on a C₁₈ column eluting with MeCN/H₂O (20-60%) with 0.1% TFA to yield the title compound (9.2 mg, 46% yield). LCMS calc. for C₁₈H₂₅F₃N₅O₃S₂ [M+H]⁺: m/z=480.14; Found 480.3. ¹H NMR (300 MHz, DMSO) δ 8.76-8.66 (m, 1H), 8.30-8.19 (m, 1H), 7.86-7.75 (m, 1H), 4.72 (s, 1H), 4.05-3.70 (m, 1H), 3.63-3.50 (m, 2H), 3.01-2.76 (m, 7H), 2.10-1.81 (m, 2H), 1.72-1.52 (m, 2H), 1.13 (s, 6H).

### Example 108. 1-(2-(2-(((3R,4S)-3-fluoro-1-(methylsulfonyl)piperidin-4-yl)amino)-5-(trifluoromethyl)pyrimidin-4-yl)thiazol-5-yl)-2-methylpropan-2-ol

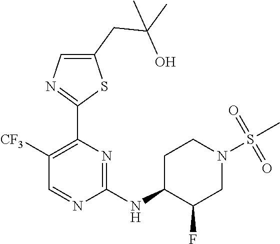 [SMILES: CC(C)(O)Cc1cnc(-c2nc(N[C@H]3CCN(S(C)(=O)=O)C[C@H]3F)ncc2C(F)(F)F)s1]

### Step 1. Tert-butyl (3R,4S)-3-fluoro-4-((4-(5-(2-hydroxy-2-methylpropyl)thiazol-2-yl)-5-(trifluoromethyl)pyrimidin-2-yl)amino)piperidine-1-carboxylate

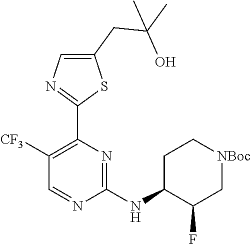 [SMILES: CC(C)(O)Cc1cnc(-c2nc(N[C@H]3CCN(C(=O)OC(C)(C)C)C[C@H]3F)ncc2C(F)(F)F)s1]

To a solution of 1-(2-bromo-1,3-thiazol-5-yl)-2-methylpropan-2-ol (Intermediate 7) (178 mg, 0.752 mmol) and tert-butyl (3R,4S)-3-fluoro-4-((5-(trifluoromethyl)-4-(trimethylstannyl)-pyrimidin-2-yl)amino)piperidine-1-carboxylate (Intermediate 30) (396 mg, 0.752 mmol) in 1,4-dioxane (3.76 mL) was added bis(triphenylphosphine)palladium(II) dichloride (106 mg, 0.15 mmol), copper(I) iodide (28.6 mg, 0.15 mmol) and lithium chloride (95.7 mg, 2.26 mmol). The reaction mixture was bubbled with N₂ for 1 min and stirred at 100° C. for 3 h. LCMS showed the starting material was consumed. The reaction was quenched with KF (aq) solution and stirred at RT for 1 h. The layer was separated, and the aqueous layer was extracted with DCM (10.0 mL×3). The combined organic layers were dried over Na₂SO₄, filtered and concentrated. The residue was purified by prep-HPLC on a C₁₈ column eluting with MeCN/H₂O (15-75%) with 0.1% TFA to yield the title compound (60.2 mg, 15.3% yield). LCMS calculated for C₂₂H₃₀F₄N₅O₃S (M+H)⁺: m/z=520.20; Found 520.1.

### Step 2. 1-(2-(2-(((3R,4S)-3-fluoropiperidin-4-yl)amino)-5-(trifluoromethyl)pyrimidin-4-yl)thiazol-5-yl)-2-methylpropan-2-ol

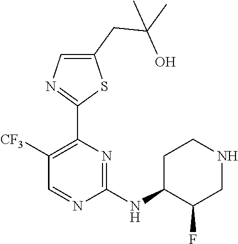 [SMILES: CC(C)(O)Cc1cnc(-c2nc(N[C@H]3CCNC[C@H]3F)ncc2C(F)(F)F)s1]

To a solution of tert-butyl 4-((4-(5-(2-hydroxy-2-methylpropyl)thiazol-2-yl)-5-(trifluoro-methyl)pyrimidin-2-yl)amino)piperidine-1-carboxylate (57.4 mg, 0.11 mmol) in DCM (0.50 mL) was added 2,2,2-trifluoroacetic acid (0.20 mL). The reaction mixture was stirred at RT for 1 h. LCMS showed the starting material was consumed. The solvent was removed under reduced pressure to yield the title compound (71 mg, quant.), which was used for the next reaction without further purification. LCMS calculated for C₁₇H₂₂F₄N₅OS (M+H)⁺: m/z=420.15; Found 420.6.

### Step 3. 1-(2-(2-(((3R,4S)-3-fluoro-1-(methylsulfonyl)piperidin-4-yl)amino)-5-(trifluoro-methyl)pyrimidin-4-yl)thiazol-5-yl)-2-methylpropan-2-ol

To a solution of 1-(2-(2-(((3R,4S)-3-fluoropiperidin-4-yl)amino)-5-(trifluoromethyl)pyrimidin-4-yl)thiazol-5-yl)-2-methylpropan-2-ol (16.0 mg, 0.038 mmol) and N,N-diisopropylethylamine (20.9 μL, 0.12 mmol) in DMA (1.00 mL) was added methanesulfonyl chloride (5.2 mg, 0.046 mmol). The reaction mixture was stirred at RT for 20 min. LCMS showed the starting material was consumed. The reaction was diluted with MeOH (2.0 mL) and purified by prep-HPLC on a C₁₈ column eluting with MeCN/H₂O (20-60%) with 0.1% TFA to yield the title compound (14.3 mg, 75% yield). LCMS calc. for C₁₈H₂₄F₄N₅O₃S₂ [M+H]⁺: m/z=498.13; Found 498.1. ¹H NMR (300 MHz, DMSO) δ 8.75 (s, 1H), 8.49-8.35 (m, 1H), 7.86-7.79 (m, 1H), 5.12-4.81 (m, 1H), 4.73 (s, 1H), 4.31-3.98 (m, 1H), 3.90-3.55 (m, 2H), 3.26-2.81 (m, 7H), 2.10-1.72 (m, 2H), 1.13 (s, 6H).

### Example 109. 1-(2-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiazol-5-yl)-2-methylpropan-2-ol

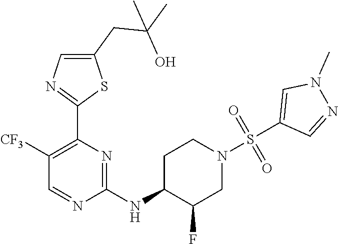 [SMILES: Cn1cc(S(=O)(=O)N2CC[C@H](Nc3ncc(C(F)(F)F)c(-c4ncc(CC(C)(C)O)s4)n3)[C@H](F)C2)cn1]

To a solution of 1-(2-(2-(((3R,4S)-3-fluoropiperidin-4-yl)amino)-5-(trifluoromethyl) pyrimidin-4-yl)thiazol-5-yl)-2-methylpropan-2-ol (Example 108, Step 2) (15.0 mg, 0.036 mmol) and N,N-diisopropylethylamine (20.9 μL, 0.12 mmol) in DMA (1.00 mL) was added 1-methylpyrazole-4-sulfonyl chloride (7.8 mg, 0.043 mmol). The reaction mixture was stirred at RT for 20 min LCMS showed the starting material was consumed. The reaction was diluted with MeOH (2.0 mL) and purified by prep-HPLC on a C₁₈ column eluting with MeCN/H₂O (20-60%) with 0.1% TFA to yield the title compound (17.5 mg, 86% yield). LCMS calc. for C₂₁H₂₆F₄N₇O₃S₂ [M+H]⁺: m/z 564.15; Found 564.0. ¹H NMR (300 MHz, DMSO) δ 8.76-8.66 (m, 1H), 8.43-8.31 (m, 2H), 7.86-7.76 (m, 2H), 5.11-4.86 (m, 1H), 4.76-4.65 (m, 1H), 4.25-3.74 (m, 5H), 3.67-3.58 (m, 1H), 3.01-2.92 (m, 2H), 2.78-2.49 (m, 2H), 2.12-1.67 (m, 2H), 1.13 (s, 6H).

### Examples 110-155

Examples 110-155 shown below in Table 7 were prepared in accordance with the synthetic protocols set forth in Examples 1-94 and 107-109 using the appropriate intermediates, as well as commercial starting materials.

TABLE 7
Examples 110-155

| Ex. | Molecular Structure and Name | Analytical Data (LCMS and/or NMR) |
|---|---|---|
| 110 | 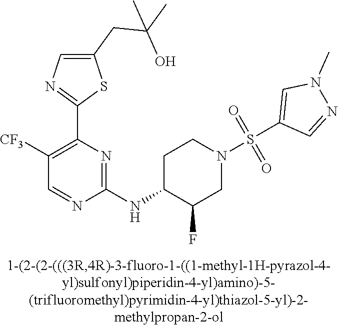 [SMILES: Cn1cc(S(=O)(=O)N2CC[C@@H](Nc3ncc(C(F)(F)F)c(-c4ncc(CC(C)(C)O)s4)n3)[C@H](F)C2)cn1] | LCMS calc. for C₂₁H₂₆F₄N₇O₃S₂ [M + H]⁺: m/z = 564.1; Found 564.2. |
| 111 | 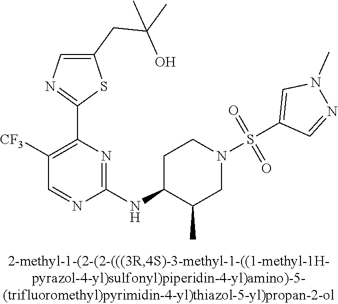 [SMILES: C[C@@H]1CN(S(=O)(=O)c2cnn(C)c2)CC[C@@H]1Nc1ncc(C(F)(F)F)c(-c2ncc(CC(C)(C)O)s2)n1] | LCMS calc. for C₂₂H₂₉F₃N₇O₃S₂ [M + H]⁺: m/z = 560.1; Found 560.3. ¹H NMR (300 MHz, DMSO) δ 8.75-8.62 (m, 1H), 8.41-8.21 (m, 2H), 7.84-7.75 (m, 2H), 5.11-4.86 (m, 1H), 4.18-4.01 (m, 1H), 3.98-3.84 (m, 3H), 3.23-2.72 (m, 6H), 2.26-2.12 (m, 1H), 1.96-1.62 (m, 2H), 1.12 (s, 6H), 0.98-0.83 (m, 3H). |
| 112 | 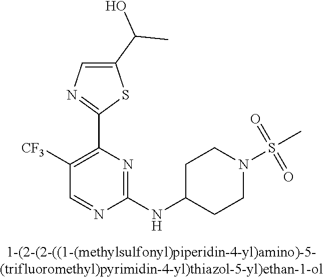 [SMILES: CC(O)c1cnc(-c2nc(NC3CCN(S(C)(=O)=O)CC3)ncc2C(F)(F)F)s1] | LCMS calc. for C₁₆H₂₁F₃N₅O₃S₂ [M + H]⁺: m/z = 452.1; Found 452.1. |
| 113 | 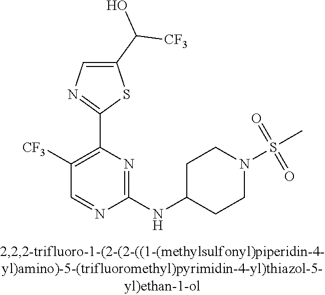 [SMILES: CS(=O)(=O)N1CCC(Nc2ncc(C(F)(F)F)c(-c3ncc(C(O)C(F)(F)F)s3)n2)CC1] | LCMS calc. for C₁₆H₁₈F₆N₅O₃S₂ [M + H]⁺: m/z = 506.1; Found 506.1. |
| 114 | 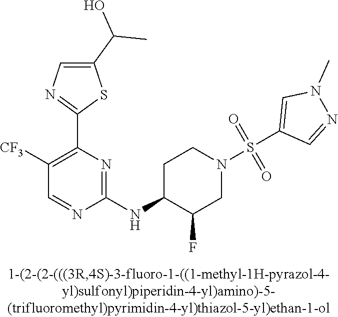 [SMILES: CC(O)c1cnc(-c2nc(N[C@H]3CCN(S(=O)(=O)c4cnn(C)c4)C[C@H]3F)ncc2C(F)(F)F)s1] | LCMS calc. for C₁₉H₂₂F₄N₇O₃S₂ [M + H]⁺: m/z = 536.1; Found 536.1. |
| 115 | 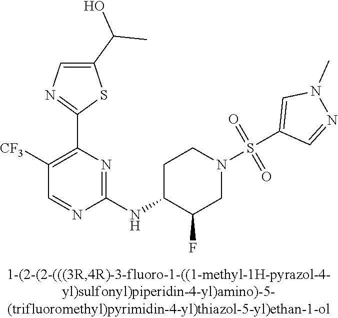 [SMILES: CC(O)c1cnc(-c2nc(N[C@@H]3CCN(S(=O)(=O)c4cnn(C)c4)C[C@H]3F)ncc2C(F)(F)F)s1] | LCMS calc. for C₁₉H₂₂F₄N₇O₃S₂ [M + H]⁺: m/z = 536.1; Found 536.1. |
| 116 | 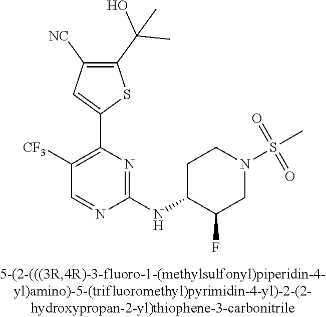 [SMILES: CC(C)(O)c1sc(-c2nc(N[C@@H]3CCN(S(C)(=O)=O)C[C@H]3F)ncc2C(F)(F)F)cc1C#N] | LCMS calc. for C₁₉H₂₂F₄N₅O₃S₂ [M + H]⁺: m/z = 508.1; Found 508.0. |
| 117 | 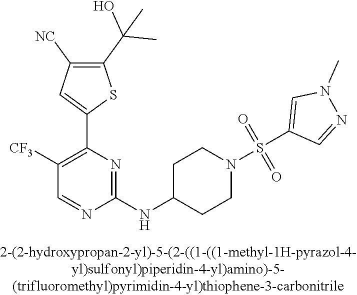 [SMILES: Cn1cc(S(=O)(=O)N2CCC(Nc3ncc(C(F)(F)F)c(-c4cc(C#N)c(C(C)(C)O)s4)n3)CC2)cn1] | LCMS calc. for C₂₂H₂₅F₃N₇O₃S₂ [M + H]⁺: m/z = 556.1; Found 556.2. |
| 118 | 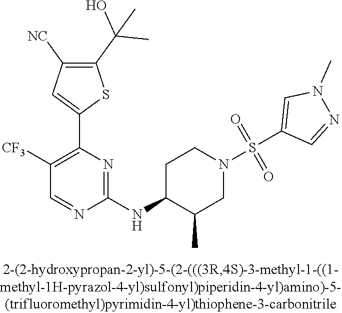 [SMILES: C[C@@H]1CN(S(=O)(=O)c2cnn(C)c2)CC[C@@H]1Nc1ncc(C(F)(F)F)c(-c2cc(C#N)c(C(C)(C)O)s2)n1] | LCMS calc. for C₂₃H₂₇F₃N₇O₃S₂ [M + H]⁺: m/z = 570.1; Found 570.1. |
| 119 | 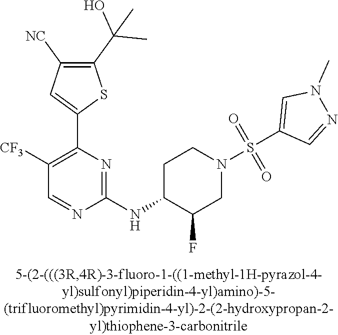 [SMILES: Cn1cc(S(=O)(=O)N2CC[C@@H](Nc3ncc(C(F)(F)F)c(-c4cc(C#N)c(C(C)(C)O)s4)n3)[C@H](F)C2)cn1] | LCMS calc. for C₂₂H₂₄F₄N₇O₃S₂ [M + H]⁺: m/z = 574.1; Found 574.1. |
| 120 | 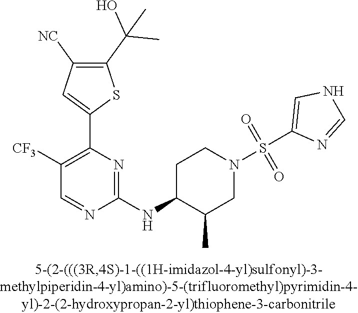 [SMILES: C[C@@H]1CN(S(=O)(=O)c2c[nH]cn2)CC[C@@H]1Nc1ncc(C(F)(F)F)c(-c2cc(C#N)c(C(C)(C)O)s2)n1] | LCMS calc. for C₂₂H₂₅F₃N₇O₃S₂ [M + H]⁺: m/z = 556.1; Found 556.0. |
| 121 | 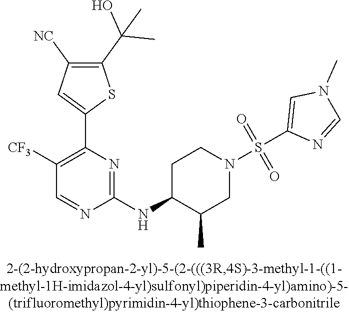 [SMILES: C[C@@H]1CN(S(=O)(=O)c2cn(C)cn2)CC[C@@H]1Nc1ncc(C(F)(F)F)c(-c2cc(C#N)c(C(C)(C)O)s2)n1] | LCMS calc. for C₂₃H₂₇F₃N₇O₃S₂ [M + H]⁺: m/z = 570.1; Found 570.1. |
| 122 | 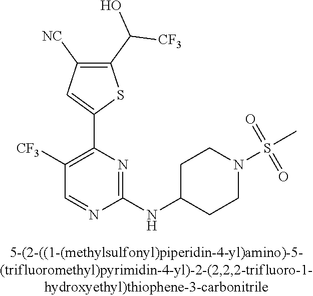 [SMILES: CS(=O)(=O)N1CCC(Nc2ncc(C(F)(F)F)c(-c3cc(C#N)c(C(O)C(F)(F)F)s3)n2)CC1] | LCMS calc. for C₁₈H₁₈F₆N₅O₃S₂ [M + H]⁺: m/z = 530.1; Found 530.0. |
| 123 | 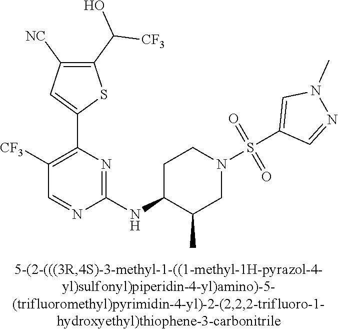 [SMILES: C[C@@H]1CN(S(=O)(=O)c2cnn(C)c2)CC[C@@H]1Nc1ncc(C(F)(F)F)c(-c2cc(C#N)c(C(O)C(F)(F)F)s2)n1] | LCMS calc. for C₂₂H₂₂F₆N₇O₃S₂ [M + H]⁺: m/z = 610.1; Found 610.1. |
| 124 | 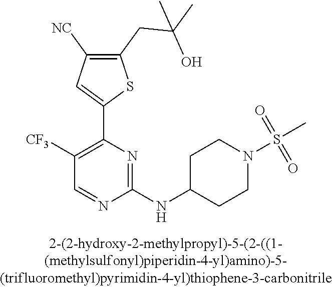 [SMILES: CC(C)(O)Cc1sc(-c2nc(NC3CCN(S(C)(=O)=O)CC3)ncc2C(F)(F)F)cc1C#N] | LCMS calc. for C₂₀H₂₅F₃N₅O₃S₂ [M + H]⁺: m/z = 504.1; Found 504.1. |
| 125 | 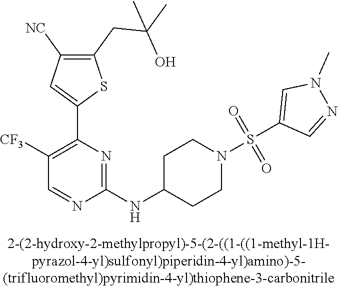 [SMILES: Cn1cc(S(=O)(=O)N2CCC(Nc3ncc(C(F)(F)F)c(-c4cc(C#N)c(CC(C)(C)O)s4)n3)CC2)cn1] | LCMS calc. for C₂₃H₂₇F₃N₇O₃S₂ [M + H]⁺: m/z = 570.1; Found 570.1. |
| 126 | 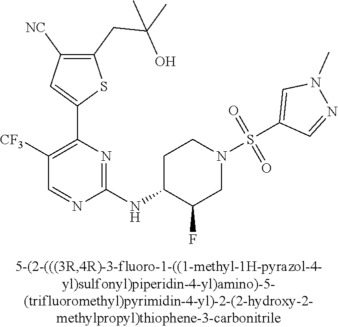 [SMILES: Cn1cc(S(=O)(=O)N2CC[C@@H](Nc3ncc(C(F)(F)F)c(-c4cc(C#N)c(CC(C)(C)O)s4)n3)[C@H](F)C2)cn1] | LCMS calc. for C₂₃H₂₆F₄N₇O₃S₂ [M + H]⁺: m/z = 588.1; Found 588.2. |
| 127 | 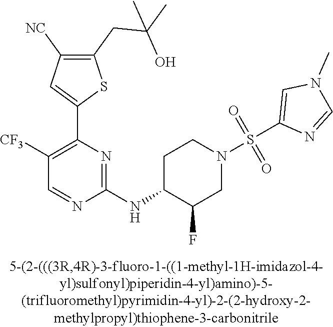 [SMILES: Cn1cnc(S(=O)(=O)N2CC[C@@H](Nc3ncc(C(F)(F)F)c(-c4cc(C#N)c(CC(C)(C)O)s4)n3)[C@H](F)C2)c1] | LCMS calc. for C₂₃H₂₆F₄N₇O₃S₂ [M + H]⁺: m/z = 588.1; Found 588.2. |
| 128 | 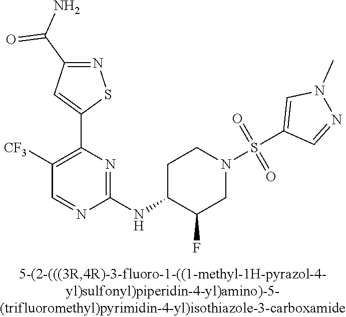 [SMILES: Cn1cc(S(=O)(=O)N2CC[C@@H](Nc3ncc(C(F)(F)F)c(-c4cc(C(N)=O)ns4)n3)[C@H](F)C2)cn1] | LCMS calc. for C₁₈H₁₉F₄N₈O₃S₂ [M + H]⁺: m/z = 535.1; Found 535.1. |
| 129 | 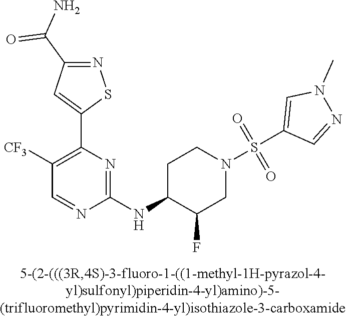 [SMILES: Cn1cc(S(=O)(=O)N2CC[C@H](Nc3ncc(C(F)(F)F)c(-c4cc(C(N)=O)ns4)n3)[C@H](F)C2)cn1] | LCMS calc. for C₁₈H₁₉F₄N₈O₃S₂ [M + H]⁺: m/z = 535.1; Found 535.0. |
| 130 | 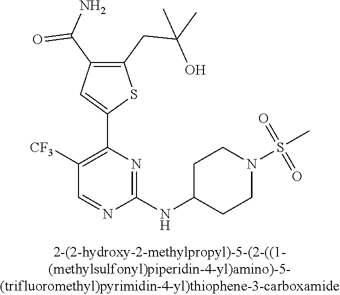 [SMILES: CC(C)(O)Cc1sc(-c2nc(NC3CCN(S(C)(=O)=O)CC3)ncc2C(F)(F)F)cc1C(N)=O] | LCMS calc. for C₂₀H₂₇F₃N₅O₄S₂ [M + H]⁺: m/z = 522.1; Found 522.0. |
| 131 | 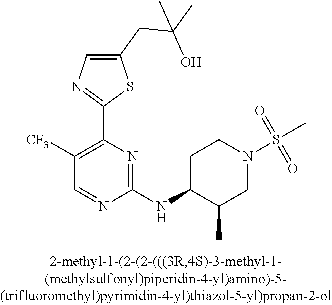 [SMILES: C[C@@H]1CN(S(C)(=O)=O)CC[C@@H]1Nc1ncc(C(F)(F)F)c(-c2ncc(CC(C)(C)O)s2)n1] | LCMS calc. for C₁₉H₂₇F₃N₅O₃S₂ [M + H]⁺: m/z = 494.1; Found 493.9. ¹H NMR (300 MHz, DMSO) δ 8.75-8.67 (m, 1H), 8.34-8.19 (m, 1H), 7.86-7.76 (m, 1H), 4.24 (s, 1H), 3.41-3.01 (m, 4H), 2.96 (s, 2H), 2.88 (s, 3H), 2.25-2.10 (m, 1H), 1.88-1.72 (m, 2H), 1.13 (s, 6H), 0.95-0.78 (m, 3H). |
| 132 | 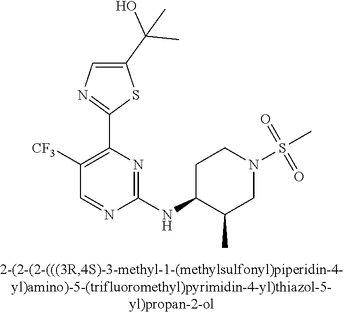 [SMILES: C[C@@H]1CN(S(C)(=O)=O)CC[C@@H]1Nc1ncc(C(F)(F)F)c(-c2ncc(C(C)(C)O)s2)n1] | LCMS calc. for C₁₈H₂₅F₃N₅O₃S₂ [M + H]⁺: m/z = 480.1; Found 480.0. ¹H NMR (300 MHz, DMSO) δ 8.76-8.68 (m, 1H), 8.38-8.16 (m, 1H), 7.93-7.89 (m, 1H), 4.28-4.24 (m, 1H), 3.30-3.01 (m, 4H), 2.88 (s, 3H), 2.26-2.12 (m, 1H), 1.90-1.68 (m, 2H), 1.57 (s, 6H), 0.95-0.85 (m, 3H). |
| 133 | 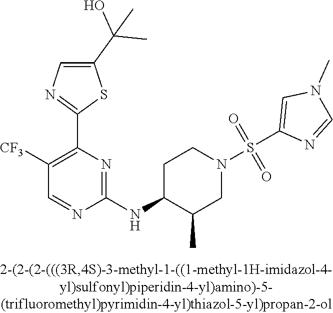 [SMILES: C[C@@H]1CN(S(=O)(=O)c2cn(C)cn2)CC[C@@H]1Nc1ncc(C(F)(F)F)c(-c2ncc(C(C)(C)O)s2)n1] | LCMS calc. for C₂₁H₂₇F₃N₇O₃S₂ [M + H]⁺: m/z = 546.16; Found 546.1. |
| 134 | 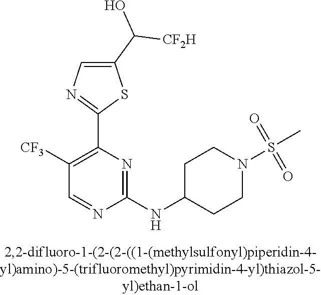 [SMILES: CS(=O)(=O)N1CCC(Nc2ncc(C(F)(F)F)c(-c3ncc(C(O)C(F)F)s3)n2)CC1] | LCMS calc. for C₁₆H₁₉F₅N₅O₃S₂ [M + H]⁺: m/z = 488.08; Found 487.8. |
| 135 | 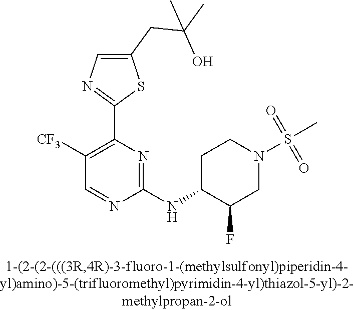 [SMILES: CC(C)(O)Cc1cnc(-c2nc(N[C@@H]3CCN(S(C)(=O)=O)C[C@H]3F)ncc2C(F)(F)F)s1] | LCMS calc. for C₁₈H₂₄F₄N₅O₃S₂ [M + H]⁺: m/z = 498.13; Found 498.0. |
| 136 | 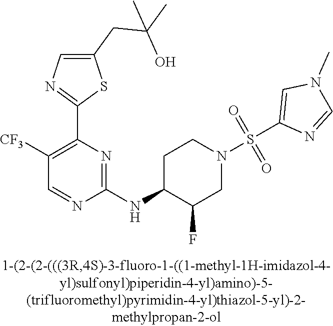 [SMILES: Cn1cnc(S(=O)(=O)N2CC[C@H](Nc3ncc(C(F)(F)F)c(-c4ncc(CC(C)(C)O)s4)n3)[C@H](F)C2)c1] | LCMS calc. for C₂₁H₂₆F₄N₇O₃S₂ [M + H]⁺: m/z = 564.15; Found 564.0. |
| 137 | 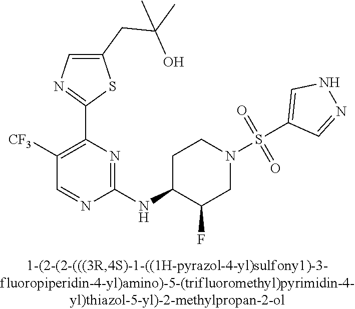 [SMILES: CC(C)(O)Cc1cnc(-c2nc(N[C@H]3CCN(S(=O)(=O)c4cn[nH]c4)C[C@H]3F)ncc2C(F)(F)F)s1] | LCMS calc. for C₂₀H₂₄F₄N₇O₃S₂ [M + H]⁺: m/z = 550.13; Found 550.0. |
| 138 | 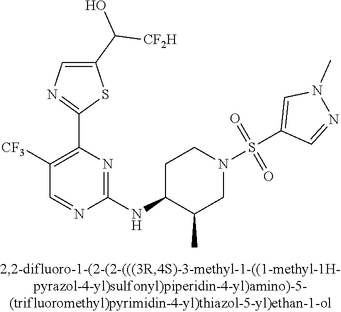 [SMILES: C[C@@H]1CN(S(=O)(=O)c2cnn(C)c2)CC[C@@H]1Nc1ncc(C(F)(F)F)c(-c2ncc(C(O)C(F)F)s2)n1] | LCMS calc. for C₂₀H₂₃F₅N₇O₃S₂ [M + H]⁺: m/z = 568.12; Found 568.0. |
| 139 | 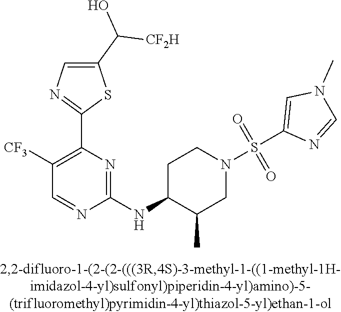 [SMILES: C[C@@H]1CN(S(=O)(=O)c2cn(C)cn2)CC[C@@H]1Nc1ncc(C(F)(F)F)c(-c2ncc(C(O)C(F)F)s2)n1] | LCMS calc. for C₂₀H₂₃F₅N₇O₃S₂ [M + H]⁺: m/z = 568.12; Found 568.0. |
| 140 | 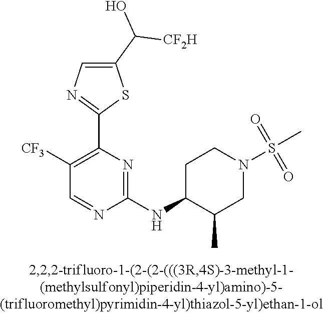 | LCMS calc. for C₁₇H₂₀F₆N₅O₃S₂ [M + H]⁺: m/z = 520.09; Found 520.0. |
| 141 | 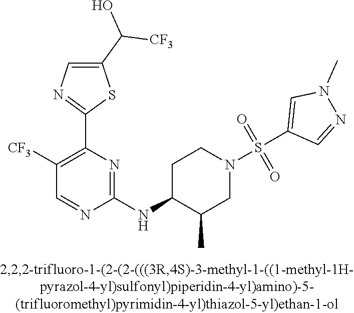 [SMILES: C[C@@H]1CN(S(=O)(=O)c2cnn(C)c2)CC[C@@H]1Nc1ncc(C(F)(F)F)c(-c2ncc(C(O)C(F)(F)F)s2)n1] | LCMS calc. for C₂₀H₂₂F₆N₇O₃S₂ [M + H]⁺: m/z = 586.11; Found 585.8. |
| 142 | 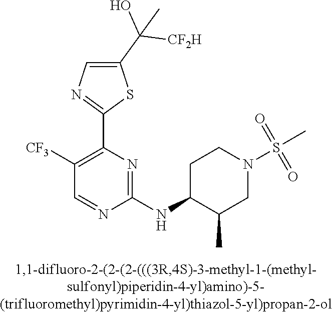 [SMILES: C[C@@H]1CN(S(C)(=O)=O)CC[C@@H]1Nc1ncc(C(F)(F)F)c(-c2ncc(C(C)(O)C(F)F)s2)n1] | LCMS calc. for C₁₈H₂₃F₅N₅O₃S₂ [M + H]⁺: m/z = 516.12; Found 515.9. |
| 143 | 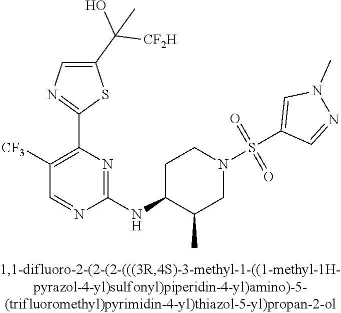 [SMILES: C[C@@H]1CN(S(=O)(=O)c2cnn(C)c2)CC[C@@H]1Nc1ncc(C(F)(F)F)c(-c2ncc(C(C)(O)C(F)F)s2)n1] | LCMS calc. for C₂₁H₂₅F₅N₇O₃S₂ [M + H]⁺: m/z = 582.14; Found 581.9. |
| 144 | 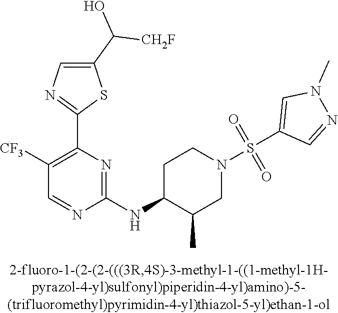 | LCMS calc. for C₂₀H₂₄F₄N₇O₃S₂ [M + H]⁺: m/z = 550.13; Found 550.0. |
| 145 | 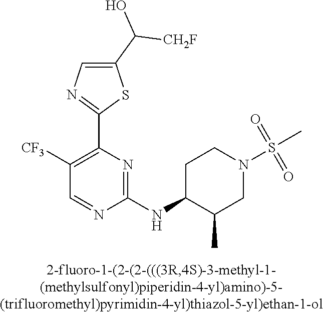 | LCMS calc. for C₁₇H₂₂F₄N₅O₃S₂ [M + H]⁺: m/z = 484.11; Found 483.8. |
| 146 | 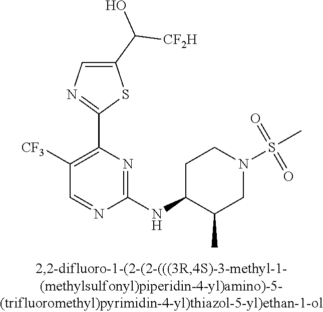 [SMILES: C[C@@H]1CN(S(C)(=O)=O)CC[C@@H]1Nc1ncc(C(F)(F)F)c(-c2ncc(C(O)C(F)F)s2)n1] | LCMS calc. for C₁₇H₂₁F₅N₅O₃S₂ [M + H]⁺: m/z = 502.10; Found 501.8. |
| 147 | 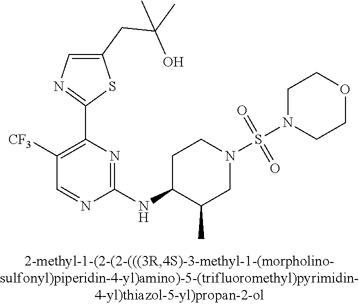 [SMILES: C[C@@H]1CN(S(=O)(=O)N2CCOCC2)CC[C@@H]1Nc1ncc(C(F)(F)F)c(-c2ncc(CC(C)(C)O)s2)n1] | LCMS calc. for C₂₂H₃₂F₃N₆O₄S₂ [M + H]⁺: m/z = 565.19; Found 565.0. |
| 148 | 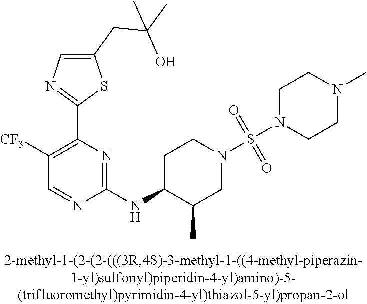 [SMILES: C[C@@H]1CN(S(=O)(=O)N2CCN(C)CC2)CC[C@@H]1Nc1ncc(C(F)(F)F)c(-c2ncc(CC(C)(C)O)s2)n1] | LCMS calc. for C₂₃H₃₅F₃N₇O₃S₂ [M + H]⁺: m/z = 578.22; Found 578.0. |
| 149 | 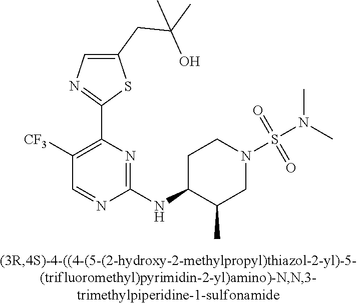 [SMILES: C[C@@H]1CN(S(=O)(=O)N(C)C)CC[C@@H]1Nc1ncc(C(F)(F)F)c(-c2ncc(CC(C)(C)O)s2)n1] | LCMS calc. for C₂₀H₃₀F₃N₆O₃S₂ [M + H]⁺: m/z = 523.18; Found 523.1. |
| 150 | 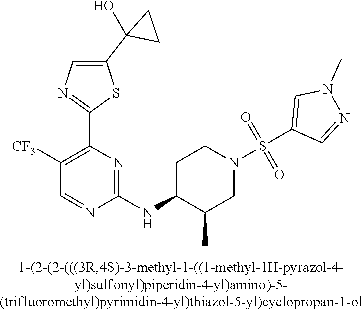 [SMILES: C[C@@H]1CN(S(=O)(=O)c2cnn(C)c2)CC[C@@H]1Nc1ncc(C(F)(F)F)c(-c2ncc(C3(O)CC3)s2)n1] | LCMS calc. for C₂₁H₂₅F₃N₇O₃S₂ [M + H]⁺: m/z = 544.14; Found 544.2. |
| 151 | 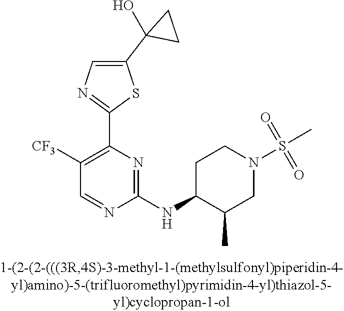 [SMILES: C[C@@H]1CN(S(C)(=O)=O)CC[C@@H]1Nc1ncc(C(F)(F)F)c(-c2ncc(C3(O)CC3)s2)n1] | LCMS calc. for C₁₈H₂₃F₃N₅O₃S₂ [M + H]⁺: m/z = 478.12; Found 478.3. |
| 152 | 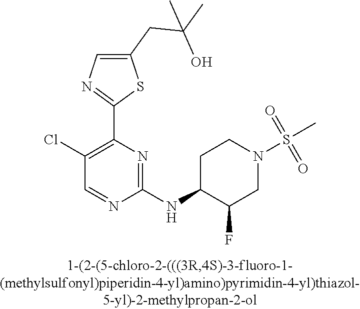 [SMILES: CC(C)(O)Cc1cnc(-c2nc(N[C@H]3CCN(S(C)(=O)=O)C[C@H]3F)ncc2Cl)s1] | LCMS calc. for C₁₇H₂₄ClFN₅O₃S₂ [M + H]⁺: m/z = 464.10; Found 464.4. |
| 153 | 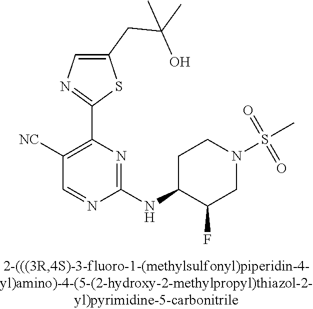 [SMILES: CC(C)(O)Cc1cnc(-c2nc(N[C@H]3CCN(S(C)(=O)=O)C[C@H]3F)ncc2C#N)s1] | LCMS calc. for C₁₈H₂₄FN₆O₃S₂ [M + H]⁺: m/z = 455.13; Found 455.3. |
| 154 | 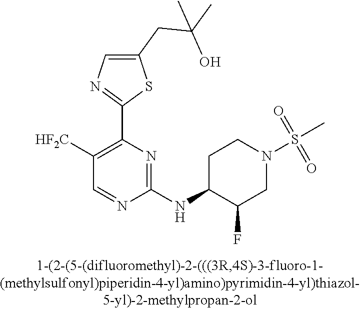 | LCMS calc. for C₁₈H₂₅F₃N₅O₃S₂ [M + H]⁺: m/z = 480.14; Found 480.4. |
| 155 | 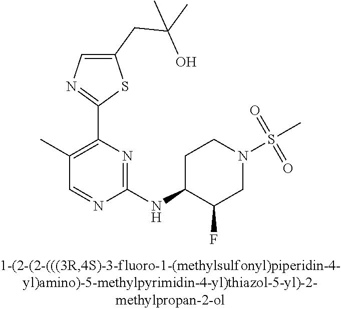 | LCMS calc. for C₁₈H₂₇FN₅O₃S₂ [M + H]⁺: m/z = 444.15; Found 444.4. |

### Example A—Enzymatic Activity and Cytotoxicity Studies

### CDK2/CyclinE2 Enzymatic Activity Assay

The inhibitory activity of compounds was evaluated in vitro using TR-FRET assay with white 384-well low volume microplate (Greiner Bio-One). CDK2/Cyclin E2 catalyzed phosphorylation of peptide in the presence and absence of compounds was measured and used in IC₅₀ determination. Recombinant protein complex CDK2/Cyclin E2, expressed from insect cell, was purchased from ProQinase. Testing compounds were dissolved in DMSO at 1 mM and tested in 9-dose IC₅₀ mode. The reaction mixture was prepared by mixing CDK2/CyclinE2 (1 nM final), ULight-4E-BP1 (50 nM final, Perkinelmer, TRF0128-D), and ATP (1 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by dispenser (TECAN D300E) to make a 9.9 μL of reaction mixture. After 20 minutes preincubation at room temperature, 0.1 μL MgCl₂ (10 mM final) was added to initiate the reaction. Following a 45 minute incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubated at room temperature for additional 60 minutes in the dark. The reaction signal was measured by Envision multimode plate reader (PerkinElmer, 2102-0010). IC₅₀ values were determined by fitting the data to the standard 4 parameters with Hill Slope using GraphPad Prism software.

### CDK4/CyclinD1 Enzymatic Activity Assay

The inhibitory activity of compounds was evaluated in vitro using TR-FRET assay with white 384-well low volume microplate (Greiner Bio-One). CDK4/Cyclin D1 catalyzed phosphorylation of peptide in the presence and absence of compounds was measured and used in IC₅₀ determination. Recombinant protein complex CDK4/Cyclin D1, expressed from insect cell, was purchased from ProQinase. Testing compounds were dissolved in DMSO at 1 mM and tested in 9-dose IC₅₀ mode. The reaction mixture was prepared by mixing CDK4/CyclinD1 (1 nM final), ULight-4E-BP1 (100 nM final, Perkinelmer, TRF0128-D), and ATP (2 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by the dispenser (TECAN D300E) to make a 9.9 μL of reaction mixture. After 20 minutes preincubation at room temperature, 0.1 μL MgCl₂ (10 mM final) was added to initiate the reaction. Following a 45-minute incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubated at room temperature for additional 60 minutes in dark. The reaction signal was measured by Envision multimode plate reader (PerkinElmer, 2102-0010). IC₅₀ values were determined by fitting the data to the standard 4 parameters with Hill Slope using GraphPad Prism software.

### CDK6/CyclinD1 Enzymatic Activity Assay

The inhibitory activity of compounds was evaluated in vitro using TR-FRET assay with white 384-well low volume microplate (Greiner Bio-One). CDK6/CyclinD1 catalyzed phosphorylation of peptide in the presence and absence of compounds was measured and used in IC₅₀ determination. Recombinant protein complex CDK6/CyclinD1 expressed from insect cell, was purchased from ProQinase. Testing compounds were dissolved in DMSO at 1 mM and tested in a 9-dose IC₅₀ mode. The reaction mixture was prepared by mixing CDK6/CyclinD1 (1 nM final), ULight-4E-BP1 (100 nM final, Perkinelmer, TRF0128-D), and ATP (1 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by dispenser (TECAN D300E) to make a 9.9 μL of reaction mixture. After 20 minutes preincubation at room temperature, 0.1 μL MgCl₂ (10 mM final) was added to initiate the reaction. Following a 40-minute incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubated at room temperature for additional 60 minutes in the dark. The reaction signal was measured by Envision multimode plate reader (PerkinElmer, 2102-0010). IC₅₀ values were determined by fitting the data to the standard 4 parameters with Hill Slope using GraphPad Prism software.

### Cell Proliferation Studies in OVCAR3 Cells

Cell proliferation studies were conducted in OVCAR3 adenocarcinoma cell line. Cells were maintained in RPMI (Corning, Catalog #: 10-040-CV) supplemented with 10% v/v FBS (Gibco, Catalog #: 26140-079), 1% v/v Penicillin Streptomycin (Gibco, Catalog #15140-122) Cells were seeded in 384-well plates at a density of 250 cells/well. Compounds dissolved in DMSO were plated in quadruplicate using a digital dispenser (D300E, Tecan) and tested with a 9-point 3-fold serial dilution. Cells were incubated for 10 days in a 37° C. active humidified incubator at 5% CO₂. A media exchange and second compound addition were performed on day 5. Cell viability was measured using the ATP-Lite 1-Step Luminescence reagent (Perkin Elmer, Catalog #: 6016731) as per manufacturer's instructions. Luminescence signal was measured with a multimode plate reader (Envision 2105, Perkin Elmer). Raw data files were imported to Dotmatics Screening Ultra for IC₅₀ analysis. Luminescence values were normalized to both background and DMSO controls to obtain a percentage of viable cells relative to DMSO vehicle control.

### pRB ICW Assay in OVCAR3 Cells

OVCAR3 cells were maintained in RPMI (Corning, Catalog #: 10-040-CV) supplemented with 10% v/v FBS (Gibco, Catalog #: 26140-079), 1% v/v Penicillin Streptomycin (Gibco, Catalog #15140-122.) OVCAR3 cells grown at log phase were trypsinized, counted, and resuspended in fresh medium to reach a final density of 6.7e4 cells/mL and 75 μL of culture were dispensed into a 384-well plate (Falcon, cat #353962) using a Multidrop Combi dispenser (Thermo Scientific). The next day, compounds were dispensed as a 9-point, ½ log serial dilution using a Tecan digital dispenser (D300e), and cells were incubated with compound for 2 hours in a humidified incubator at 37° C. A reference compound at a final concentration of 10 μM was used as a control for maximum inhibition. Each compound was tested in duplicate for each experiment. At the end of the incubation, L of 16% paraformaldehyde (Electron Microscopy, cat #15710) was slowly added to each well and the plate was incubated at room temperature (RT) for 30 minutes to fix cells. Cells were then permeabilized by incubating with 50 μL/well of wash buffer (1×PBS with 0.1% Triton X-100) 5×5 minutes, followed by 1 hour blocking with 30 μL/well of Odyssey blocking buffer (Li-COR, cat #927-40000), at RT. Anti-phosphor RB antibody (Cell signaling 8516S) was diluted 1:1000 in Odyssey blocking buffer and 20 μL was added to all wells and incubated overnight in 4° C. with gently rocking. The next day, cells were washed 5×5 min with 50 μL/well of wash buffer, followed with 1 hour incubation with secondary antibody and DRAQ5 diluted in Odyssey blocking buffer (1:500 dilution for secondary antibody and 1:2000 dilution for DRAQ5), 5×5 min washes, and one last wash with water. Plates were dried in 37° C. oven for 5 minutes and scanned using Li-COR Odyssey CLx imaging system to acquire intensities at 700 and 800 nm channels.

The IC₅₀ values are summarized in Table 8.

TABLE 8
IC₅₀ Values

| | CDK2_E1 | CDK4_D1 | OVCAR3 p-RB |
|---|---|---|---|
| Example | IC₅₀ (nM) | IC₅₀ (nM) | ICW IC₅₀ (nM) |
| 1 | +++ | +++ | +++ |
| 2 | +++ | +++ | +++ |
| 3 | +++ | +++ | +++ |
| 4 | +++ | +++ | ++ |
| 5 | +++ | +++ | ++ |
| 6 | +++ | +++ | +++ |
| 7 | +++ | +++ | +++ |
| 8 | +++ | +++ | +++ |
| 9 | +++ | +++ | +++ |
| 10 | +++ | ++ | ++ |
| 11 | +++ | +++ | +++ |
| 12 | +++ | +++ | +++ |
| 13 | +++ | +++ | +++ |
| 14 | +++ | +++ | ++ |
| 15 | +++ | +++ | +++ |
| 16 | +++ | +++ | +++ |
| 17 | +++ | +++ | +++ |
| 18 | +++ | +++ | +++ |
| 19 | +++ | +++ | +++ |
| 20 | +++ | +++ | ++ |
| 21 | +++ | ++ | ++ |
| 22 | +++ | ++ | +++ |
| 23 | +++ | +++ | +++ |
| 24 | +++ | +++ | +++ |
| 25 | +++ | +++ | ++ |
| 26 | +++ | +++ | +++ |
| 27 | +++ | +++ | ++ |
| 28 | +++ | +++ | +++ |
| 29 | +++ | +++ | ++ |
| 30 | +++ | +++ | +++ |
| 31 | +++ | +++ | +++ |
| 32 | +++ | +++ | +++ |
| 33 | +++ | +++ | +++ |
| 34 | +++ | +++ | +++ |
| 35 | +++ | +++ | +++ |
| 36 | +++ | +++ | +++ |
| 37 | +++ | +++ | +++ |
| 38 | +++ | ++ | ++ |
| 39 | +++ | +++ | +++ |
| 40 | +++ | ++ | ++ |
| 41 | +++ | +++ | +++ |
| 42 | +++ | +++ | +++ |
| 43 | +++ | ++ | + |
| 44 | +++ | +++ | +++ |
| 45 | +++ | +++ | +++ |
| 46 | +++ | +++ | +++ |
| 47 | +++ | +++ | ++ |
| 48 | +++ | +++ | +++ |
| 49 | +++ | +++ | ++ |
| 50 | +++ | +++ | +++ |
| 51 | +++ | +++ | +++ |
| 52 | +++ | ++ | ++ |
| 53 | +++ | ++ | + |
| 54 | +++ | + | + |
| 55 | +++ | +++ | ++ |
| 56 | +++ | +++ | +++ |
| 57 | +++ | +++ | +++ |
| 58 | +++ | +++ | ++ |
| 59 | +++ | +++ | +++ |
| 60 | +++ | +++ | +++ |
| 61 | +++ | +++ | +++ |
| 62 | +++ | +++ | +++ |
| 63 | +++ | +++ | +++ |
| 64 | +++ | ++ | + |
| 65 | +++ | +++ | ++ |
| 66 | +++ | +++ | +++ |
| 67 | +++ | +++ | +++ |
| 68 | +++ | ++ | ++ |
| 69 | +++ | ++ | +++ |
| 70 | +++ | +++ | +++ |
| 71 | +++ | +++ | +++ |
| 72 | +++ | +++ | +++ |
| 73 | +++ | +++ | +++ |
| 74 | +++ | +++ | +++ |
| 75 | +++ | + | + |
| 76 | ++ | ++ | + |
| 77 | +++ | + | + |
| 78 | ++ | + | + |
| 79 | +++ | +++ | ++ |
| 80 | +++ | +++ | ++ |
| 81 | +++ | +++ | +++ |
| 82 | +++ | ++ | + |
| 83 | +++ | +++ | ++ |
| 84 | +++ | +++ | +++ |
| 85 | +++ | ++ | ++ |
| 86 | +++ | + | + |
| 87 | +++ | +++ | +++ |
| 88 | +++ | +++ | +++ |
| 89 | +++ | +++ | +++ |
| 90 | +++ | +++ | +++ |
| 91 | +++ | ++ | ++ |
| 92 | + | + | + |
| 93 | +++ | ++ | ++ |
| 94 | +++ | + | ++ |
| 95 | +++ | ND | ++ |
| 96 | +++ | ND | +++ |
| 97 | +++ | ND | +++ |
| 98 | +++ | ND | +++ |
| 99 | +++ | ND | ++ |
| 100 | +++ | +++ | +++ |
| 101 | +++ | ND | +++ |
| 102 | +++ | ND | +++ |
| 103 | +++ | +++ | +++ |
| 104 | +++ | +++ | +++ |
| 105 | +++ | ND | +++ |
| 106 | +++ | ND | ++ |
| 107 | +++ | +++ | +++ |
| 108 | +++ | +++ | ++ |
| 109 | +++ | +++ | +++ |
| 110 | +++ | ND | ++ |
| 111 | +++ | ND | +++ |
| 112 | +++ | ND | +++ |
| 113 | +++ | ND | +++ |
| 114 | +++ | +++ | ++ |
| 115 | +++ | ND | +++ |
| 116 | +++ | ND | +++ |
| 117 | +++ | ND | +++ |
| 118 | +++ | ND | ++ |
| 119 | +++ | ND | +++ |
| 120 | +++ | ND | +++ |
| 121 | +++ | ND | ++ |
| 122 | +++ | ND | +++ |
| 123 | +++ | ND | +++ |
| 124 | +++ | ND | +++ |
| 125 | +++ | ND | ++ |
| 126 | +++ | ND | +++ |
| 127 | +++ | ND | +++ |
| 128 | +++ | ND | +++ |
| 129 | +++ | ND | +++ |
| 130 | +++ | ND | ++ |
| 131 | +++ | ++ | + |
| 132 | +++ | ++ | + |
| 133 | +++ | ND | +++ |
| 134 | +++ | ND | +++ |
| 135 | +++ | ND | ++ |
| 136 | +++ | ND | +++ |
| 137 | +++ | ND | +++ |
| 138 | +++ | ND | +++ |
| 139 | +++ | ND | +++ |
| 140 | +++ | ND | + |
| 141 | +++ | ND | ++ |
| 142 | +++ | ++ | ++ |
| 143 | +++ | ND | +++ |
| 144 | +++ | ND | ++ |
| 145 | +++ | ND | + |
| 146 | +++ | ND | ++ |
| 147 | +++ | ND | + |
| 148 | +++ | ND | + |
| 149 | +++ | ND | + |
| 150 | +++ | ND | ++ |
| 151 | +++ | ND | + |
| 152 | +++ | ND | ++ |
| 153 | +++ | ND | + |
| 154 | +++ | ND | + |
| 155 | ND | ND | +++ |

In Table 8, a “+” denotes an IC₅₀ value of >500 nM; a “++” denotes an IC₅₀ value of 100 nM < IC₅₀ ≤ 500 nM; a “+++” denotes an IC₅₀ value of IC₅₀ ≤ 100 nM; ND = not determined.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula I: 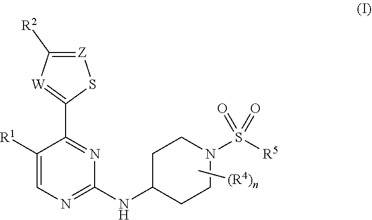
or a pharmaceutically acceptable salt or solvate or N-oxide thereof, wherein
Z is N or CR⁶;
W is N or CR⁶;
n is 0, 1, 2, 3, 4, 5, 6, 7, 8, or 9;
R¹ is selected from H, D, halogen, C₁-C₆ alkyl, C₁-C₆ alkoxide, C₁-C₆ haloalkyl, SF₅, or CN; wherein said C₁-C₆ alkyl is optionally substituted by 1-6 R groups selected from H, D, halogen, —OH, —CN, —OR^{a}, —SR^{a}, —NR^{c}R^{d};
R² is selected from H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R, —P(O)(OR^{b})(OR^{b}), —B(OR^{c})(OR^{d}) or —S(O)₂R^{b};
each R⁴ is independently H, D, halogen, —OH, —CN, —NO₂, —C₁-C₆alkyl, —C₁-C₆ haloalkyl, —C₁₋₆alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R, —P(O)(OR^{b})(OR^{b}), —B(OR^{c})(OR^{d}) or —S(O)₂R^{b};
or two R⁴ together with the same atom to which they are both attached is —C(O)— or —C(S)—;
or two R⁴ together with the carbon atom(s) to which they are both attached at same carbon or different carbons, form a carbocyclic or heterocyclic group which is optionally substituted by 1-6 R groups selected from H, D, halogen, —OH, —CN, —OR^{a}, —SR^{a}, —R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
each R^{a} is independently H, D, —C(O)R^{b}, —C(O)OR^{c}, —C(O)NR^{c}R^{d}, —C(═NR)NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl; wherein said —C₁-C₁₀alkyl, —C₂-C₁₀ alkenyl, —C₂-C₁₀ alkynyl, C₀-C₁alk-aryl, cycloalkyl, cycloalkenyl, C₀-C₁alk-heteroaryl, heterocycloalkyl, or heterocycloalkenyl is optionally substituted;
each R^{b}, is independently H, D, —C₁-C₆ alkyl, —C₁-C₆ haloalkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, C₁alk-aryl, heteroaryl, cycloalkyl, cycloalkenyl, C₁alk-heteroaryl, heterocycloalkyl, or heterocycloalkenyl;
or R^{c} and R^{d}, together with the atom to which they are both attached, form an optionally substituted monocyclic or multicyclic heterocycloalkyl, or an optionally substituted monocyclic or multicyclic heterocyclo-alkenyl group;
R⁵ is —NR^{c}R^{d}, —NR^{a}R^{c}, C₁₋₆alkyl, C₃₋₇cycloalkyl, C₄₋₇heterocycloalkyl, C₃-7cycloalkylalkyl, C₄₋₇heterocycloalkylalkyl, aryl, heteroaryl, arylalkyl, heteroarylalkyl, or haloalkyl; wherein said C₁₋₆alkyl, C₃₋₇cycloalkyl, C₄₋₇heterocycloalkyl, C₃₋₇cycloalkylalkyl, C₄-7heterocycloalkylalkyl, aryl, heteroaryl, arylalkyl and heteroarylalkyl is optionally substituted by 1-6 R groups selected from H, D, halogen, —OH, —CN, —OR^{a}, —SR^{a}, —R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or R⁴ and R⁵ together with the atoms to which they are both attached form a heterocyclic group which are optionally substituted with halogen, —OH, —CN, —OR^{a}, —SR^{a}, —R^{b}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; and
each R⁶ is independently H, D, halogen, —OH, CN, NO₂, C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, heteroarylalkyl, OR^{a}, SR^{a}, NR^{c}R^{d}, NR^{a}R^{c}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R, —P(O)(OR^{b})(OR^{b}), —B(OR^{c})(OR^{d}) or —S(O)₂R^{b}; wherein said C₁-C₆alkyl, C₂-C₆alkenyl, C₂-C₆alkynyl, aryl, heteroaryl, cycloalkyl, cycloalkenyl, heterocycloalkyl, heterocycloalkenyl, cycloalkylalkyl, heterocycloalkylalkyl, arylalkyl, or heteroarylalkyl is optionally substituted by 1-6 R groups selected from H, D, Oxo, halogen, —OH, —CN, —OR^{a}, —SR^{a}, —NR^{c}R^{d}, —NR^{a}R^{c}, —R^{b}, —C(O)R^{b}, —OC(O)R^{b}, —C(O)OR^{b}, —C(O)NR^{c}R^{d}, —C(═NR^{b})NR^{c}R^{d}, —C(═NR^{b})R^{b}, —S(O)R^{b}, —S(O)₂NR^{c}R^{d}, —S(O)(═NR^{b})R^{b}, —SF₅, —P(O)R^{b}R, —P(O)(OR^{b})(OR^{b}), —B(OR^{c})(OR^{d}) or —S(O)₂R^{b}.

2-15. (canceled)

16. The compound of claim 1, wherein both W and Z are CR⁶: or wherein W is CR⁶ and Z is N: or wherein W is N and Z are CR⁶.

17-23. (canceled)

24. The compound of claim 1, wherein R₅ is C₁₋₆alkyl, aryl or heteroaryl.

25-28. (canceled)

29. The compound of claim 1, wherein R₂ is H, —CN or C₁-C₆ alkyl optionally substituted by 1-6 R groups selected from H, D, halogen, —OH, —CN, —OR^{a}, —SR^{a}, —NR^{c}R^{d}, cycloalkyl, heterocycloalkyl, —C(O)NR^{c}R^{d}, —C(═NR)NR^{b}R^{c}, —C(O)OR^{b}, —C(O)R^{b}, —C(═NR^{b})R^{b}, S(O)₂R^{b}, and S(O)R^{b}.

30. (canceled)

31. The compound of claim 1, wherein R₁ is F, CH₂F or CF₃.

32. (canceled)

33. The compound of claim 1, that is a compound of formula II:

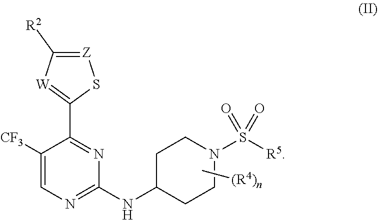

34. The compound of claim 1, that is a compound of formula III:

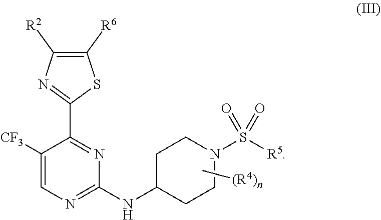

35. The compound of claim 1, that is a compound of formula IV:

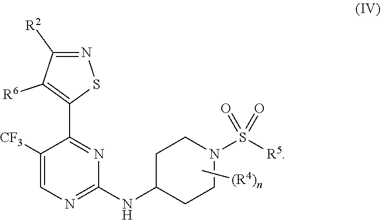

36. The compound of claim 1, that is a compound of formula V:

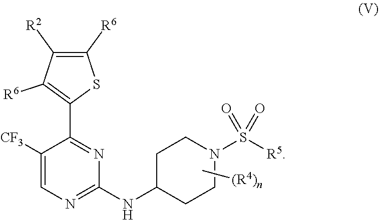

37. The compound of claim 1, that is a compound of formula VI:

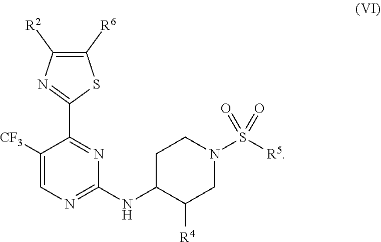

38. The compound of claim 1, that is a compound of formula VII:

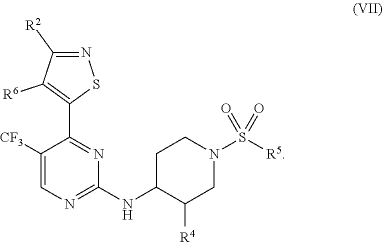

39. The compound of claim 1, that is a compound of formula VIII:

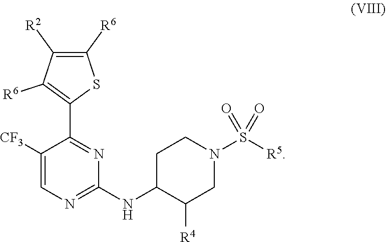

40. The compound of claim 1, that is:
2-Methyl-5-[2-[[1-(1-methylimidazol-4-yl)sulfonylpiperidin-4-yl]amino]-5-(trifluoro-methyl)pyrimidin-4-yl]thiophene-3-carbonitrile;
N-(1-((1-methyl-1H-pyrazol-4-yl)sulfonyl)piperidin-4-yl)-4-(5-methylthiazol-2-yl)-5-(trifluoromethyl)pyrimidin-2-amine;
2-Methyl-1-(2-(2-((1-((1-methyl-1H-imidazol-4-yl)sulfonyl)piperidin-4-yl)amino)-5-(trifluoromethyl)pyrimidin-4-yl)thiazol-5-yl)propan-2-ol;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carbonitrile;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-methylthiophene-3-carbonitrile;
5-(2-(((3R,4R)-3-fluoro-1-(methylsulfonyl)piperidin-4-yl)amino)-5-(trifluoromethyl)-pyrimidin-4-yl)thiophene-3-carboxamide;
5-(2-(((3R,4S)-3-fluoro-1-(methylsulfonyl)piperidin-4-yl)amino)-5-(trifluoromethyl)-pyrimidin-4-yl)thiophene-3-carboxamide;
5-(2-(((3R,4R)-3-fluoro-1-(methylsulfonyl)piperidin-4-yl)amino)-5-(trifluoromethyl)-pyrimidin-4-yl)-2-methylthiophene-3-carboxamide;
5-(2-(((3R,4S)-3-fluoro-1-(methylsulfonyl)piperidin-4-yl)amino)-5-(trifluoromethyl)-pyrimidin-4-yl)-2-methylthiophene-3-carboxamide;
5-(2-(((3R,4S)-3-methyl-1-(methylsulfonyl)piperidin-4-yl)amino)-5-(trifluoromethyl)-pyrimidin-4-yl)thiophene-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiophene-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiophene-3-carboxamide;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-methylthiophene-3-carboxamide;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carboxamide;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-methylthiophene-3-carboxamide;
5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carboxamide;
5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-1-((1H-imidazol-4-yl)sulfonyl)-3-methylpiperidin-4-yl)amino)-5-(trifluoromethyl)pyrimidin-4-yl)thiophene-3-carboxamide;
5-(2-(((3R,4S)-1-(imidazo[1,2-b]pyridazin-3-ylsulfonyl)-3-methylpiperidin-4-yl)amino)-5-(trifluoromethyl)pyrimidin-4-yl)thiophene-3-carboxamide;
2-methyl-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carboxamide;
2-methyl-5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-1-((1H-imidazol-4-yl)sulfonyl)-3-methylpiperidin-4-yl)amino)-5-(trifluoro-methyl)pyrimidin-4-yl)-2-methylthiophene-3-carboxamide;
N-((3R,4S)-3-fluoro-1-((1-methyl-1H-pyrazol-4-yl)sulfonyl)piperidin-4-yl)-4-(5-methylthiazol-2-yl)-5-(trifluoromethyl)pyrimidin-2-amine;
N-((3R,4R)-3-fluoro-1-((1-methyl-1H-pyrazol-4-yl)sulfonyl)piperidin-4-yl)-4-(5-methylthiazol-2-yl)-5-(trifluoromethyl)pyrimidin-2-amine;
N-((3R,4S)-3-fluoro-1-((1-methyl-1H-imidazol-4-yl)sulfonyl)piperidin-4-yl)-4-(5-methylthiazol-2-yl)-5-(trifluoromethyl)pyrimidin-2-amine;
N-((3R,4R)-3-fluoro-1-((1-methyl-1H-imidazol-4-yl)sulfonyl)piperidin-4-yl)-4-(5-methylthiazol-2-yl)-5-(trifluoromethyl)pyrimidin-2-amine;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-methylthiophene-3-carbonitrile;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-methylthiophene-3-carbonitrile;
N-(1-((1-methyl-1H-imidazol-4-yl)sulfonyl)piperidin-4-yl)-4-(5-methylthiazol-2-yl)-5-(trifluoromethyl)pyrimidin-2-amine;
2-(1-hydroxyethyl)-5-(2-((1-((1-methyl-1H-imidazol-4-yl)sulfonyl)piperidin-4-yl)amino)-5-(trifluoromethyl)pyrimidin-4-yl)thiophene-3-carbonitrile;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-(1-hydroxyethyl)thiophene-3-carbonitrile;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-(1-hydroxyethyl)thiophene-3-carbonitrile;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-(1-hydroxyethyl)thiophene-3-carbonitrile;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-(1-hydroxyethyl)thiophene-3-carbonitrile;
2-(2-hydroxy-2-methylpropyl)-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carbonitrile;
2-(2-hydroxy-2-methylpropyl)-5-(2-((1-((1-methyl-1H-imidazol-4-yl)sulfonyl)piperidin-4-yl)amino)-5-(trifluoromethyl)pyrimidin-4-yl)thiophene-3-carbonitrile;
2-(2-hydroxy-2-methylpropyl)-5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carbonitrile;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carbonitrile;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carbonitrile;
2-(2-hydroxy-2-methylpropyl)-5-(2-(((3R,4S)-3-methyl-1-(methylsulfonyl)piperidin-4-yl)amino)-5-(trifluoromethyl)pyrimidin-4-yl)thiophene-3-carboxamide;
2-(2-hydroxy-2-methylpropyl)-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carboxamide;
2-(2-hydroxy-2-methylpropyl)-5-(2-((1-((1-methyl-1H-imidazol-4-yl)sulfonyl)piperidin-4-yl)amino)-5-(trifluoromethyl)pyrimidin-4-yl)thiophene-3-carboxamide;
5-(2-(((3R,4S)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-(((3R,4R)-3-fluoro-1-((1-methyl-1H-pyrazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carboxamide;
5-(2-((1-((1-methyl-1H-imidazol-4-yl)sulfonyl)piperidin-4-yl)amino)-5-(trifluoro-methyl)pyrimidin-4-yl)isothiazole-3-carboxamide;
2-(2-hydroxy-2-methylpropyl)-5-(2-((1-((1-methyl-1H-pyrazol-4-yl)sulfonyl)piperidin-4-yl)amino)-5-(trifluoromethyl)pyrimidin-4-yl)thiophene-3-carboxamide;
2-methyl-5-(2-(((3R,4S)-3-methyl-1-(methylsulfonyl)piperidin-4-yl)amino)-5-(trifluoro-methyl)pyrimidin-4-yl)thiophene-3-carboxamide;
2-methyl-5-(2-(((3R,4S)-3-methyl-1-((1-methyl-1H-imidazol-4-yl)sulfonyl)piperidin-4-yl)amino)-5-(trifluoromethyl)pyrimidin-4-yl)thiophene-3-carbonitrile;
2-methyl-5-(2-(((3R,4S)-3-methyl-1-(methylsulfonyl)piperidin-4-yl)amino)-5-(trifluoro-methyl)pyrimidin-4-yl)thiophene-3-carbonitrile;
5-(2-((1-((1H-imidazol-4-yl)sulfonyl)piperidin-4-yl)amino)-5-(trifluoromethyl)pyrimidin-4-yl)-2-methylthiophene-3-carbonitrile;
1-(2-(2-((1-((1H-pyrazol-4-yl)sulfonyl)piperidin-4-yl)amino)-5-(trifluoromethyl)pyrimidin-4-yl)thiazol-5-yl)ethan-1-ol;
1-(2-(2-((1-((1H-imidazol-4-yl)sulfonyl)piperidin-4-yl)amino)-5-(trifluoromethyl)pyrimidin-4-yl)thiazol-5-yl)ethan-1-ol;
N-(1-((1-methyl-1H-pyrazol-4-yl)sulfonyl)piperidin-4-yl)-4-(thiazol-2-yl)-5-(trifluoro-methyl)pyrimidin-2-amine;
N-(1-((1-methyl-1H-imidazol-4-yl)sulfonyl)piperidin-4-yl)-4-(thiazol-2-yl)-5-(trifluoro-methyl)pyrimidin-2-amine;
2,2,2-trifluoro-1-(2-(2-((1-((1-methyl-1H-imidazol-4-yl)sulfonyl)piperidin-4-yl)amino)-5-(trifluoromethyl)pyrimidin-4-yl)thiazol-5-yl)ethan-1-ol;
2,2,2-trifluoro-1-(2-(2-((1-((1-methyl-1H-pyrazol-4-yl)sulfonyl)piperidin-4-yl)amino)-5-(trifluoromethyl)pyrimidin-4-yl)thiazol-5-yl)ethan-1-ol;
2-methyl-1-(2-(2-((1-((1-methyl-1H-pyrazol-4-yl)sulfonyl)piperidin-4-yl)amino)-5-(trifluoromethyl)pyrimidin-4-yl)thiazol-5-yl)propan-2-ol;
1-(2-(2-((1-((1-methyl-1H-imidazol-4-yl)sulfonyl)piperidin-4-yl)amino)-5-(trifluoromethyl)-pyrimidin-4-yl)thiazol-5-yl)ethan-1-ol;
N-((3R,4S)-3-methyl-1-((1-methyl-1H-imidazol-4-yl)sulfonyl)piperidin-4-yl)-4-(5-methylthiazol-2-yl)-5-(trifluoromethyl)pyrimidin-2-amine;
N-(1-(methylsulfonyl)piperidin-4-yl)-4-(5-methylthiazol-2-yl)-5-(trifluoromethyl)pyrimidin-2-amine;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)isothiazole-3-carboxamide;
5-(2-(((3R,4S)-3-fluoro-1-((1-methyl-1H-imidazol-4-yl)sulfonyl)piperidin-4-yl)amino)-5-(trifluoromethyl)pyrimidin-4-yl)isothiazole-3-carboxamide;
5-(2-(((3R,4S)-1-((1H-imidazol-4-yl)sulfonyl)-3-methylpiperidin-4-yl)amino)-5-(trifluoro-methyl)pyrimidin-4-yl)-2-(1-hydroxyethyl)thiophene-3-carbonitrile;
2-methyl-5-(2-(((3R,4S)-3-methyl-1-((1-methyl-1H-pyrazol-4-yl)sulfonyl)piperidin-4-yl)amino)-5-(trifluoromethyl)pyrimidin-4-yl)thiophene-3-carbonitrile;
5-(2-((1-((1-Methyl-1H-pyrazol-4-yl)sulfonyl)piperidin-4-yl)amino)-5-(trifluoro-methyl)pyrimidin-4-yl)thiophene-3-carbonitrile;
2-methyl-5-(2-((1-(methylsulfonyl)piperidin-4-yl)amino)-5-(trifluoromethyl)pyrimidin-4-yl)thiophene-3-carboxamide;
2-methyl-5-(2-((1-((1-methyl-1H-pyrazol-4-yl)sulfonyl)piperidin-4-yl)amino)-5-(trifluoro-methyl)pyrimidin-4-yl)thiophene-3-carboxamide;
2-methyl-5-(2-((1-((1-methyl-1H-pyrazol-4-yl)sulfonyl)piperidin-4-yl)amino)-5-(trifluoro-methyl)pyrimidin-4-yl)thiophene-3-carbonitrile;
5-(2-((1-(methylsulfonyl)piperidin-4-yl)amino)-5-(trifluoromethyl)pyrimidin-4-yl)thiophene-3-carboxamide;
5-(2-((1-((1-methyl-1H-pyrazol-4-yl)sulfonyl)piperidin-4-yl)amino)-5-(trifluoromethyl)-pyrimidin-4-yl)thiophene-3-carboxamide;
4-(4-methoxy-5-methylthiophen-2-yl)-N-(1-((1-methyl-1H-imidazol-4-yl)sulfonyl)piperidin-4-yl)-5-(trifluoromethyl)pyrimidin-2-amine;
4-(4-methoxy-5-methylthiophen-2-yl)-N-(1-(methylsulfonyl)piperidin-4-yl)-5-(trifluoro-methyl)pyrimidin-2-amine;
N-(1-((1-methyl-1H-pyrazol-4-yl)sulfonyl)piperidin-4-yl)-4-(3-methylisothiazol-5-yl)-5-(trifluoromethyl)pyrimidin-2-amine;
4-(3-methylisothiazol-5-yl)-N-(1-(methylsulfonyl)piperidin-4-yl)-5-(trifluoromethyl)-pyrimidin-2-amine;
4-(4-(difluoromethyl)-5-methylthiophen-2-yl)-N-(1-((1-methyl-1H-pyrazol-4-yl)sulfonyl)-piperidin-4-yl)-5-(trifluoromethyl)pyrimidin-2-amine;
4-(4-(difluoromethyl)-5-methylthiophen-2-yl)-N-(1-(methylsulfonyl)piperidin-4-yl)-5-(trifluoromethyl)pyrimidin-2-amine;
1-(2-methyl-5-(2-((1-((1-methyl-1H-pyrazol-4-yl)sulfonyl)piperidin-4-yl)amino)-5-(trifluoromethyl)pyrimidin-4-yl)thiophen-3-yl)ethan-1-one;
4-(4-(methoxymethyl)-5-methylthiophen-2-yl)-N-(1-((1-methyl-1H-pyrazol-4-yl)sulfonyl)piperidin-4-yl)-5-(trifluoromethyl)pyrimidin-2-amine;
2-methyl-5-(2-((1-(methylsulfonyl)piperidin-4-yl)amino)-5-(trifluoromethyl)pyrimidin-4-yl)thiophene-3-carbonitrile;
5-(2-((1-(methylsulfonyl)piperidin-4-yl)amino)-5-(trifluoromethyl)pyrimidin-4-yl)isothiazole-3-carboxamide;
5-(2-(((3R,4S)-1-((1H-imidazol-4-yl)sulfonyl)-3-methylpiperidin-4-yl)amino)-5-(trifluoro-methyl)pyrimidin-4-yl)-2-methylthiophene-3-carbonitrile;
5-(2-(((3R,4S)-1-((1-(Cyanomethyl)-1H-pyrazol-4-yl)sulfonyl)-3-methylpiperidin-4-yl)amino)-5-(trifluoromethyl)pyrimidin-4-yl)thiophene-3-carboxamide;
5-(2-((1-((1-(cyanomethyl)-1H-pyrazol-4-yl)sulfonyl)piperidin-4-yl)amino)-5-(trifluoro-methyl)pyrimidin-4-yl)-2-methylthiophene-3-carbonitrile;
1-(2-(2-((1-((1-Methyl-1H-pyrazol-4-yl)sulfonyl)piperidin-4-yl)amino)-5-(trifluoromethyl)-pyrimidin-4-yl)thiazol-5-yl)ethan-1-ol;
2-(1-Hydroxyethyl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile;
5-(2-((1-((1-methyl-1H-pyrazol-4-yl)sulfonyl)piperidin-4-yl)amino)-5-(trifluoromethyl)-pyrimidin-4-yl)isothiazole-3-carbonitrile;
2-(2-((1-((1-Methyl-1H-imidazol-4-yl)sulfonyl)piperidin-4-yl)amino)-5-(trifluoromethyl)-pyrimidin-4-yl)thiazole-4-carboxamide;
5-(2-((1-((2-Aminothiazol-5-yl)sulfonyl)piperidin-4-yl)amino)-5-(trifluoromethyl)pyrimidin-4-yl)-2-methylthiophene-3-carbonitrile;
5-(2-(((3R,4S)-1-((2-aminothiazol-5-yl)sulfonyl)-3-methylpiperidin-4-yl)amino)-5-(trifluoro-methyl)pyrimidin-4-yl)-2-methylthiophene-3-carbonitrile;
or a pharmaceutically acceptable salt thereof.

41. The compound of claim 1, that is:
2-(2-(2-((1-(methylsulfonyl)piperidin-4-yl)amino)-5-(trifluoromethyl)pyrimidin-4-yl)thiazol-5-yl)propan-2-ol;
2-(2-(2-(((3R,4S)-3-fluoro-1-(methylsulfonyl)piperidin-4-yl)amino)-5-(trifluoromethyl) pyrimidin-4-yl)thiazol-5-yl)propan-2-ol;
2-(2-(2-(((3R,4R)-3-fluoro-1-(methylsulfonyl)piperidin-4-yl)amino)-5-98(trifluoromethyl) pyrimidin-4-yl)thiazol-5-yl)propan-2-ol;
2-(2-(2-((1-((1-methyl-1H-imidazol-4-yl)sulfonyl)piperidin-4-yl)amino)-5-(trifluoromethyl) pyrimidin-4-yl)thiazol-5-yl)propan-2-ol;
2-(2-(2-((1-((1-methyl-1H-pyrazol-4-yl)sulfonyl)piperidin-4-yl)amino)-5-(trifluoromethyl) pyrimidin-4-yl)thiazol-5-yl)propan-2-ol;
2-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
2-(2-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiazol-5-yl)propan-2-ol;
1,1-difluoro-2-(2-(2-((1-(methylsulfonyl)piperidin-4-yl)amino)-5-(trifluoromethyl) pyrimidin-4-yl)thiazol-5-yl)propan-2-ol;
1,1,1-trifluoro-2-(2-(2-((1-(methylsulfonyl)piperidin-4-yl)amino)-5-(trifluoromethyl) pyrimidin-4-yl)thiazol-5-yl)propan-2-ol;
1,1-difluoro-2-(2-(2-(((3R,4S)-3-methyl-1-((1-methyl-1H-imidazol-4-yl)sulfonyl)piperidin-4-yl)amino)-5-(trifluoromethyl)pyrimidin-4-yl)thiazol-5-yl)propan-2-ol;
1,1,1-trifluoro-2-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
1-(2-(2-((1-(methylsulfonyl)piperidin-4-yl)amino)-5-(trifluoromethyl)pyrimidin-4-yl)thiazol-5-yl)cyclobutan-1-ol;
2-methyl-1-(2-(2-((1-(methylsulfonyl)piperidin-4-yl)amino)-5-(trifluoromethyl)pyrimidin-4-yl)thiazol-5-yl)propan-2-ol;
1-(2-(2-(((3R,4S)-3-fluoro-1-(methylsulfonyl)piperidin-4-yl)amino)-5-(trifluoromethyl) pyrimidin-4-yl)thiazol-5-yl)-2-methylpropan-2-ol;
1-(2-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiazol-5-yl)-2-methylpropan-2-ol;
1-(2-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiazol-5-yl)-2-methylpropan-2-ol;
2-methyl-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
1-(2-(2-((1-(methylsulfonyl)piperidin-4-yl)amino)-5-(trifluoromethyl)pyrimidin-4-yl)thiazol-5-yl)ethan-1-ol;
2,2,2-trifluoro-1-(2-(2-((1-(methylsulfonyl)piperidin-4-yl)amino)-5-(trifluoromethyl) pyrimidin-4-yl)thiazol-5-yl)ethan-1-ol;
1-(2-(2-(((3R,4S)-3-fluoro-1-((1-methyl-1H-pyrazol-4-yl)sulfonyl)piperidin-4-yl)amino)-5-(trifluoromethyl)pyrimidin-4-yl)thiazol-5-yl)ethan-1-ol;
1-(2-(2-(((3R,4R)-3-fluoro-1-((1-methyl-1H-pyrazol-4-yl)sulfonyl)piperidin-4-yl)amino)-5-(trifluoromethyl)pyrimidin-4-yl)thiazol-5-yl)ethan-1-ol;
5-(2-(((3R,4R)-3-fluoro-1-(methylsulfonyl)piperidin-4-yl)amino)-5-(trifluoromethyl) pyrimidin-4-yl)-2-(2-hydroxypropan-2-yl)thiophene-3-carbonitrile;
2-(2-hydroxypropan-2-yl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile;
2-(2-hydroxypropan-2-yl)-5-(2-(((3R,4S)-3-methyl-1-((1-methyl-1H-pyrazol-4-yl)sulfonyl) piperidin-4-yl)amino)-5-(trifluoromethyl)pyrimidin-4-yl)thiophene-3-carbonitrile;
5-(2-(((3R,4R)-3-fluoro-1-((1-methyl-H-pyrazol-4-yl)sulfonyl)piperidin-4-yl)amino)-5-(trifluoromethyl)pyrimidin-4-yl)-2-(2-hydroxypropan-2-yl)thiophene-3-carbonitrile;
5-(2-(((3R,4S)-1-((1H-imidazol-4-yl)sulfonyl)-3-methylpiperidin-4-yl)amino)-5-(trifluoromethyl)pyrimidin-4-yl)-2-(2-hydroxypropan-2-yl)thiophene-3-carbonitrile;
2-(2-hydroxypropan-2-yl)-5-(2-(((3R,4S)-3-methyl-1-((1-methyl-1H-imidazol-4-yl)sulfonyl) piperidin-4-yl)amino)-5-(trifluoromethyl)pyrimidin-4-yl)thiophene-3-carbonitrile;
5-(2-((1-(methylsulfonyl)piperidin-4-yl)amino)-5-(trifluoromethyl)pyrimidin-4-yl)-2-(2,2,2-trifluoro-1-hydroxyethyl)thiophene-3-carbonitrile;
5-(2-(((3R,4S)-3-methyl-1-((1-methyl-H-pyrazol-4-yl)sulfonyl)piperidin-4-yl)amino)-5-(trifluoromethyl)pyrimidin-4-yl)-2-(2,2,2-trifluoro-1-hydroxyethyl)thiophene-3-carbonitrile;
2-(2-hydroxy-2-methylpropyl)-5-(2-((1-(methylsulfonyl)piperidin-4-yl)amino)-5-(trifluoromethyl)pyrimidin-4-yl)thiophene-3-carbonitrile;
2-(2-hydroxy-2-methylpropyl)-5-(2-((1-((1-methyl-1H-pyrazol-4-yl)sulfonyl)piperidin-4-yl)amino)-5-(trifluoromethyl)pyrimidin-4-yl)thiophene-3-carbonitrile;
5-(2-(((3R,4R)-3-fluoro-1-((1-methyl-H-pyrazol-4-yl)sulfonyl)piperidin-4-yl)amino)-5-(trifluoromethyl)pyrimidin-4-yl)-2-(2-hydroxy-2-methylpropyl)thiophene-3-carbonitrile;
5-(2-(((3R,4R)-3-fluoro-1-((1-methyl-1H-imidazol-4-yl)sulfonyl)piperidin-4-yl)amino)-5-(trifluoromethyl)pyrimidin-4-yl)-2-(2-hydroxy-2-methylpropyl)thiophene-3-carbonitrile;
5-(2-(((3R,4R)-3-fluoro-1-((1-methyl-H-pyrazol-4-yl)sulfonyl)piperidin-4-yl)amino)-5-(trifluoromethyl)pyrimidin-4-yl)isothiazole-3-carboxamide;
5-(2-(((3R,4S)-3-fluoro-1-((1-methyl-H-pyrazol-4-yl)sulfonyl)piperidin-4-yl)amino)-5-(trifluoromethyl)pyrimidin-4-yl)isothiazole-3-carboxamide;
2-(2-hydroxy-2-methylpropyl)-5-(2-((1-(methylsulfonyl)piperidin-4-yl)amino)-5-(trifluoromethyl)pyrimidin-4-yl)thiophene-3-carboxamide;
2-methyl-1-(2-(2-(((3R,4S)-3-methyl-1-(methylsulfonyl)piperidin-4-yl)amino)-5-(trifluoromethyl)pyrimidin-4-yl)thiazol-5-yl)propan-2-ol;
2-(2-(2-(((3R,4S)-3-methyl-1-(methylsulfonyl)piperidin-4-yl)amino)-5-(trifluoromethyl) pyrimidin-4-yl)thiazol-5-yl)propan-2-ol;
2-(2-(2-(((3R,4S)-3-methyl-1-((1-methyl-1H-imidazol-4-yl)sulfonyl)piperidin-4-yl)amino)-5-(trifluoromethyl)pyrimidin-4-yl)thiazol-5-yl)propan-2-ol;
2,2-difluoro-1-(2-(2-((1-(methylsulfonyl)piperidin-4-yl)amino)-5-(trifluoromethyl) pyrimidin-4-yl)thiazol-5-yl)ethan-1-ol;
1-(2-(2-(((3R,4R)-3-fluoro-1-(methylsulfonyl)piperidin-4-yl)amino)-5-(trifluoromethyl) pyrimidin-4-yl)thiazol-5-yl)-2-methylpropan-2-ol;
1-(2-(2-(((3R,4S)-3-fluoro-1-((1-methyl-1H-imidazol-4-yl)sulfonyl)piperidin-4-yl)amino)-5-(trifluoromethyl)pyrimidin-4-yl)thiazol-5-yl)-2-methylpropan-2-ol;
1-(2-(2-(((3R,4S)-1-((1H-pyrazol-4-yl)sulfonyl)-3-fluoropiperidin-4-yl)amino)-5-(trifluoromethyl)pyrimidin-4-yl)thiazol-5-yl)-2-methylpropan-2-ol;
2,2-difluoro-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)ethan-1-ol;
2,2-difluoro-1-(2-(2-(((3R,4S)-3-methyl-1-((1-methyl-1H-imidazol-4-yl)sulfonyl)piperidin-4-yl)amino)-5-(trifluoromethyl)pyrimidin-4-yl)thiazol-5-yl)ethan-1-ol;
2,2,2-trifluoro-1-(2-(2-(((3R,4S)-3-methyl-1-(methylsulfonyl)piperidin-4-yl)amino)-5-(trifluoromethyl)pyrimidin-4-yl)thiazol-5-yl)ethan-1-ol;
2,2,2-trifluoro-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)ethan-1-ol;
1,1-difluoro-2-(2-(2-(((3R,4S)-3-methyl-1-(methylsulfonyl)piperidin-4-yl)amino)-5-(trifluoromethyl)pyrimidin-4-yl)thiazol-5-yl)propan-2-ol;
1,1-difluoro-2-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)propan-2-ol;
2-fluoro-1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)ethan-1-ol;
2-fluoro-1-(2-(2-(((3R,4S)-3-methyl-1-(methylsulfonyl)piperidin-4-yl)amino)-5-(trifluoromethyl)pyrimidin-4-yl)thiazol-5-yl)ethan-1-ol;
2,2-difluoro-1-(2-(2-(((3R,4S)-3-methyl-1-(methylsulfonyl)piperidin-4-yl)amino)-5-(trifluoromethyl)pyrimidin-4-yl)thiazol-5-yl)ethan-1-ol;
2-methyl-1-(2-(2-(((3R,4S)-3-methyl-1-(morpholinosulfonyl)piperidin-4-yl)amino)-5-(trifluoromethyl)pyrimidin-4-yl)thiazol-5-yl)propan-2-ol;
2-methyl-1-(2-(2-(((3R,4S)-3-methyl-1-((4-methylpiperazin-1-yl)sulfonyl)piperidin-4-yl)amino)-5-(trifluoromethyl)pyrimidin-4-yl)thiazol-5-yl)propan-2-ol;
(3R,4S)-4-((4-(5-(2-hydroxy-2-methylpropyl)thiazol-2-yl)-5-(trifluoromethyl)pyrimidin-2-yl)amino)-N,N,3-trimethylpiperidine-1-sulfonamide;
1-(2-(2-(((3R,4S)-3-methyl-1-((1-methyl-1H-pyrazol-4-yl)sulfonyl)piperidin-4-yl)amino)-5-(trifluoromethyl)pyrimidin-4-yl)thiazol-5-yl)cyclopropan-1-ol;
1-(2-(2-(((3R,4S)-3-methyl-1-(methylsulfonyl)piperidin-4-yl)amino)-5-(trifluoromethyl)pyrimidin-4-yl)thiazol-5-yl)cyclopropan-1-ol;
1-(2-(5-chloro-2-(((3R,4S)-3-fluoro-1-(methylsulfonyl)piperidin-4-yl)amino)pyrimidin-4-yl)thiazol-5-yl)-2-methylpropan-2-ol;
2-(((3R,4S)-3-fluoro-1-(methylsulfonyl)piperidin-4-yl)amino)-4-(5-(2-hydroxy-2-methylpropyl)thiazol-2-yl)pyrimidine-5-carbonitrile;
1-(2-(5-(difluoromethyl)-2-(((3R,4S)-3-fluoro-1-(methylsulfonyl)piperidin-4-yl)amino)pyrimidin-4-yl)thiazol-5-yl)-2-methylpropan-2-ol;
1-(2-(2-(((3R,4S)-3-fluoro-1-(methylsulfonyl)piperidin-4-yl)amino)-5-methylpyrimidin-4-yl)thiazol-5-yl)-2-methylpropan-2-ol;
or a pharmaceutically acceptable salt thereof.

42. A pharmaceutical composition comprising a compound according to any one of the preceding claims, or a pharmaceutically acceptable salt thereof, and a pharmaceutically acceptable excipient.

43. (canceled)

44. A method for treating a disorder mediated by CDK2 or CDK4 in a patient in need thereof, comprising administering to said patient a compound according to claim 1, or a pharmaceutically acceptable salt thereof, or a pharmaceutical composition comprising the compound;
optionally, wherein the disorder is a cancer; optionally wherein the cancer is breast cancer, malignant brain tumors, colon cancer, small-cell lung cancer, non-small-cell lung cancer, bladder cancer, ovarian cancer, prostate cancer, chronic lymphoid leukemia, lymphoma, myeloma, acute myeloid leukemia, secondary pancreatic cancer or secondary brain metastases.

45-46. (canceled)

47. The method according to claim 45, wherein the breast cancer is HR+/HER2− or HR+/HER2+ advanced or metastatic breast cancer; and the malignant brain tumors are glioblastoma, astrocytoma, or pontine glioma.

48. The method according to claim 44, wherein the patient is administered a pharmaceutical composition comprising the compound; optionally wherein the administration is oral administration.

49. (canceled)

50. The method according to claim 44, further comprising administering an additional therapeutic agent to the patient; optionally wherein the additional therapeutic agent is a PRMT5 inhibitor, a HER2 kinase inhibitor, an aromatase inhibitor, an estrogen receptor antagonist or an alkylating agent; wherein
the aromatase inhibitor is letrozole;
the estrogen receptor antagonist is fulvestrant; and
the alkylating agent is temozolomide.

51-54. (canceled)

55. A method of degrading at least one of a CDK2 protein, a CDK4 protein or a CDK6 protein comprising contacting the CDK2 protein with a compound of claim 1, or a pharmaceutically acceptable salt thereof, or a pharmaceutical composition comprising the compound.

56-57. (canceled)
